(19) United States
(12) Patent Application Publication
(10) Pub. No.: US 2022/0259167 A1
(43) Pub. Date: Aug. 18, 2022

(54) FUROSEMIDE ANALOGUES AND COMPOSITIONS AND USES THEREOF FOR TREATMENT OF ALZHEIMER'S DISEASE

(71) Applicant: UNIVERSITY HEALTH NETWORK, Toronto (CA)

(72) Inventors: Donald Fredric WEAVER, Toronto (CA); Zhiyu WANG, Toronto (CA); Lisa DOYLE, Toronto (CA); Laura VILLAR, Toronto (CA); Prachi S. VILEKAR, Mississauga (CA)

(21) Appl. No.: 17/620,103

(22) PCT Filed: Jun. 26, 2020
(86) PCT No.: PCT/CA2020/050891

§ 371 Date: Dec. 16, 2021

Related U.S. Application Data
(60) Provisional application No. 62/866,735, filed on Jun. 26, 2019.
(60) Provisional application No. 62/985,547, filed on Mar. 5, 2020.

(51) Int. Cl. C07D 307/52; C07D 413/12; C07D 333/22; C07C 229/56; C07D 277/28; C07D 405/12; A61P 25/28

(52) U.S. Cl. CPC C07D 307/52; C07D 413/12; C07D 333/22; A61P 25/28; C07D 277/28; C07D 405/12; C07C 229/56

(57) ABSTRACT

The present application provides furosemide analogues of the general formula Z having activity as anti-Aβ aggregation agents and/or as inhibitors of Aβ induced neuroinflammation.

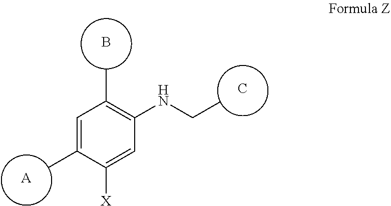

These compounds are useful in preventing, delaying and/or treating Alzheimer's Disease. Accordingly, the present application further provides pharmaceutical compositions and method for preventing, delaying and/or treating Alzheimer's Disease.

### CROSS-REFERENCE TO RELATED APPLICATIONS

The present application claims priority from U.S. Provisional Patent Application No. 62/866,735, filed Jun. 26, 2019, and U.S. Provisional Patent Application No. 62/985,547, filed Mar. 5, 2020, which are incorporated herein in their entirety.

### FIELD OF THE INVENTION

The present application pertains to the field of furosemide analogues. More particularly, the present application relates to furosemide analogues useful in preventing, delaying or treating Alzheimer's Disease.

### INTRODUCTION

Alzheimer's disease (AD) is characterized by the accumulation of the beta-amyloid protein or Aβ, in a fibrillar form, existing as extracellular amyloid plaques and as amyloid within the walls of cerebral blood vessels. Fibrillar Aβ amyloid deposition in AD is believed to be detrimental to the patient and eventually leads to toxicity and neuronal cell death, characteristic hallmarks of AD.

Aβ plaque, caused by protein-misfolding, is a hallmark of AD and, thus, AD can be classified as a proteopathic disease. The formation of Aβ plaque is believed to enhance the level of inflammatory cytokines and over-activate the inflammation response. Therefore, AD can also be classified as an immunopathic disease. FIG. 1 illustrates the proteopathic and immunopathic aspects of AD (Block M. BMC Neuroscience, 2006, 9, S2-S8).

Much work in AD has been accomplished, but little is conventionally known about compounds or agents for therapeutic regimes to arrest amyloid formation, deposition, accumulation and/or persistence that occurs in Alzheimer's disease and other amyloidoses.

Therefore, a need remains for new compounds or agents for therapeutic regimes to arrest or reverse amyloid formation, deposition, accumulation and/or persistence that occurs in Alzheimers disease and other amyloidoses. A need also remains for new compounds or agents for therapeutic regimens to arrest or reverse neuroinflammation that occurs in Alzheimer's disease and other amyloidosis.

The above information is provided for the purpose of making known information believed by the applicant to be of possible relevance to the present invention. No admission is necessarily intended, nor should be construed, that any of the preceding information constitutes prior art against the present invention.

### SUMMARY

An object of the present application is to provide compounds and compositions useful in preventing, delaying or treating Alzheimer's Disease. In accordance with an aspect of the present invention, there is provided a compound, which is a compound of Formula Z:

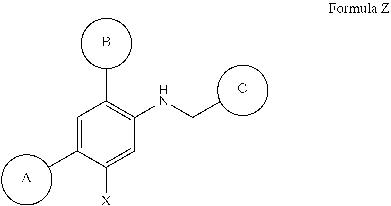

where:

A represents functional region 1, which is optionally present and when present includes both a hydrogen bond donor and a hydrogen bond acceptor;

B represents functional region 2, which comprises a functional group or groups for interaction with positively charged amino acid side chains and, optionally, a hydrogen bond acceptor;

C represents functional region 3, which comprises an aromatic functional group and, optionally, a hydrogen bond acceptor; and

X represents an alkoxy group, such as a methoxy group, or a halide, such as F, Br or Cl,

or a pharmaceutically acceptable salt, solvate or hydrate thereof,
wherein the compound of Formula Z is selected from the group consisting of compounds of Formula I, II, III and IV:

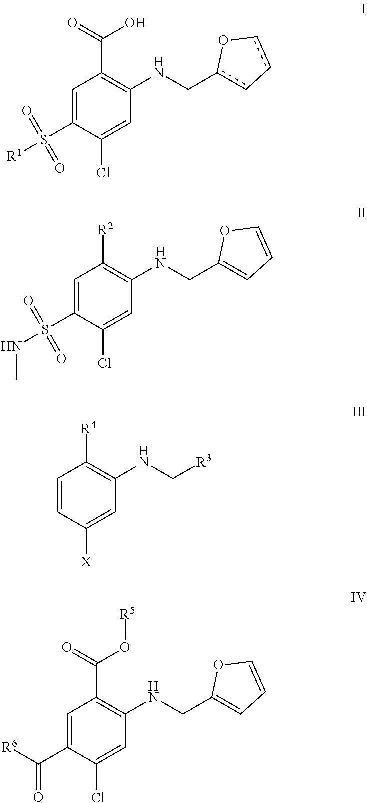

where

### R¹ is

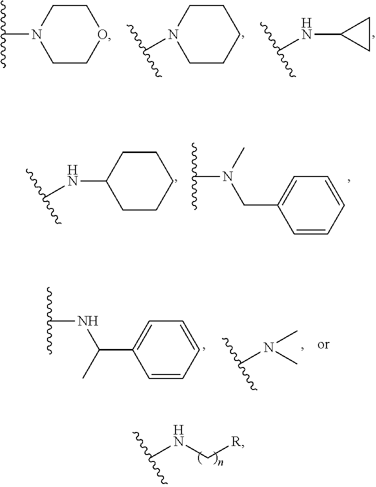

where n is an integer from 0 to 5 and R is CH₃, phenyl or phenyl that is para-substituted with CH₃, OCH₃ or CH(CF₃)OH;

### R² is

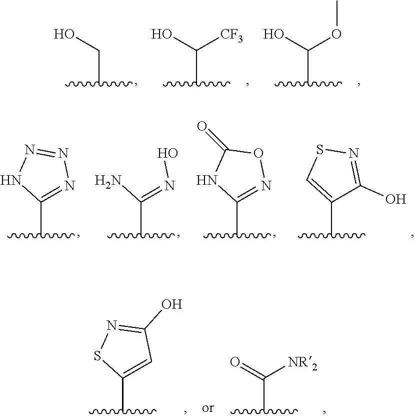

where each R′ is independently H or CH₃;

### R³ is

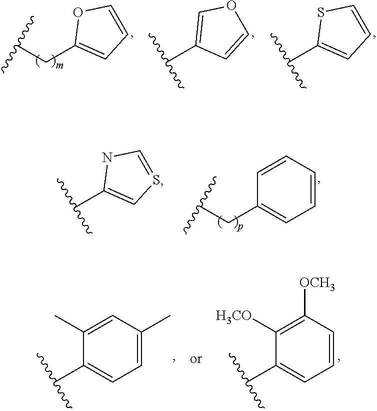

where m is an integer from 0 to 2 and p is an integer of 0 or 1;
R⁴ is —COOH or —CH(CF₃)OH;
R⁵ is H or C₁-C₄ alkyl, preferably H or methyl;
R⁶ is NR⁷R⁸, where each R⁷ and R⁸ is independently selected from H, C₁-C₄ alkyl (preferably methyl or ethyl) or phenyl, or where R⁷ and R⁸ together with the N to which they are bound form a six-membered heterocycle that optionally includes O; and
the dashed lines represent bonds that may be present or absent,
or a pharmaceutically acceptable salt, solvate or hydrate thereof.

In accordance with other embodiments, there is provided a compound of Formula Z, which has the structure of formula I, II, or IIIa:

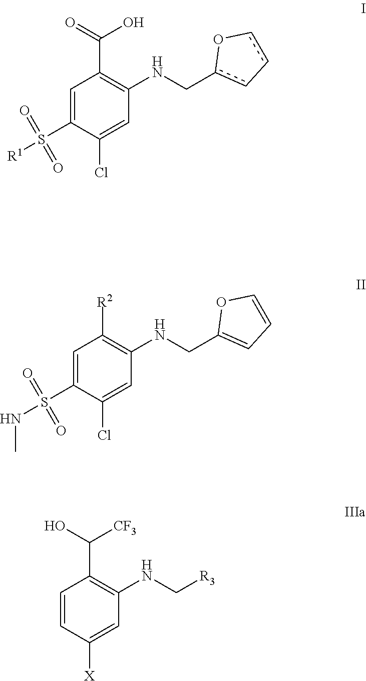

where
R¹ is NHCH₃,

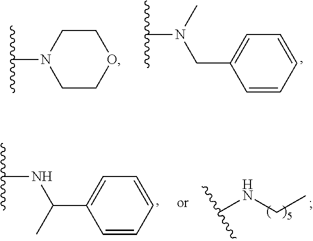

### R² is

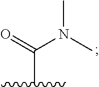 [SMILES: *C(=O)N(C)C]

### R³ is

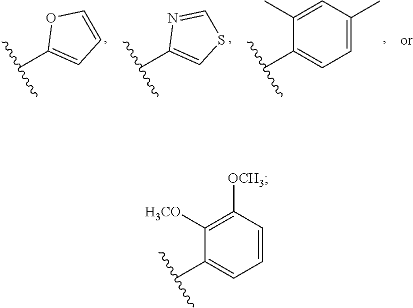

X is alkoxy (e.g., methoxy), F, Cl or Br; and
the dashed lines represent bonds that may be present or absent,
or a pharmaceutically acceptable salt, solvate or hydrate thereof.

In accordance with another aspect of the present application there is provided a use of furosemide, 2-(benzylamino)benzoic acid, the compound of Formula I, II, III or IV, or pharmaceutically acceptable salt, solvate or hydrate thereof, as defined above, for preventing, delaying or treating AD in a subject in need thereof.

In accordance with another aspect of the present application there is provided a method for preventing, delaying or treating AD comprising administering furosemide, 2-(benzylamino)benzoic acid, or the compound of Formula I, II, III or IV, or pharmaceutically acceptable salt, solvate or hydrate thereof, as defined above, to a subject in need thereof.

In accordance with another aspect of the present application there is provided a composition comprising one or more compound of Formula Z:

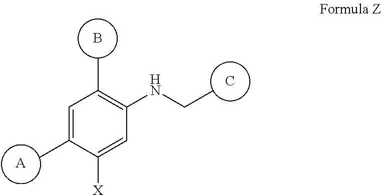

where:

A represents functional region 1, which is optionally present and when present includes both a hydrogen bond donor and a hydrogen bond acceptor;

B represents functional region 2, which comprises a functional group or groups for interaction with positively charged amino acid side chains and, optionally, a hydrogen bond acceptor;

C represents functional region 3, which comprises an aromatic functional group and, optionally, a hydrogen bond acceptor; and

X represents an alkoxy group, such as a methoxy group, or a halide, such as F, Br or Cl,

or a pharmaceutically acceptable salt, solvate or hydrate thereof,
and a pharmaceutically acceptable diluent or excipient,
wherein each of the one or more compounds of Formula Z is selected from the group consisting of compounds of Formula I, II, III and IV:

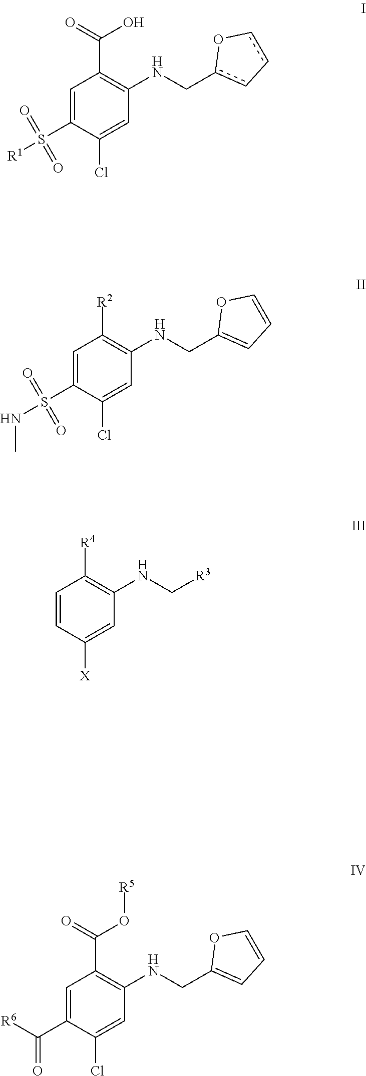

where

### R¹ is

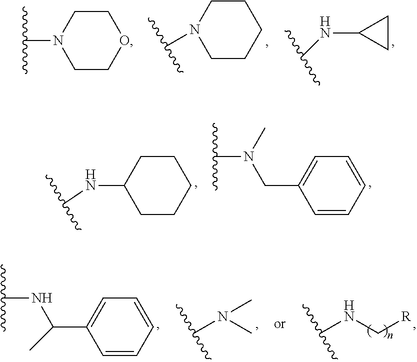

where n is an integer from 0 to 5 and R is CH₃, phenyl or phenyl that is para-substituted with CH₃, OCH₃ or CH(CF₃)OH;

### R² is

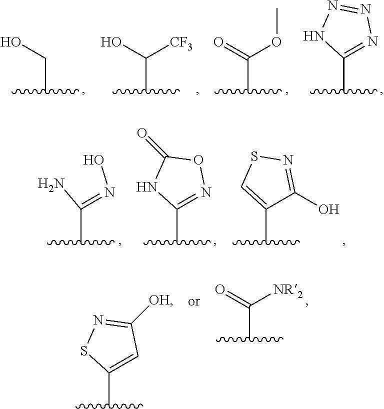

where each R′ is independently H or CH₃;

### R³ is

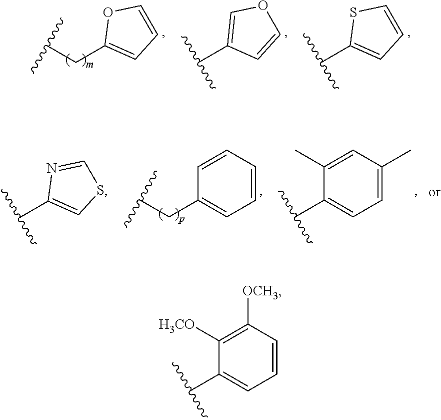

where m is an integer from 0 to 2 and p is an integer of 0 or 1;
R⁴ is —COOH or —CH(CF₃)OH;
R⁵ is H or C₁-C₄ alkyl, preferably H or methyl;
R⁶ is NR⁷R⁸, where each R⁷ and R⁸ is independently selected from H, C₁-C₄ alkyl (preferably methyl or ethyl) or phenyl, or where R⁷ and R⁸ together with the N to which they are bound form a six-membered heterocycle that optionally includes O;

### X is F, Cl or Br; and

the dashed lines represent bonds that may be present or absent.

In accordance with another aspect of the present application there is provided a composition comprising one or more compound of formula I, II, or IIIa:

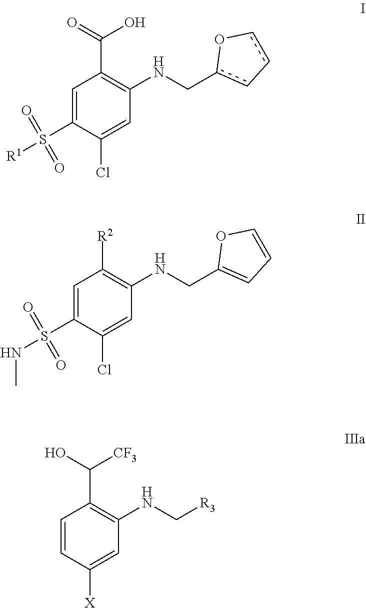

where
R¹ is NHCH₃,

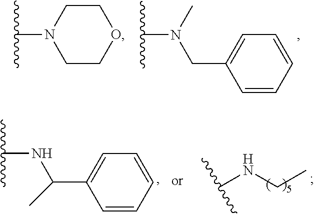

### R² is

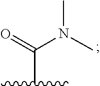 [SMILES: *C(=O)N(C)C]

### R³ is

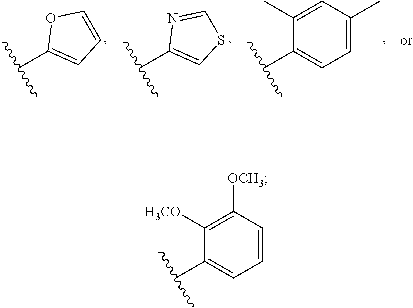

### X is F, Cl or Br; and

the dashed lines represent bonds that may be present or absent,
or a pharmaceutically acceptable salt, solvate or hydrate thereof,
and a pharmaceutically acceptable diluent or excipient.

### BRIEF DESCRIPTION OF THE FIGURES

For a better understanding of the application as described herein, as well as other aspects and further features thereof, reference is made to the following description which is to be used in conjunction with the accompanying drawings.

FIG. 1 schematically depicts proteopathic and immunopathic mechanisms of Alzheimer's Disease.

FIG. 2 schematically depicts an Aβ aggregation assay as employed in Example 2.

FIG. 3 schematically depicts an Aβ fibrillization assay as employed in Example 3.

FIG. 4 schematically depicts the role of microglia involved in neuroinflammation (A) and a neurotoxicity assay (B) as employed in Example 4.

FIG. 5 graphically depicts the results of a neurotoxicity assay of compounds of the present application, showing the SHSY-5y viability 72 h post-addition of supernatant from differentiated THP-1 cells 48 h post-addition of exemplary compounds of the present application.

FIG. 6 graphically depicts the levels of anti-inflammatory cytokines IL-4, IL-10, IL-IRA and Arginase measured as M2 indicators during a neurotoxicity assay of furosemide.

FIG. 7 graphically depicts the IC₅₀ values for anti-Aβ oligomerization activity (μM) for various compounds of the invention having structures of Formula I or Formula II.

FIG. 8 graphically depicts aggregation inhibition percentages for various compounds of the invention having structures of Formula III.

FIG. 9 graphically depicts activity of compounds according to certain embodiments of the invention on TNF-α production from 5 ng/mL LPS stimulated AIM-A9 cells, where the compound concentration was 25 μM.

FIG. 10 graphically depicts activity of compounds according to certain embodiments of the invention on TNF-α production from 5 ng/mL LPS stimulated AIM-A9 cells, where the compound concentration was 8.3 μM.

FIG. 11 graphically depicts activity of compounds according to certain embodiments of the invention on TNF-α production from 5 ng/mL LPS stimulated AIM-A9 cells, where the compound concentration was 2.7 μM.

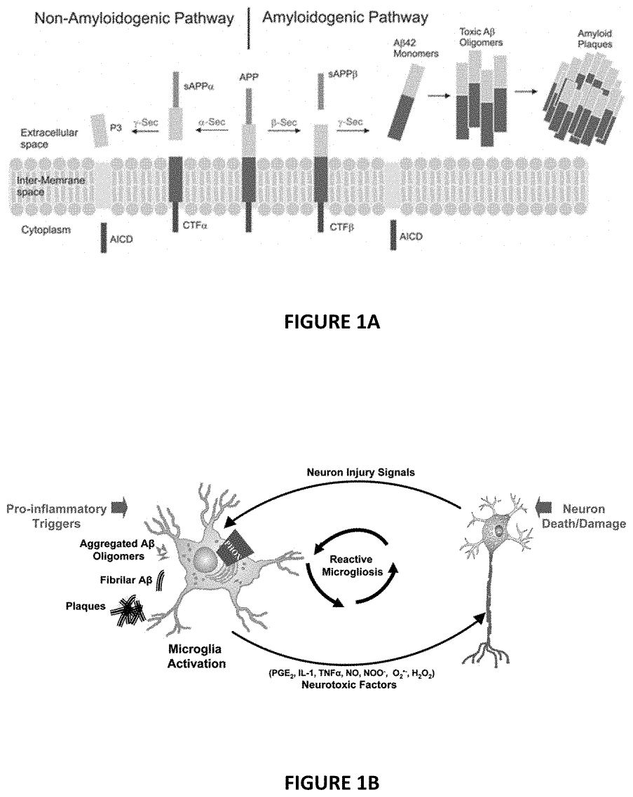

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The term “subject” is used herein interchangeably with the term “patient” to refer to a mammal, such as a human, in need of treatment or potentially in need of treatment.

The terms “treatment” or “treating,” as used herein, mean an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable.

The term “therapeutically effective amount,” “effective amount” or “sufficient amount” of a compound of the present application is a quantity sufficient to, when administered to the subject, including a mammal, for example a human, effect beneficial or desired results, including clinical results, and, as such, an “effective amount” or synonym thereto depends upon the context in which it is being applied.

Moreover, a “treatment” or “prevention” regime of a subject with a therapeutically effective amount of an agent may consist of a single administration, or alternatively comprise a series of applications. For example, the agent may be administered at least once a week. However, in another embodiment, the agent may be administered to the subject from about one time per week to about once daily for a given treatment. The length of the treatment period depends on a variety of factors, such as the severity of the disease, the age of the patient, the concentration and the activity of the agent, or a combination thereof. It will also be appreciated that the effective dosage of the agent used for treatment or prophylaxis may increase or decrease over the course of a particular treatment or prophylaxis regime. Changes in dosage may result and become apparent by standard diagnostic assays known in the art. In some instances, chronic administration may be required.

The term “administering” is defined as any conventional route for administering an agent(s) to a subject for use as is known to one skilled in the art. This may include, for example, administration via the parenteral (i.e., subcutaneous, intradermal, intramuscular, etc.) or mucosal surface route. In other embodiments this may include oral administration. The dose of the agent(s) may vary according to factors such as the health, age, weight and sex of the animal. The dosage regime may be adjusted to provide the optimum dose. One skilled in the art will appreciate that the dosage regime can be determined and/or optimized without undue experimentation.

To “inhibit” or “suppress” or “lower” or “reduce” or “down regulate” a function or activity, is to reduce the function or activity when compared to otherwise same conditions except for a condition or parameter of interest, or alternatively, as compared to another condition or control.

The present application provides compounds and compositions for use in preventing, delaying or treating Alzheimer's disease. A tryptophan metabolite, 3-hydroxylanthranilic acid (3-HAA), is an endogenous molecule with anti-fibrilization activity against Aβ, and which can reduce inflammation progression. 3-HAA was used to identify other compounds having 3-HAA as a backbone that would be candidates for AD treatment and/or prevention. Furosemide was identified as 3-HAA analogue, that can be used to target Aβ misfolding and neuroinflammation.

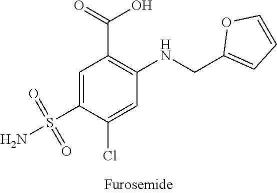 [SMILES: NS(=O)(=O)c1cc(C(=O)O)c(NCc2ccco2)cc1Cl]

A series of furosemide analogues have now been synthesized and demonstrated to also have activity in targeting Aβ misfolding and/or neuroinflammation. The furosemide analogues of the present application have the general structure of Formula Z:

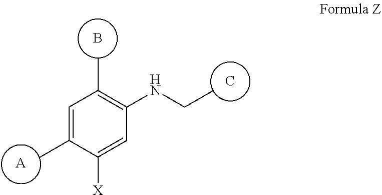

where:

A represents functional region 1, which is optionally present and when present includes both a hydrogen bond donor and a hydrogen bond acceptor;

B represents functional region 2, which comprises a functional group or groups for interaction with positively charged amino acid side chains and, optionally, a hydrogen bond acceptor;

C represents functional region 3, which comprises an aromatic functional group and, optionally, a hydrogen bond acceptor; and

X represents a halide, such as F, Br or Cl.

In functional region 3, the aromatic functional group or groups optionally interact with the target through π-π stacking or cation-π interactions.

Accordingly, the present application provides compounds of Formula Z that are selected from the group consisting of compounds of Formula I, II, III and IV, which are useful in preventing, delaying or treating AD by inhibiting Aβ aggregation and/or reducing or inhibiting Aβ-induced neuroinflammation:

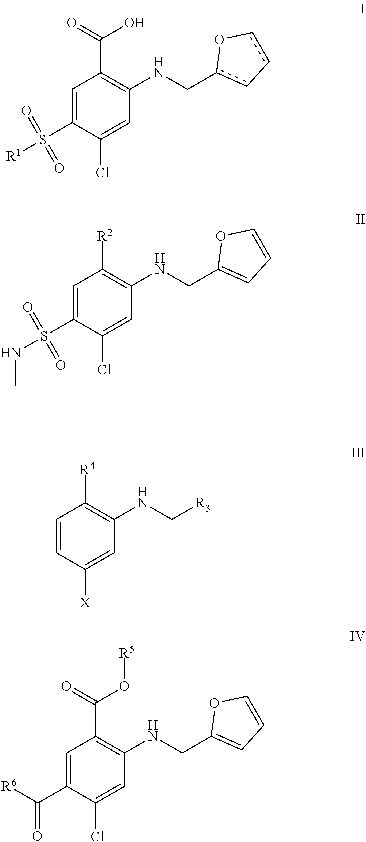

where

### R¹ is

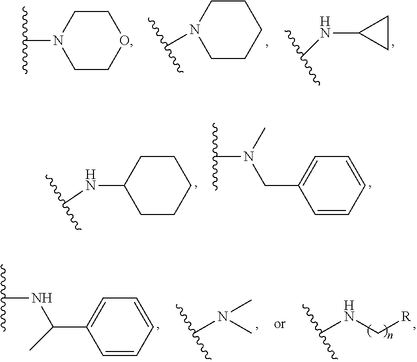

where n is an integer from 0 to 5 and R is CH₃, phenyl or phenyl that is para-substituted with CH₃, OCH₃ or CH(CF₃)OH;

### R² is

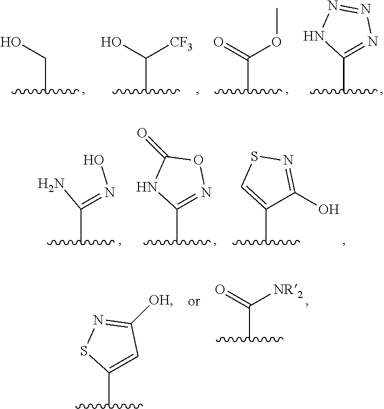

where each R′ is independently H or CH₃;

### R³ is

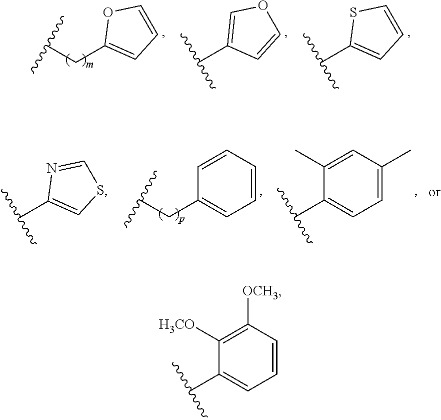

where m is an integer from 0 to 2 and p is an integer of 0 or 1;
R⁴ is —COOH or —CH(CF₃)OH;
R⁵ is H or C₁-C₄ alkyl, preferably H or methyl;
R⁶ is NR⁷R⁸, where each R⁷ and R⁸ is independently selected from H, C₁-C₄ alkyl (preferably methyl or ethyl) or phenyl, or where R⁷ and R⁸ together with the N to which they are bound form a six-membered heterocycle that optionally includes O;

### X is F, Cl or Br; and

the dashed lines represent bonds that may be present or absent.

In certain embodiments, the compound of Formula III is a compound of Formula IIIa:

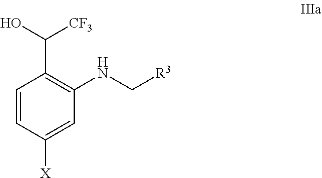

where R³ and X are as defined above.

In certain embodiments, the compound of Formula III is a compound of Formula IIIb:

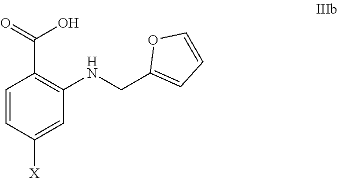

where X is as defined above.

Furosemide and its analogues, including the compounds of Formulae I, II, III and IV and 2-(benzylamino)benzoic acid, have now been found to act as: (i) anti-Aβ aggregation agents, by inhibiting Aβ oligomerization and/or fibrillization; and/or (ii) anti-neuroinflammation agents by rescuing neuronal cells under Aβ induced neuroinflammation.

In a specific example, a compound of formula II showed potent activity, 6.5 μM towards anti-Aβ oligomerization activity. In another specific example, 2-(benzylamino)benzoic acid and a compound of formula IIIa, in which X is Br and R³ is

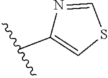 [SMILES: *c1cscn1]

demonstrated anti-neuroinflammation activity by being able to rescue neuronal cells under Aβ-induced neurotoxicity.

Compositions and Use

The present application further provides compositions comprising furosemide, 2-(benzylamino)benzoic acid, a compound of formula Z (such as, a compound of formula I, a compound of formula II, a compound of formula III or a compound of formula IV), or any combination thereof.

The compositions containing these compound(s) can be prepared by known methods for the preparation of pharmaceutically acceptable compositions which can be administered to subjects, such that an effective quantity of the active agent(s) is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the compound(s) in association with one or more pharmaceutically acceptable vehicles or diluents, and/or contained in buffered solutions with a suitable pH and iso-osmotic with the physiological fluids.

The compositions can be formulated for administration to a subject alone or in combination with pharmaceutically acceptable carriers, as noted above, and/or with other pharmaceutically active agents for preventing, delaying or treating AD, the proportion of which is determined by the solubility and chemical nature of the agents, chosen route of administration and standard pharmaceutical practice.

The dosage of the compound(s) and/or compositions can vary depending on many factors such as the pharmacodynamic properties of the agent, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compound(s) can be administered initially in a suitable dosage that may be adjusted as required, depending on the clinical response.

The present application further provides methods of using furosemide, 2-(benzylamino)benzoic acid, a compound of formula Z (such as, a compound of formula I, a compound of formula II, a compound of formula III or a compound of formula IV), or any combination thereof, for preventing, delaying or treating AD. In accordance with certain embodiments, there is provided a method of preventing, delaying or treating AD in a subject in need thereof, comprising administering to the subject a therapeutically effective amount of furosemide, 2-(benzylamino)benzoic acid, a compound of formula I, a compound of formula II, a compound of formula III, or any combination thereof.

Synthesis of Furosemide Analogues

The present application further provides methods for synthesizing compounds of Formula Z. Details of exemplary, non-limiting, methods for synthesizing these compounds are found in the following examples.

In certain embodiments there is provided a method of synthesizing compounds of Formula Z, in which the functional group at region A is varied. These compounds have the structure of Formula I, as depicted above. An example of this synthetic method is provided in Scheme I:

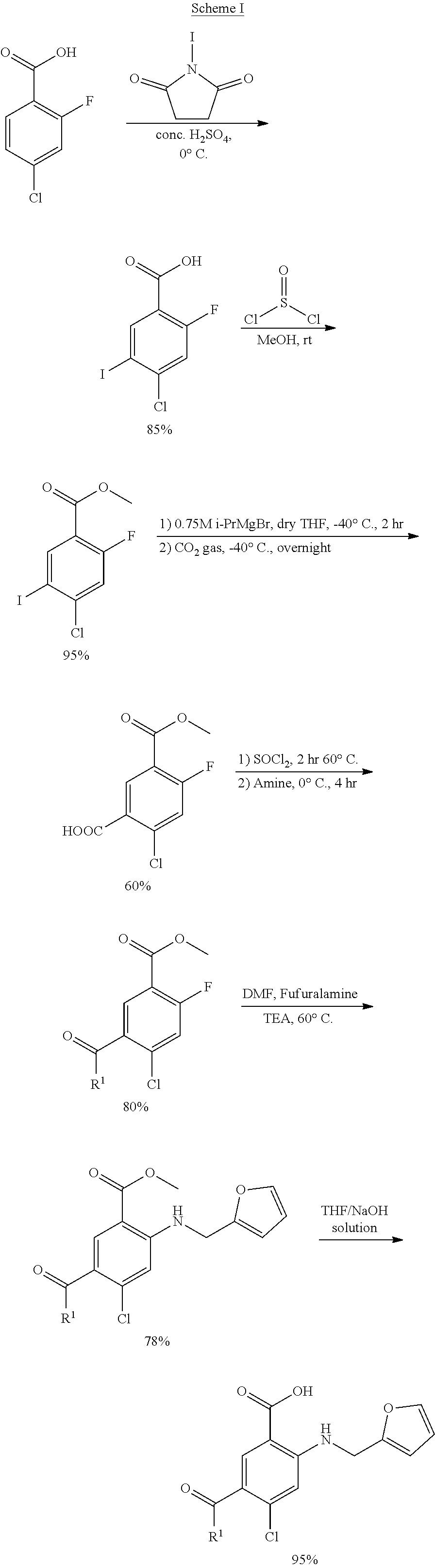

In other embodiments there is provided a method of synthesizing compounds of Formula Z, in which the functional group at region B is varied. These compounds have the structure of Formula II, as depicted above. An example of this synthetic method is provided in Scheme II:

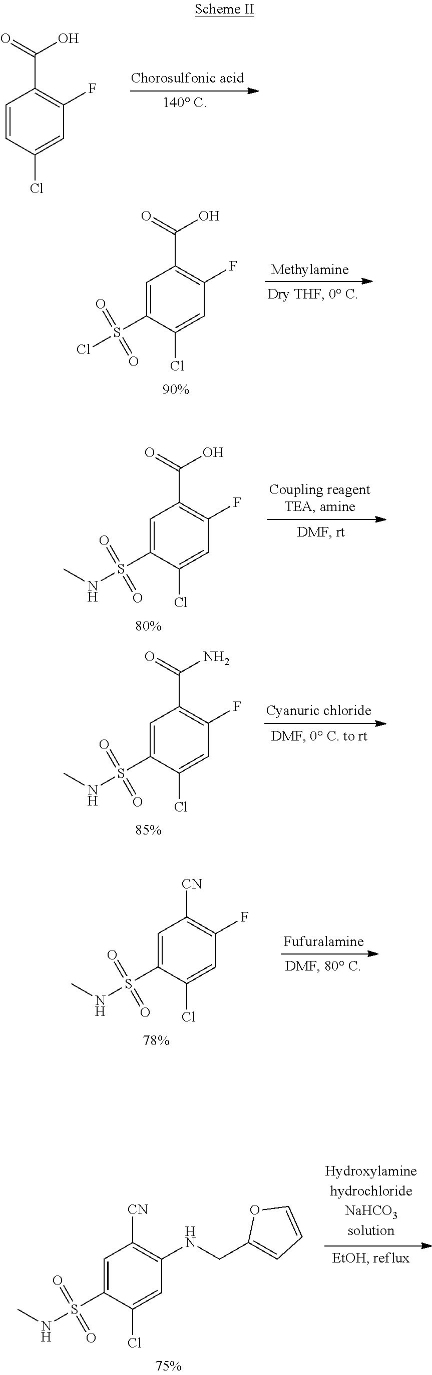 [SMILES: O=C(O)c1ccc(Cl)cc1F ; O=C(O)c1cc(S(=O)(=O)Cl)c(Cl)cc1F ; CNS(=O)(=O)c1cc(C(=O)O)c(F)cc1Cl ; CNS(=O)(=O)c1cc(C(N)=O)c(F)cc1Cl ; CNS(=O)(=O)c1cc(C#N)c(F)cc1Cl ; CNS(=O)(=O)c1cc(C#N)c(NCc2ccco2)cc1Cl]

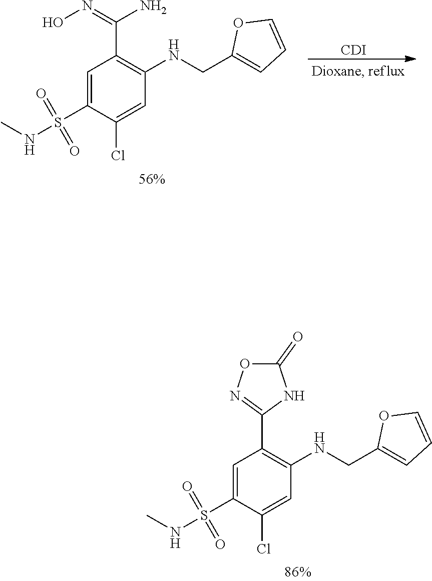

Specific examples of synthetic methods for making the furosemide analogues of the present application are provided in the Examples section below.

To gain a better understanding of the invention described herein, the following examples are set forth. It should be understood that these examples are for illustrative purposes only. Therefore, they should not limit the scope of this invention in any way.

### EXAMPLES

### Example 1: Synthesis and Characterization of Anti-Aβ Aggregation and/or Anti-Neuroinflammation Compounds

The following synthetic schemes include reference numerals to compounds. The descriptions of the syntheses that follow include these reference numerals as well as the compound identifier numbers used in the activity studies that follow.

Furthermore, throughout the specification and figures, certain synonymous nomenclature is used to refer to compound designators that have alphanumeric prefixes. A compound denoted “WW016” may also be denoted “W016”, and it may be further denoted with inclusion of an optional series designator “S” followed by Roman numerals, e.g., “SIIIa-W016” or “SIIIa-WW016”. These designations are all synonymous and chemical structure associated with each set of synonymous compound designators will be clear from one or more of the following: the presentation of the corresponding structure or Series/R-group key above the associated bar in a relevant bar graph Figure; the presentation of the corresponding structure or Series/R-group key in the associated row in a Table; or reference to “WW ###” nomenclature alongside the numerically consecutive synthetic nomenclature for the associated synthesis in this Example 1.

Synthesis and Characterisation of Compounds of Formula I

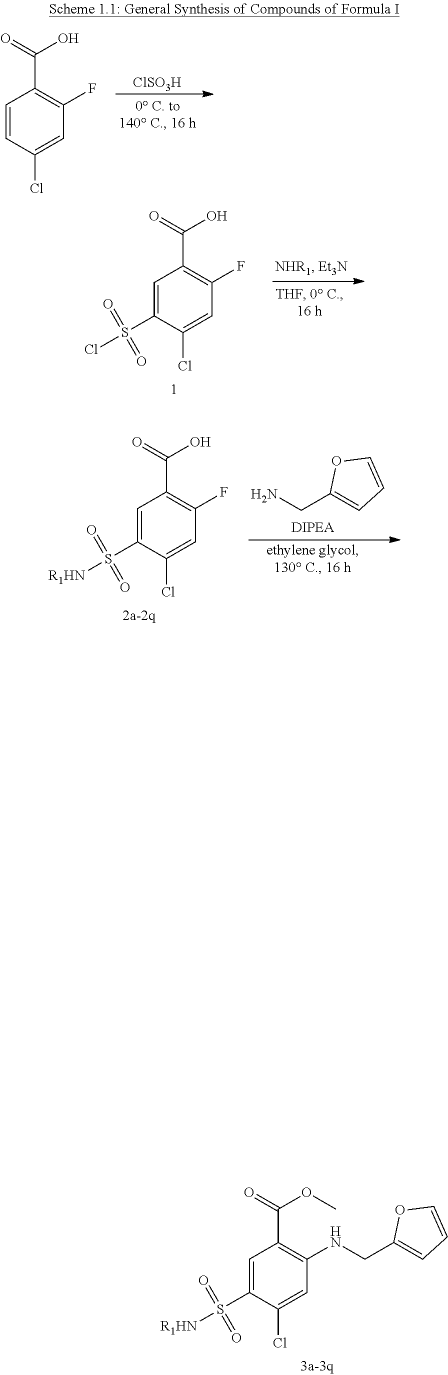

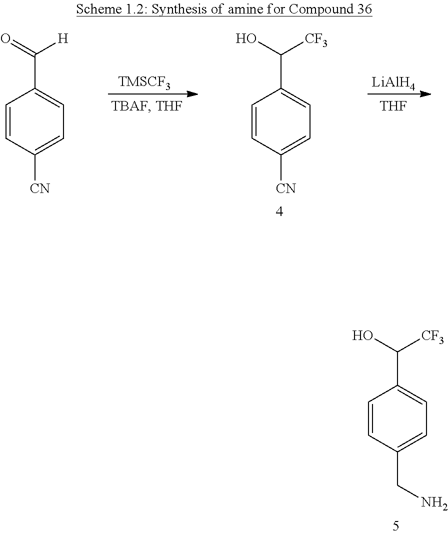 [SMILES: "TMSCF3" ; N#Cc1ccc(C=O)cc1 ; N#Cc1ccc(C(O)C(F)(F)F)cc1 ; "LiAlH4" ; NCc1ccc(C(O)C(F)(F)F)cc1 ; "THF"]

General Procedure A for Preparation of Compounds 2a-2c.

4-Chloro-5-(chlorosulfonyl)-2-fluorobenzoic acid (1 eq.) was dissolved in anhydrous THF (0.3 M) and cooled to 0° C. Triethylamine (3 eq.) was added to the reaction mixture. The appropriate amine (1.2 eq.) was dissolved by THF and added to reaction slowly. The reaction was allowed to warm to room temperature and monitored by TLC. Upon completion of the reaction, the mixture was diluted with EtOAc/H₂O and layers separated. The pH of the aqueous phase was adjusted to 3 and extracted with EtOAc. The organic layers were washed with brine, dried over Na₂SO₄ and concentrated under reduced pressure. Column chromatography gave the desired compounds 2a-2q (20-88%).

General Procedure B for Preparation of Compounds 3a-3c.

Intermediates 2a-2q (1 eq.) were dissolved in ethylene glycol (0.3 M) and to this, furfurylamine (3 eq.) and DIPEA (2 eq.) were added. The reaction mixture was heated to 130° C. for 16 h. The mixture was diluted with EtOAc/H₂O and layers were separated. The pH of the aqueous phase was adjusted to 3 and extracted with EtOAc. The organic layers were washed with brine, dried over Na₂SO₄ and concentrated under reduced pressure. Column chromatography gave the compounds 3a-3q (33-82%).

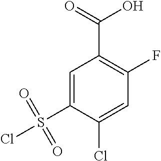 [SMILES: O=C(O)c1cc(S(=O)(=O)Cl)c(Cl)cc1F]

### 4-Chloro-5-(chlorosulfonyl)-2-fluorobenzoic Acid (Intermediate 1)

Chlorosulfonic acid (4.09 g, 35.09 mmol, 6 eq.) was cooled to 0° C. To this, 4-chloro-2-fluorobenzoic acid (1.02 g, 5.85 mmol, 1 eq.) was added, portion-wise. The reaction was then heated to 140° C. and stirred for 5 h. The reaction was monitored by TLC. After TLC indicated consumption of starting material, the reaction was cooled to −10° C. and ice was added to the mixture. A white solid precipitated, was washed with water and placed in desiccator to dry. The title product was obtained as a yellow solid (1.44 g, 90%). R_{f} 0.3, hexane-EtOAc-AcOH 9:1:0.05.

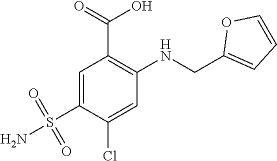 [SMILES: NS(=O)(=O)c1cc(C(=O)O)c(NCc2ccco2)cc1Cl]

### 4-Chloro-2-((furan-2-ylmethyl)amino)-5-sulfamoylbenzoic Acid (Furosemide)

According to the General Procedures A and B, the title compound (126 mg, 76% over 2 steps) was obtained as a white solid.

¹H NMR (300 MHz, DMSO-d₆) δ (ppm) 13.36 (s, 1H), 8.62 (t, J=5.9 Hz, 1H), 8.38 (s, 1H), 7.62 (d, J=1.8 Hz, 1H), 7.34 (s, 2H), 7.06 (s, 1H), 6.46-6.26 (m, 2H), 4.58 (d, J=5.8 Hz, 2H); HRMS (ESI, m/z) Calculated for C₁₂H₁₀ClN₂O₅S [M−H]⁻: 328.9999, found: 328.9998.

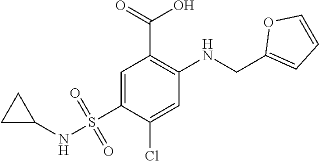 [SMILES: O=C(O)c1cc(S(=O)(=O)NC2CC2)c(Cl)cc1NCc1ccco1]

### 4-Chloro-5-(N-cyclopropylsulfamoyl)-2-((furan-2-ylmethyl)amino)benzoic Acid (3b, WW030)

According to the General Procedures A and B, the title compound (270 mg, 59% over 2 steps) was obtained as a white solid.

¹H NMR (300 MHz, DMSO-d₆) δ (ppm) 13.46 (s, 1H), 8.72 (t, J=5.4 Hz, 1H), 8.40 (s, 1H), 7.92 (d, J=2.3 Hz, 1H), 7.63 (m, 1H), 7.08 (s, 1H), 6.51-6.28 (m, 2H), 4.59 (d, J=5.7 Hz, 2H), 2.13 (m, 1H), 0.54-0.18 (m, 4H); HRMS (ESI, m/z) Calculated for C₁₅H₁₄ClN₂O₅S [M−H]⁻: 369.0312, found: 369.0312.

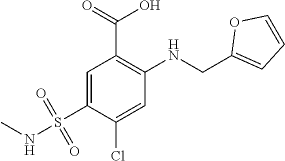 [SMILES: CNS(=O)(=O)c1cc(C(=O)O)c(NCc2ccco2)cc1Cl]

### 4-chloro-2-((furan-2-ylmethyl) amino)-5-(N-methylsulfamoyl) Benzoic Acid (3c, WW032)

According to the General Procedures A and B, the title compound (126 mg, 57% over 2 steps) was obtained as a white solid.

¹H NMR (300 MHz, DMSO-d₆) δ (ppm) 13.44 (s, 1H), 8.69 (t, J=5.9 Hz, 1H), 8.34 (s, 1H), 7.63 (d, J=1.8 Hz, 1H), 7.37 (q, J=4.9 Hz, 1H), 7.08 (s, 1H), 6.46-6.34 (m, 2H), 4.59 (d, J=5.8 Hz, 2H), 2.40 (d, J=4.9 Hz, 3H); HRMS (ESI, m/z) Calculated for C₁₃H₁₂ClN₂O₅S [M−H]⁻: 343.0155, found: 343.0159

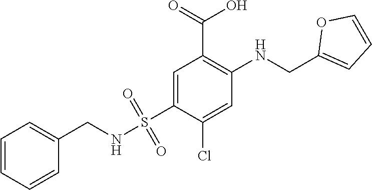 [SMILES: O=C(O)c1cc(S(=O)(=O)NCc2ccccc2)c(Cl)cc1NCc1ccco1]

### 5-(N-benzylsulfamoyl)-4-chloro-2-((furan-2-ylmethyl)amino)benzoic Acid (3d, WW035)

According to the General Procedures A and B, the title compound (390 mg, 57% over 2 steps) was obtained as a white solid.

¹H NMR (300 MHz, DMSO-d₆) δ (ppm) 8.81-8.60 (m, 1H), 8.29 (s, 1H), 8.12 (t, J=6.3 Hz, 1H), 7.64 (dd, J₁=1.9 Hz, J₂=0.9 Hz, 1H), 7.31-7.10 (m, 5H), 6.98 (s, 1H), 6.44 (dd, J₁=3.2 Hz, J₂=1.9 Hz, 1H), 6.40-6.30 (m, 1H), 4.56 (d, J=5.8 Hz, 2H), 4.01 (d, J=6.3 Hz, 2H); HRMS (ESI, m/z) Calculated for C₁₉H₁₆ClN₂O₅S [M−H]⁻: 419.0468, found: 419.0469.

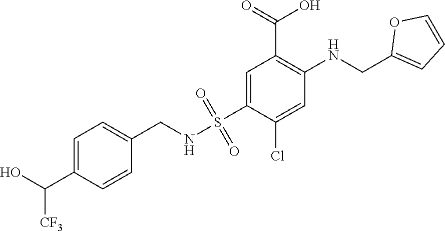 [SMILES: O=C(O)c1cc(S(=O)(=O)NCc2ccc(C(O)C(F)(F)F)cc2)c(Cl)cc1NCc1ccco1]

### 4-Chloro-2-((furan-2-ylmethyl)amino)-5-(N-(4-(2,2,2-trifluoro-1-hydroxyethyl)benzyl) sulfamoyl)benzoic Acid (3e, WW036)

According to the General Procedures A and B, the title compound (362 mg, 31% over 2 steps) was obtained as a white solid.

¹H NMR (300 MHz, DMSO-d₆) δ (ppm) 13.39 (s, 1H), 8.68 (t, J=5.8 Hz, 1H), 8.34 (s, 1H), 8.16 (t, J=6.2 Hz, 1H), 7.64 (s, 1H), 7.36 (d, J=8.0 Hz, 2H), 7.26 (d, J=8.2 Hz, 2H), 7.03 (s, 1H), 6.79 (d, J=5.6 Hz, 1H), 6.48-6.31 (m, 2H), 5.08 (p, J=7.3 Hz, 1H), 4.57 (d, J=5.7 Hz, 2H), 4.02 (d, J=6.1 Hz, 2H); HRMS (ESI, m/z) Calculated for C₂₁H₁₇ClF₃N₂O₆S [M−H]⁻: 517.0448, found: 517.0451.

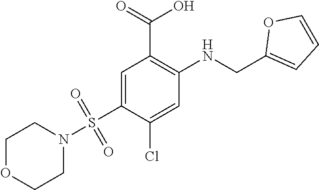 [SMILES: O=C(O)c1cc(S(=O)(=O)N2CCOCC2)c(Cl)cc1NCc1ccco1]

### 4-Chloro-2((furan-2-ylmethyl) amino)-5-(morpholinosulfony)benzoic Acid (3f, WW049)

According to the General Procedures A and B, the title compound (123 mg, 50% over 2 steps) was obtained as a white solid.

¹H NMR (300 MHz, DMSO-d₆) δ (ppm) 13.53 (s, 1H), 8.78 (t, J=5.8 Hz, 1H), 8.33 (s, 1H), 7.67-7.57 (m, 1H), 7.13 (s, 1H), 6.49-6.33 (m, 2H), 4.60 (d, J=5.8 Hz, 2H), 3.67-3.49 (m, 4H), 3.13-2.97 (m, 4H); HRMS (ESI, m/z) Calculated for C₁₆H₁₆ClN₂O₆S [M−H]⁻: 399.0417, found: 399.0420.

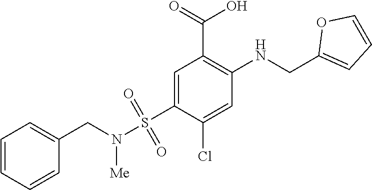 [SMILES: CN(Cc1ccccc1)S(=O)(=O)c1cc(C(=O)O)c(NCc2ccco2)cc1Cl]

### 5-(N-Benzyl-N-methylsulfamoyl)-4-chloro-2-((furan-2-ylmethyl) amino) benzoic Acid (3g, WW052)

According to the General Procedures A and B, the title compound (269 mg, 34% over 2 steps) was obtained as a white solid.

¹H NMR (300 MHz, DMSO-d₆) δ (ppm) 8.82 (d, J=5.1 Hz, 1H), 8.41 (s, 1H), 7.64 (s, 1H), 7.40-7.24 (m, 5H), 7.14 (s, 1H), 6.48-6.36 (m, 2H), 4.61 (d, J=5.8 Hz, 2H), 4.32 (s, 2H), 2.61 (s, 3H); HRMS (ESI, m/z) Calculated for C₂₀H₁₈ClN₂O₅S [M−H]⁻: 433.0625, found: 433.0625.

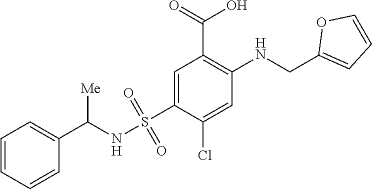 [SMILES: CC(NS(=O)(=O)c1cc(C(=O)O)c(NCc2ccco2)cc1Cl)c1ccccc1]

### 4-Chloro-2-((furan-2-ylmethyl) amino)-5-(N-(1-phenylethyl) sulfamoyl) benzoic Acid (3 h, WW053)

According to the General Procedure A and B, the title compound (188 mg, 24% over 2 steps) was obtained as a white solid.

¹H NMR (300 MHz, DMSO-d₆) δ (ppm) 13.30 (s, 1H), 8.60 (t, J=5.7 Hz, 1H), 8.20 (s, 1H), 8.13 (d, J=8.5 Hz, 1H), 7.65 (d, J=1.0 Hz, 1H), 7.20-7.01 (m, 5H), 6.88 (s, 1H), 6.49-6.29 (m, 2H), 4.53 (d, J=5.8 Hz, 2H), 4.28 (dt, J₁=14.3 Hz, J₂=7.0 Hz, 1H), 1.29 (d, J=7.0 Hz, 3H); HRMS (ESI, m/z) Calculated for C₂₀H₁₈ClN₂O₅S [M−H]⁻: 433.0625, found: 433.0624.

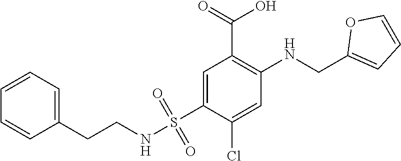 [SMILES: O=C(O)c1cc(S(=O)(=O)NCCc2ccccc2)c(Cl)cc1NCc1ccco1]

### 4-Chloro-2-((furan-2-ylmethyl) amino)-5-(N-phenethylsulfamoyl) Benzoic Acid (3i, WW056)

According to the General Procedure A and B, the title compound (612 mg, 77% over 2 steps) was obtained as a white solid.

¹H NMR (300 MHz, DMSO-d₆) δ (ppm) 13.41 (s, 1H), 8.68 (t, J=6.0 Hz, 1H), 8.33 (s, 1H), 7.73-7.52 (m, 2H), 7.26-6.96 (m, 6H), 6.49-6.27 (m, 2H), 4.58 (d, J=5.9 Hz, 2H), 3.00 (q, J=7.2 Hz, 2H), 2.68 (q, J=7.7 Hz, 2H); HRMS (ESI, m/z) Calculated for C₂₀H₁₈ClN₂O₅S [M−H]⁻: 433.0625, found: 433.0625.

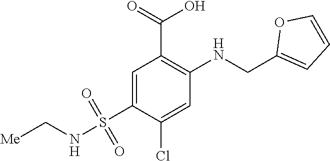 [SMILES: CCNS(=O)(=O)c1cc(C(=O)O)c(NCc2ccco2)cc1Cl]

### 4-Chloro-5-(N-ethylsulfamoyl)-2-((furan-2-ylmethyl) amino) Benzoic Acid (3j, WW057)

According to the General Procedures A and B, the title compound (127 mg, 19% over 2 steps) was obtained as a white solid.

¹H NMR (300 MHz, DMSO-d₆) δ (ppm) 13.44 (s, 1H), 8.70 (t, J=5.7 Hz, 1H), 8.35 (s, 1H), 7.63 (dd, J₁=1.9 Hz, J₂=0.9 Hz, 1H), 7.52 (t, J=5.7 Hz, 1H), 7.07 (s, 1H), 6.46-6.32 (m, 2H), 4.58 (d, J=5.7 Hz, 2H), 2.80 (p, J=7.2 Hz, 2H), 0.96 (t, J=7.2 Hz, 3H); HRMS (ESI, m/z) Calculated for C₁₄H₁₄ClN₂O₅S [M−H]⁻: 357.0312, found: 357.0314.

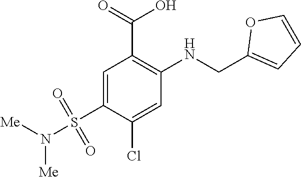 [SMILES: CN(C)S(=O)(=O)c1cc(C(=O)O)c(NCc2ccco2)cc1Cl]

### 4-Chloro-5-(N, N-dimethylsulfamoyl)-2-((furan-2-ylmethyl) amino) Benzoic Acid (3k, WW062)

According to the General Procedures A and B, the title compound (238 mg, 36% over 2 steps) was obtained as a white solid.

¹H NMR (300 MHz, DMSO-d₆) δ (ppm) 13.50 (s, 1H), 8.74 (t, J=5.9 Hz, 1H), 8.33 (s, 1H), 7.63 (dd, J₁=1.8 Hz, J₂=0.9 Hz, 1H), 7.11 (s, 1H), 6.47-6.35 (m, 2H), 4.60 (d, J=5.8 Hz, 2H), 2.72 (s, 6H); HRMS (ESI, m/z) Calculated for C₁₄H₁₄ClN₂O₅S [M−H]⁻: 357.0312, found: 357.0311.

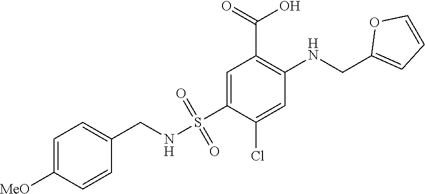 [SMILES: COc1ccc(CNS(=O)(=O)c2cc(C(=O)O)c(NCc3ccco3)cc2Cl)cc1]

### 4-Chloro-2-((furan-2-ylmethyl) amino)-5-(N-(4-methoxybenzyl) sulfamoyl) Benzoic Acid (31, WW063)

According to the General Procedures A and B, the title compound (167 mg, 21% over 2 steps) was obtained as a white solid.

¹H NMR (300 MHz, DMSO-d₆) δ (ppm) 13.35 (s, 1H), 8.64 (t, J=5.9 Hz, 1H), 8.23 (s, 1H), 8.02 (t, J=6.2 Hz, 1H), 7.63 (s, 1H), 7.08 (d, J=8.4 Hz, 2H), 6.97 (s, 1H), 6.73 (d, J=8.4 Hz, 2H), 6.46-6.32 (m, 2H), 4.56 (d, J=5.8 Hz, 2H), 3.94 (d, J=6.2 Hz, 2H), 3.67 (s, 3H); HRMS (ESI, m/z) Calculated for C₂₀H₁₈ClN₂O₆S [M−H]⁻: 449.0574, found: 449.0575.

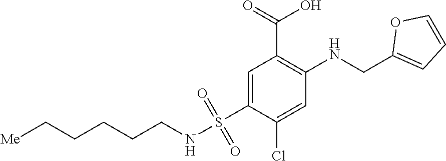 [SMILES: CCCCCCNS(=O)(=O)c1cc(C(=O)O)c(NCc2ccco2)cc1Cl]

### 4-Chloro-2-((furan-2-ylmethyl) amino)-5-(N-hexylsulfamoyl) Benzoic Acid (3m, WW064)

According to the General Procedures A and B, the title compound (561 mg, 74% over 2 steps) was obtained as a white solid.

¹H NMR (300 MHz, DMSO-d₆) δ (ppm) 13.42 (s, 1H), 8.71 (s, 1H), 8.34 (s, 1H), 7.73-7.43 (m, 2H), 7.06 (s, 1H), 6.39 (dd, J₁=14.0 Hz, J₂=2.3 Hz, 2H), 4.57 (d, J=5.5 Hz, 2H), 2.74 (q, J=6.5 Hz, 2H), 1.40-1.26 (m, 2H), 1.19-1.03 (m, 6H), 0.79 (t, J=6.6 Hz, 3H); HRMS (ESI, m/z) Calculated for C₁₈H₂₂ClN₂O₅S [M−H]⁻: 413.0938, found: 413.0939.

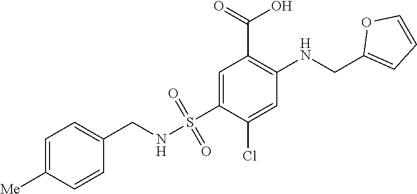 [SMILES: Cc1ccc(CNS(=O)(=O)c2cc(C(=O)O)c(NCc3ccco3)cc2Cl)cc1]

### 4-Chloro-2-((furan-2-ylmethyl) amino)-5-(N-(4-methylbenzyl) sulfamoyl) Benzoic Acid (3n, WW065)

According to the General Procedure A and B, the title compound (259 mg, 33% over 2 steps) was obtained as a white solid.

¹H NMR (300 MHz, DMSO-d₆) δ (ppm) 9.84 (s, 1H), 8.32 (s, 1H), 7.87 (t, J=6.0 Hz, 1H), 7.62 (s, 1H), 7.14-6.97 (m, 4H), 6.82 (s, 1H), 6.46-6.27 (m, 2H), 4.46 (d, J=4.9 Hz, 2H), 3.92 (d, J=6.1 Hz, 2H), 2.22 (s, 3H); HRMS (ESI, m/z) Calculated for C₂₀H₁₈ClN₂O₅S [M−H]⁻: 433.0625, found: 433.0623.

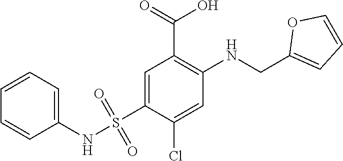 [SMILES: O=C(O)c1cc(S(=O)(=O)Nc2ccccc2)c(Cl)cc1NCc1ccco1]

### 4-Chloro-2-((furan-2-ylmethyl) amino)-5-(N-phenylsulfamoyl) benzoic acid (3o, Compound 80)

According to the General Procedures A and B, the title compound (600 mg, 81% over 2 steps) was obtained as a white solid.

¹H NMR (300 MHz, DMSO-d₆) δ (ppm) 13.49 (s, 1H), 10.35 (s, 1H), 8.70 (t, J=5.8 Hz, 1H), 8.40 (s, 1H), 7.60 (dd, J₁=1.9 Hz, J₂=0.9 Hz, 1H), 7.26-7.17 (m, 2H), 7.10-6.92 (m, 4H), 6.43-6.30 (m, 2H), 4.54 (d, J=5.7 Hz, 2H); HRMS (ESI, m/z) Calculated for C₁₈H₁₄ClN₂O₅S [M−H]⁻: 405.0312, found: 405.0312.

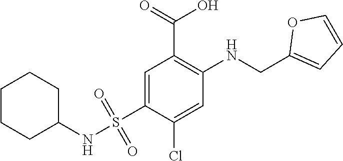 [SMILES: O=C(O)c1cc(S(=O)(=O)NC2CCCCC2)c(Cl)cc1NCc1ccco1]

### 4-Chloro-5-(N-cyclohexylsulfamoyl)-2-((furan-2-ylmethyl) amino) Benzoic Acid (3p, WW082)

According to the General Procedures A and B, the title compound (409 mg, 54% over 2 steps) was obtained as a white solid.

¹H NMR (300 MHz, DMSO-d₆) δ (ppm) 13.45 (s, 1H), 8.70 (s, 1H), 8.37 (s, 1H), 7.67-7.50 (m, 2H), 7.06 (s, 1H), 6.48-6.32 (m, 2H), 4.57 (d, J=5.5 Hz, 2H), 1.73-0.81 (m, 11H); HRMS (ESI, m/z) Calculated for C₁₈H₂₀ClN₂O₅S [M−H]⁻: 411.0783, found: 411.0783.

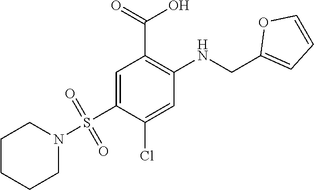 [SMILES: O=C(O)c1cc(S(=O)(=O)N2CCCCC2)c(Cl)cc1NCc1ccco1]

### 4-Chloro-2-((furan-2-ylmethyl) amino)-5-(piperidin-1-ylsulfonyl) Benzoic Acid (3q, WW096)

According to the General Procedures A and B, the title compound (466 mg, 64% over 2 steps) was obtained as a white solid.

¹H NMR (300 MHz, DMSO-d₆) δ (ppm) 13.49 (s, 1H), 8.73 (s, 1H), 8.33 (s, 1H), 7.63 (s, 1H), 7.10 (s, 1H), 6.49-6.32 (m, 2H), 4.59 (d, J=5.7 Hz, 2H), 3.06 (s, 4H), 1.66-1.30 (m, 6H); HRMS (ESI, m/z) Calculated for C₁₇H₁₈ClN₂O₅S [M−H]⁻: 397.0625, found: 397.0623.

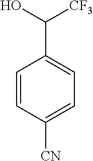 [SMILES: N#Cc1ccc(C(O)C(F)(F)F)cc1]

### 4-(2,2,2-Trifluoro-1-hydroxyethyl)benzonitrile (Intermediate 4)

4-Formylbenzonitrile (3.52 g, 26.84 mmol, 1 eq.) was dissolved in anhydrous THF (15 mL) and cooled to −20° C. To this, TMSCF₃ (6.4 mL, 32.21 mmol, 1.2 eq.) and TBAF (5.4 mL, 5.37 mmol, 0.2 eq.) were added, slowly. The mixture was stirred for 3 h followed by the addition of 1 mL of 1 M HCl solution. The reaction was diluted with EtOAc/H₂O and layers separated. The organic layer was dried over Na₂SO₄, filtered, solvent removed under reduced pressure and subjected to column chromatography (hexanes-EtOAc 3:1) to obtain the title compound (0.39 g, 73%) as a yellow solid. ¹H NMR (300 MHz, CDCl₃) δ (ppm) 7.74 (d, J=8.5 Hz, 2H), 7.64 (d, J=7.7 Hz, 2H), 5.14 (p, J=6.4 Hz, 1H), 2.73 (d, J=4.3 Hz, 1H).

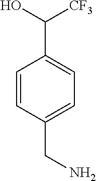 [SMILES: NCc1ccc(C(O)C(F)(F)F)cc1]

### 1-(4-(Aminomethyl)phenyl)-2,2,2-trifluoroethan-1-ol (Intermediate 5)

Lithium aluminium hydride (0.045 g, 7.46 mmol, 1.5 eq.) was suspended in anhydrous THF (10 mL) and cooled to 0° C. The reaction mixture was purged by Ar, before dropwise addition of 4-(2,2,2-trifluoro-1-hydroxyethyl)benzonitrile (1.0 g, 4.97 mmol, 1 M in THF). The reaction was stirred for 2 h and quenched by addition of EtOAc/satd Na₂CO₃ solution. L-tartrate tetrahydrate solution was added to decompose Al(OH)₃ and the reaction was stirred for 16 h. The organic layer was extracted, dried over Na₂SO₄, filtered, solvent removed under reduced pressure. Flash chromatography (CH₂Cl₂-MeOH 80:20) gave the title compound (580 mg, 57%) as a white solid.

Synthesis and Characterisation of Compounds of Formula II

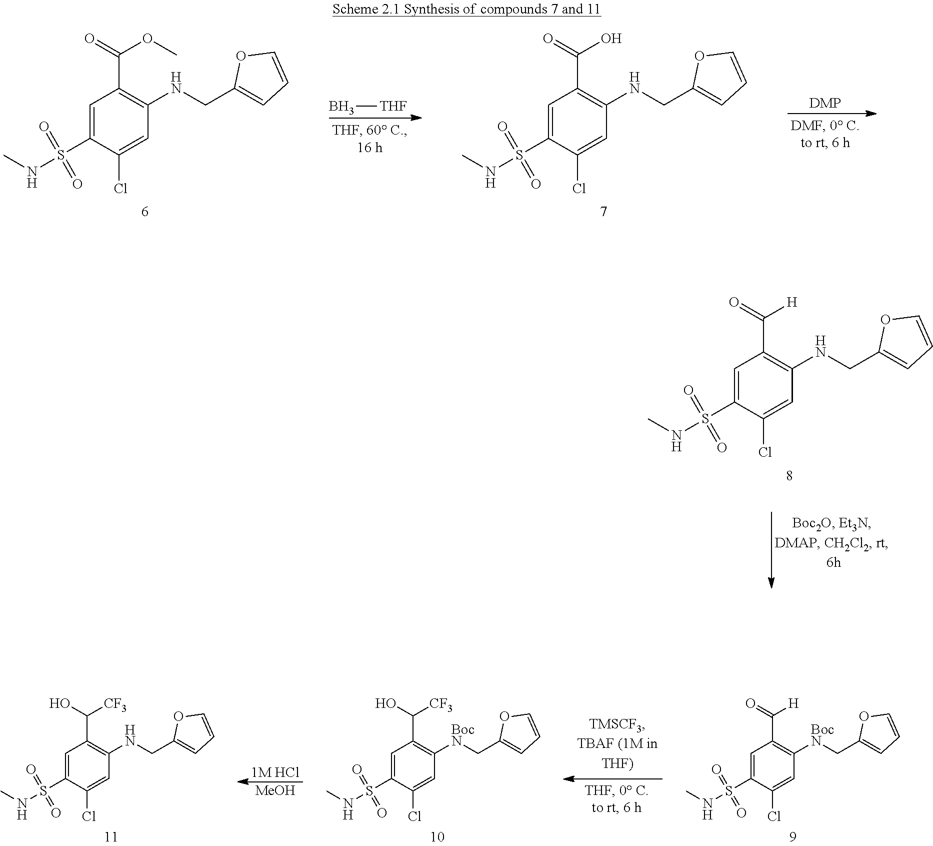

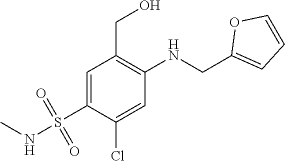 [SMILES: CNS(=O)(=O)c1cc(CO)c(NCc2ccco2)cc1Cl]

### 2-Chloro-4-((furan-2-ylmethyl)amino)-5-(hydroxymethyl)-N-methylbenzenesulfonamide (7, WW109)

Carboxylic acid 6 (100 mg, 0.29 mmol, 1 eq.) was dissolved in anhydrous THF (3 mL). To this, a solution of BH₃-THF (1 M in THF, 0.87 mL, 0.87 mmol, 3 eq.) was added and stirred overnight at 60° C. The reaction was monitored using TLC. The reaction was diluted with EtOAc and washed with standard NaHCO₃ solution. The organic layer was dried over Na₂SO₄, filtered, and solvent removed under reduced pressure. Flash chromatography (hexanes-EtOAc 70:30) gave the title compound (77 mg, 80%) as a white solid.

R_{f} 0.2 (hexanes-EtOAc 65:35); ¹H NMR (300 MHz, CDCl₃) δ (ppm) 7.74 (s, 1H), 7.41 (dd, J₁=1.9 Hz, J₂=0.9 Hz, 1H), 6.80 (s, 1H), 6.33 (m, 1H), 5.92 (s, 1H), 4.76 (d, J=5.5 Hz, 1H), 4.71 (s, 2H), 4.41 (d, J=5.6 Hz, 2H), 2.60 (d, J=5.5 Hz, 3H), 1.74 (s, 1H). HRMS (ESI, m/z) Calculated for C₁₃H₁₅ClN₂O₄SNa [M+Na]⁺: 353.0339, found: 353.0341.

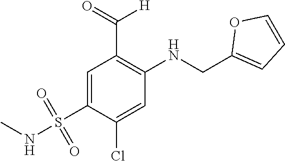 [SMILES: CNS(=O)(=O)c1cc(C=O)c(NCc2ccco2)cc1Cl]

### 2-Chloro-5-formyl-4-((furan-2-ylmethyl)amino)-N-methylbenzenesulfonamide (Intermediate 8)

2-Chloro-4-((furan-2-ylmethyl)amino)-5-(hydroxymethyl)-N-methylbenzenesulfonamide (77 mg, 0.233 mmol, 1 eq.) was dissolved in DMF (2 mL) at 0° C. To this, Dess-Martin periodinane (118.5 mg, 0.279 mmol, 1 eq.) was added, portion-wise and the reaction was stirred for 6 h, warming to room temperature. The reaction was monitored by TLC and when the starting material was consumed, the reaction was poured into EtOAc, washed with 1:1 10% Na₂S₂O₃/satd NaHCO₃. The organic layer was dried over Na₂SO₄, filtered, and solvent removed under reduced pressure. Flash chromatography (hexanes-EtOAc 70:30) afforded the title compound (50 mg, 66%) as a white solid. R_{f} 0.4 (hexanes-EtOAc 7:3).

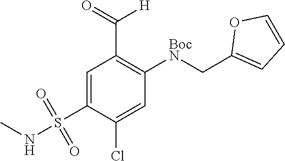 [SMILES: CNS(=O)(=O)c1cc(C=O)c(N(Cc2ccco2)C(=O)OC(C)(C)C)cc1Cl]

### tert-Butyl (5-chloro-2-formyl-4-(N-methylsulfamoyl)phenyl)(furan-2-ylmethyl)carbamate (Intermediate 9)

Aldehyde 8 (28 mg, 0.085 mmol, 1 eq.) was dissolved in anhydrous CH₂Cl₂ (2 mL) and cooled to 0° C. To this, triethylamine (13 mg, 0.128 mmol, 1.5 eq.), Boc₂O (37 mg, 0.170 mmol, 2 eq.), and DMAP (10 mg, 0.085 mmol, 1 eq.), were added. The reaction was stirred for 3 h, allowing to warm to room temperature. The reaction was diluted with CH₂Cl₂ and washed with brine. The organic layer was dried over Na₂SO₄, filtered, solvent removed under reduced pressure. Flash chromatography (hexanes-EtOAc 7:3) gave the title compound (37 mg, 99%) as a yellow solid.

¹H NMR (300 MHz, CDCl₃) δ (ppm) 9.84 (d, J=0.7 Hz, 1H), 9.15 (t, J=5.5 Hz, 1H), 8.33 (s, 1H), 7.41 (dd, J=1.9, 0.8 Hz, 1H), 6.90 (s, 1H), 6.37 (dd, J=3.3, 1.9 Hz, 1H), 6.30 (dd, J=3.2, 0.9 Hz, 1H), 4.49 (d, J=5.7 Hz, 2H), 3.39 (s, 3H), 1.34 (s, 9H).

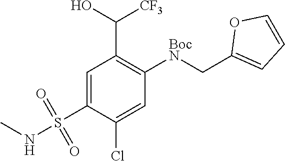 [SMILES: CNS(=O)(=O)c1cc(C(O)C(F)(F)F)c(N(Cc2ccco2)C(=O)OC(C)(C)C)cc1Cl]

### tert-Butyl (5-chloro-4-(N-methylsulfamoyl)-2-(2,2,2-trifluoro-1-hydroxyethyl)phenyl)(furan-2-ylmethyl)carbamate (Intermediate 10)

Aldehyde 9 (36.5 mg, 0.085 mmol, 1 eq.) was dissolved in anhydrous THF (3 mL) and the mixture cooled to 0° C. To this, TMSCF₃ (0.04 mL, 0.17 mmol, 2 eq.) and TBAF (1M in THF, 0.04 mL, 0.043 mmol, 0.5 eq.) were added, dropwise. The mixture was left to stir overnight, allowing to warm to room temperature. When TLC indicated consumption of aldehyde 9, water (1 mL) was added to the reaction and stirred for 1 h. The reaction was diluted with EtOAc and washed with brine. The organic layer was dried over Na₂SO₄, filtered, solvent removed under reduced pressure. Flash chromatography (hexanes-EtOAc 85:15) gave the title compound (21 mg, 50%) as a white solid. R_{f} 0.25 (hexane-EtOAc 7:3).

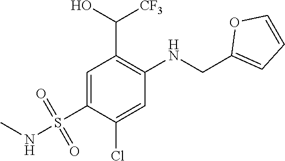 [SMILES: CNS(=O)(=O)c1cc(C(O)C(F)(F)F)c(NCc2ccco2)cc1Cl]

### 2-chloro-4-((furan-2-ylmethyl)amino)-N-methyl-5-(2,2,2-trifluoro-1-hydroxyethyl)benzene Sulfonamide (11, WW094)

tert-Butyl (5-chloro-4-(N-methylsulfamoyl)-2-(2,2,2-trifluoro-1-hydroxyethyl)phenyl)(furan-2-ylmethyl)carbamate (21 mg, 0.042 mmol) was dissolved in MeOH (2 mL) and 5 M HCl (2 mL) was added. The reaction was stirred at room temperature for 1 h before being diluted with EtOAc/H₂O. The organic layers were combined, washed by brine, dried over Na₂SO₄, and concentrated under reduced pressure. Column chromatography gave the title product (16 mg, 93%) as a white solid.

¹H NMR (300 MHz, CDCl₃) δ (ppm) 7.71 (s, 1H), 7.41 (dd, J₁=1.9 Hz, J₂=0.9 Hz, 1H), 6.78 (s, 1H), 6.39-6.24 (m, 3H), 5.10 (q, J=7.3 Hz, 1H), 4.72 (t, J=5.4 Hz, 1H), 4.39 (d, J=5.6 Hz, 2H), 3.56 (s, 1H), 2.59 (d, J=5.4 Hz, 3H); HRMS (ESI, m/z) Calculated for [M−H]⁻: 397.0237, found: 397.0238.

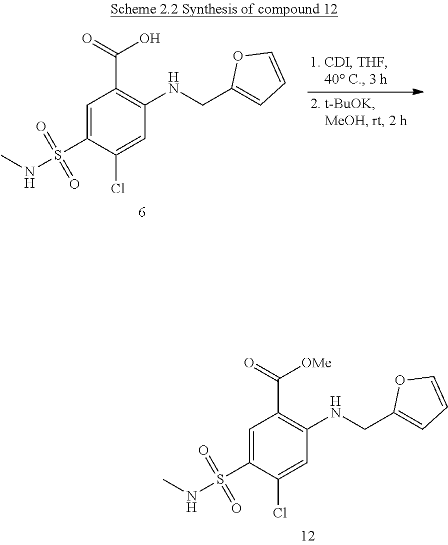 [SMILES: CNS(=O)(=O)c1cc(C(=O)O)c(NCc2ccco2)cc1Cl ; CNS(=O)(=O)c1cc(C(=O)OC)c(NCc2ccco2)cc1Cl]

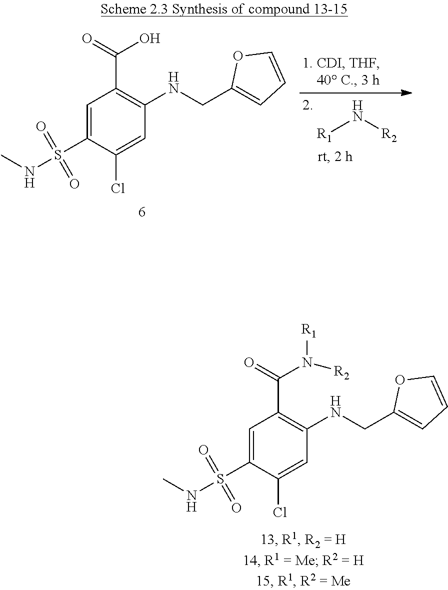

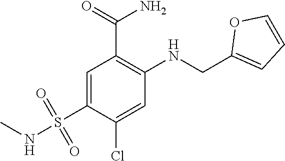 [SMILES: CNS(=O)(=O)c1cc(C(N)=O)c(NCc2ccco2)cc1Cl]

### 4-Chloro-2-((furan-2-ylmethyl)amino)-5-(N-methylsulfamoyl)benzamide (13, WW101)

4-Chloro-2-((furan-2-ylmethyl)amino)-5-(N-methylsulfamoyl)benzoic acid 6 (800 mg, 2.3202 mmol, 1 eq.) and CDI (413.5 mg, 2.5522 mmol, 1.1 eq.) were dissolved in anhydrous THF (8 mL), under Ar. The mixture was stirred at 40° C. and a yellow homogenous solution formed after 3 hours. The 28% NH₄OH solution (0.5 mL, 2.784 mmol, 1.2 eq.) was then added to the mixture at room temperature and stirred for a further 2 hours. The mixture was diluted with EtOAc and washed with saturated NaHCO₃. The organic layer and aq layer were separated, and aq layer extracted with further EtOAc. The organic extracts were combined and dried with Na₂SO₄, and the solvent was removed under reduced pressure. Flash chromatography (hexanes-EtOAc 70:30) gave the title compound (679 mg, 85%) as a white solid.

¹H NMR (300 MHz, DMSO-d₆) δ (ppm) 8.89 (t, J=5.8 Hz, 1H), 8.21 (s, 1H), 8.09 (s, 1H), 7.62 (d, J=18 Hz, 1H), 7.46 (s, 1H), 7.23 (q, J=4.9 Hz, 1H), 6.98 (s, 1H), 6.46-6.32 (m, 2H), 4.50 (d, J=5.8 Hz, 2H), 2.39 (d, J=4.9 Hz, 4H); HRMS (ESI, m/z) Calculated for [M−H]⁻: 342.0315, found: 342.0315.

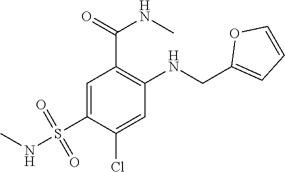 [SMILES: CNC(=O)c1cc(S(=O)(=O)NC)c(Cl)cc1NCc1ccco1]

### 4-Chloro-2-((furan-2-ylmethyl)amino)-N-methyl-5-(N-methylsulfamoyl)benzamide (14, WW102)

4-Chloro-2-((furan-2-ylmethyl)amino)-5-(N-methylsulfamoyl)benzoic acid 6 (200 mg, 0.58 mmol, 1 eq.) and CDI (103.4 mg, 0.638 mmol, 1.1 eq.) were dissolved in THF (2 mL), under Ar. The mixture was stirred at 40° C. and a yellow homogenous solution formed after 3 h. Methylamine solution (0.35 mL, 2M in THF, 1.2 eq.) was then added to the mixture at room temperature and stirred for a further 2 h. The mixture was diluted with EtOAc and washed with saturated NaHCO₃. The organic layer and aq layer were separated and aq layer extracted with further EtOAc. The organic extracts were combined and dried with Na₂SO₄, and the solvent was removed under reduced pressure. Flash chromatography (hexanes-EtOAc 3:1) gave the title compound (51 mg, 25%) as a white solid.

¹H NMR (300 MHz, CDCl₃) δ (ppm) 8.86 (s, 1H), 8.04 (s, 1H), 7.44-7.37 (m, 1H), 6.85 (s, 1H), 6.39-6.25 (m, 3H), 4.77 (q, J=5.5 Hz, 1H), 4.41 (d, J=5.6 Hz, 2H), 2.98 (d, J=4.8 Hz, 3H), 2.61 (d, J=5.4 Hz, 3H); HRMS (ESI, m/z) Calculated for [M+H]⁺: 358.0628, found: 358.0634.

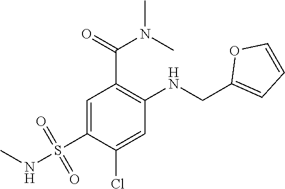 [SMILES: CNS(=O)(=O)c1cc(C(=O)N(C)C)c(NCc2ccco2)cc1Cl]

### 4-Chloro-2-((furan-2-ylmethyl) amino)-N, N-dimethyl-5-(N-methylsulfamoyl) Benzamide (15, WW103)

4-Chloro-2-((furan-2-ylmethyl)amino)-5-(N-methylsulfamoyl)benzoic acid 6 (300 mg, 0.87 mmol, 1 eq.) and CDI (183 mg, 1.13 mmol, 1.2 eq.) were dissolved in anhydrous THF (3 mL), under Ar. The mixture was stirred at 40° C. and a yellow homogenous solution formed after 3 h. The dimethylamine solution (0.6 mL, 2M in THF, 1.2 eq.) was then added to the mixture at room temperature and stirred for a further 2 h. The mixture was diluted with EtOAc and washed with satd NaHCO₃ solution. The organic layer and aq layer were separated and aq layer extracted with further EtOAc. The organic extracts were combined and dried with Na₂SO₄, and the solvent was removed under reduced pressure. Flash chromatography (hexanes-EtOAc 3:1) gave the title compound (237 mg, 73%) as a white solid.

¹H NMR (300 MHz, DMSO-d₆) δ (ppm) 7.84 (s, 1H), 7.39 (dd, J₁=1.9 Hz, J₂=0.8 Hz, 1H), 6.86 (s, 1H), 6.31 (m, 2H), 4.80 (s, 1H), 4.36 (s, 2H), 3.08 (s, 6H), 2.59 (s, 3H); HRMS (ESI, m/z) Calculated for [M−H]⁻: 370.0628, found: 370.0630.

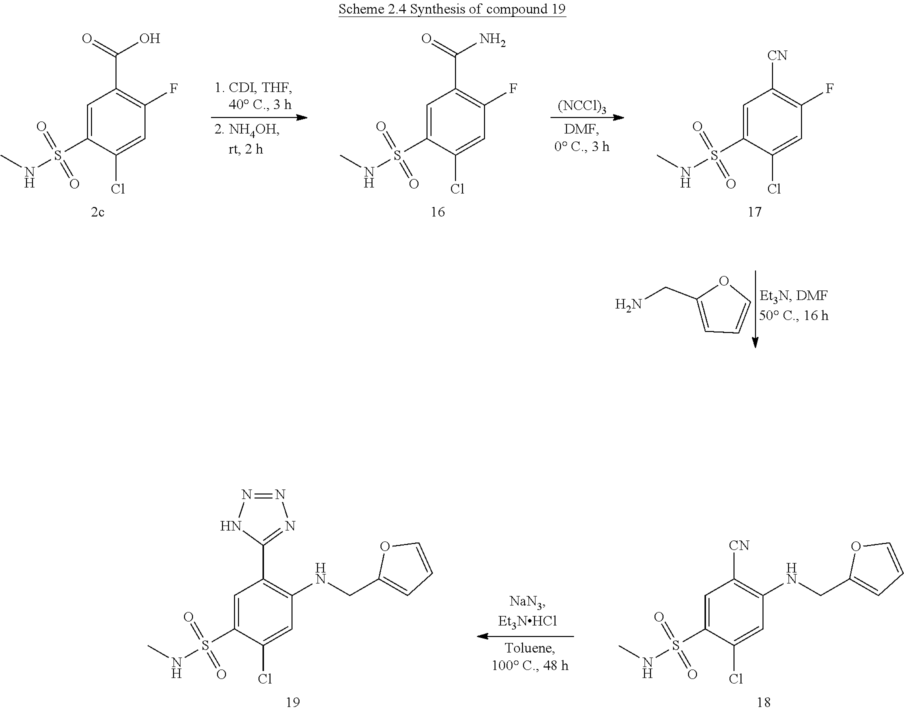

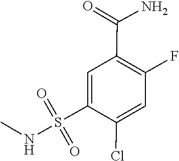 [SMILES: CNS(=O)(=O)c1cc(C(N)=O)c(F)cc1Cl]

### 4-Chloro-2-fluoro-5-(N-methylsulfamoyl)benzamide (Intermediate 16)

Intermediate 2c (800 mg, 3.01 mmol, 1 eq.) and CDI (633 mg, 3.91 mmol, 1.3 eq.) were dissolved in anhydrous THF (9 mL) under Ar. The mixture was stirred at 40° C. and a yellow homogenous solution formed after 3 h. Ammonium hydroxide solution (28%, 0.8 mL, 1.3 eq.) was added at room temperature and stirred for a further 2 h. The mixture was diluted with EtOAc and washed with saturated NaHCO₃ solution. The layers were separated and aq layer was extracted with EtOAc. The organic extracts were combined and dried with Na₂SO₄, and the solvent was removed under reduced pressure. Flash chromatography (CH₂Cl₂-MeOH 95:5) gave the title compound (664 mg, 83%) as a white solid.

¹H NMR (300 MHz, CD₃OD) δ (ppm) 8.46 (d, J=7.6 Hz, 1H), 7.62 (d, J=10.1 Hz, 1H), 2.58 (s, 3H).

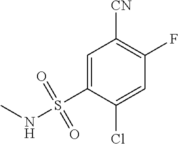

### 2-Chloro-5-cyano-4-fluoro-N-methylbenzenesulfonamide (Intermediate 17)

4-Chloro-2-fluoro-5-(N-methylsulfamoyl)benzamide 16 (50 mg, 0.188 mmol, 1 eq.) was dissolved in anhydrous DMF (1 mL) at 0° C. To this, cyanuric chloride (52 mg, 0.282 mmol, 1.5 eq.) was added and stirred for 3 h, warming to room temperature. The reaction mixture was then diluted with EtOAc/H₂O. The organic layers were combined and washed with brine, dried over Na₂SO₄ and the solvent was removed under reduced pressure. Flash chromatography (hexanes-EtOAc 7:3) gave the title compound (44 mg, 94%) as a white solid.

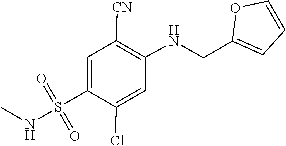 [SMILES: CNS(=O)(=O)c1cc(C#N)c(NCc2ccco2)cc1Cl]

### 2-Chloro-5-cyano-4-((furan-2-ylmethyl)amino)-N-methylbenzenesulfonamide (Intermediate 18)

2-Chloro-5-cyano-4-fluoro-N-methylbenzenesulfonamide 17 (114 mg, 0.460 mmol, 1 eq.) was dissolved in anhydrous DMF (2 mL) at 0° C. To this, furfurylamine (0.06 mL, 0.690 mmol, 1.5 eq.) and triethylamine (0.1 mL, 0.69 mL 1.5 eq.) were added. The reaction mixture was heated to 50° C. for 16 h. The mixture was then diluted with EtOAc/H₂O and layers separated. The pH of the aqueous phase was adjusted to 3 and extracted with EtOAc. The organic layers were washed with brine, dried over Na₂SO₄ and concentrated under reduced pressure. Flash chromatography (hexanes-EtOAc 3:1) gave the title compound (150 mg, 72%) as a white solid.

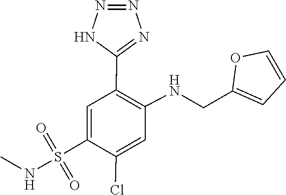 [SMILES: CNS(=O)(=O)c1cc(-c2nnn[nH]2)c(NCc2ccco2)cc1Cl]

### 2-Chloro-4-((furan-2-ylmethyl) amino)-N-methyl-5-((1H-tetrazol-5-yl) benzenesulfonamide (19, WW121)

2-Chloro-5-cyano-4-((furan-2-ylmethyl)amino)-N-methylbenzenesulfonamide 18 (107 mg, 0.328 mmol, 1 eq.), NaN₃ (28 mg, 0.427 mmol, 1.3 eq.) and Et₃N.HCl (59 mg, 0.427 mmol, 1.3 eq.) were dissolved in toluene (2 mL). The reaction was heated to 100° C. and stirred for 48 h. The reaction was then quenched with H₂O and the layers were separated. The aq layer was extracted with EtOAc. The combined organic extracts were washed with brine, dried over Na₂SO₄ and concentrated under reduced pressure. Flash chromatography (hexanes-EtOAc-AcOH 3:1:0.02) gave the title compound (120 mg, 99%) as a white solid.

¹H NMR (300 MHz, DMSO-d₆) δ (ppm) 8.59 (s, 1H), 8.47 (s, 1H), 7.64 (s, 1H), 7.41 (d, J=5.1 Hz, 1H), 7.21 (s, 1H), 6.43 (s, 2H), 4.69 (d, J=4.8 Hz, 2H), 2.43 (d, J=4.9 Hz, 3H); HRMS (ESI, m/z) Calculated for C₁₃H₁₂ClN₆O₃S [M−H]⁻: 367.0380, found: 367.0381.

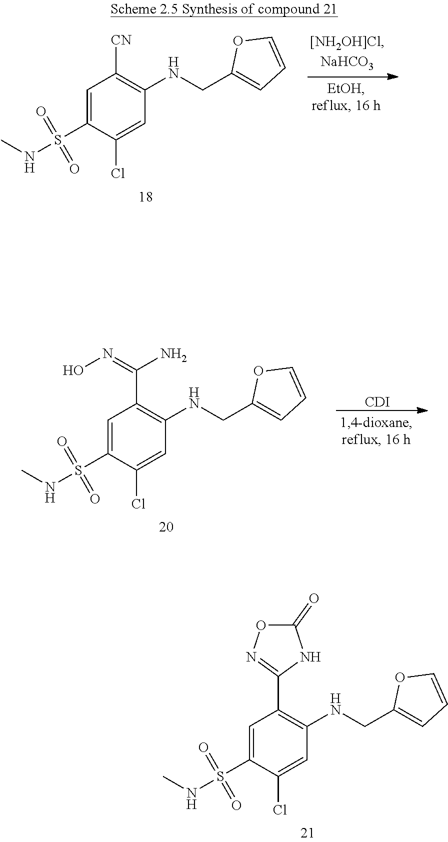

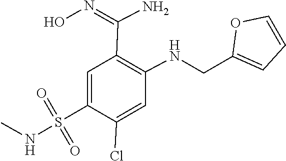 [SMILES: CNS(=O)(=O)c1cc(/C(N)=N\O)c(NCc2ccco2)cc1Cl]

### 4-Chloro-2-((furan-2-ylmethyl)amino)-N′-hydroxy-5-(N-methylsulfamoyl)benzimidamide (Intermediate 20)

2-Chloro-5-cyano-4-((furan-2-ylmethyl)amino)-N-methylbenzenesulfonamide 18 (127.2 mg, 0.391 mmol, 1 eq.) was dissolved in EtOH (2 mL). In a separate flask, the hydroxylamine hydrochloride (55 mg, 0.782 mmol, 2 eq.) was dissolved in NaHCO₃ solution (100 mg, 1.2 M, 3 eq.). This solution was then added to the reaction mixture and heated to reflux for 16 h. The reaction mixture was then diluted with EtOAc/H₂O and the layers were separated. The organic layers were combined, dried over Na₂SO₄ and concentrated under reduced pressure. The crude product was used directly in the next step without further purification.

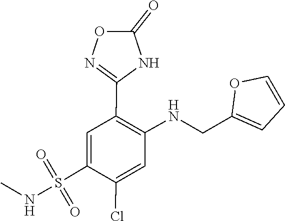 [SMILES: CNS(=O)(=O)c1cc(-c2noc(=O)[nH]2)c(NCc2ccco2)cc1Cl]

### 2-Chloro-4-((furan-2-ylmethyl)amino)-N-methyl-5-(5-oxo-2,5-dihydro-1,2,4-oxadiazol-3-yl)benzenesulfonamide (21, WW187)

To a stirred solution of intermediate 4-chloro-2-((furan-2-ylmethyl)amino)-N′-hydroxy-5-(N-methylsulfamoyl)benzimidamide 20 (64 mg, 0.1882 mmol, 1 eq.) in 1,4-dioxane (1 mL) was added CDI (45.8 mg, 0.282 mmol, 1.5 eq.) and DBU (31.5 mg, 0.207 mmol, 1.1 eq.). The reaction was heated to reflux for 16 h before being diluted with EtOAc/H₂O. The layers were separated and the pH of aq layer was adjusted to 5. The aq layer was then extracted with EtOAc and the organic layers were combined, washed with brine, dried over Na₂SO₄ and concentrated under reduced pressure. Flash chromatography (CH₂Cl₂-MeOH 95:5) gave the title compound (62 mg, 86%) as a white solid.

¹H NMR (300 MHz, DMSO-d6 δ (ppm) 13.23 (s, 1H), 8.18-8.11 (m, 1H), 7.63 (d, J=1.4 Hz, 2H), 7.44-7.34 (m, 1H), 7.16 (s, 1H), 6.46-6.36 (m, 2H), 4.63 (d, J=5.6 Hz, 2H), 2.41 (d, J=4.9 Hz, 3H); HRMS (ESI, m/z) Calculated for C₁₄H₁₄ClN₄O₅S [M+H]⁺: 383.0217, found: 383.0211.

Synthesis and Characterisation of Compounds of Formula III

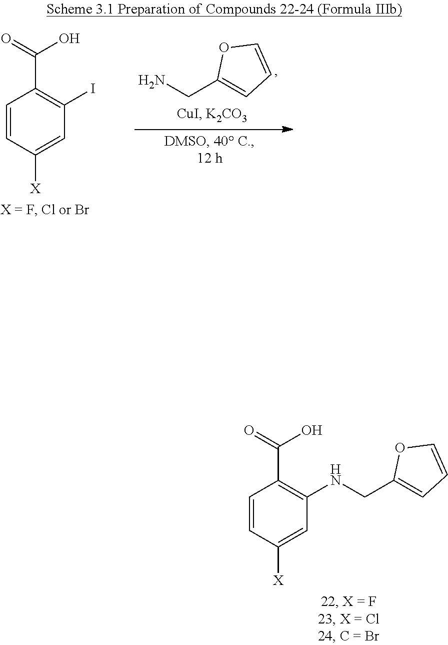

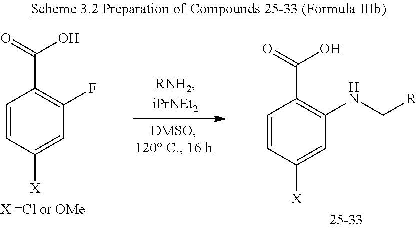

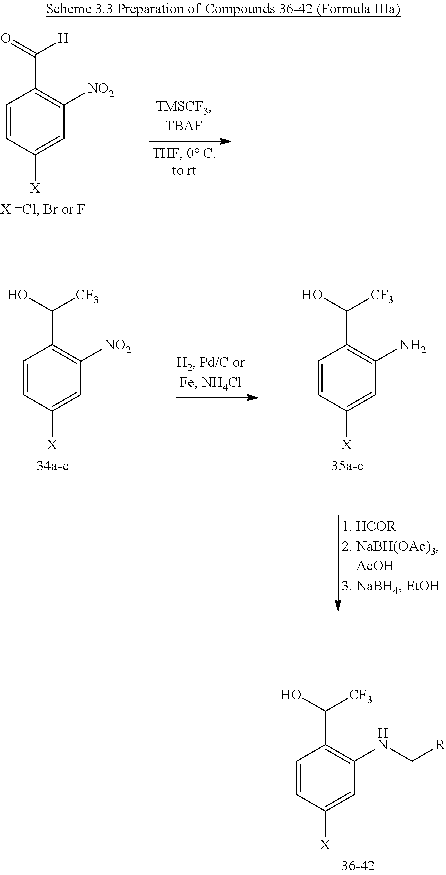

General Procedure C for Synthesis of Compounds 22-24.

The appropriate benzoic acid (1 eq.), furfurylamine (2 eq,), CuI (0.1 eq,), K₂CO₃ (2 eq.) and N,N-dimethylglycein (0.2 eq.) were dissolved in DMSO (0.3 M). The reaction was heated to 40° C. for 12 h before being diluted with EtOAc/1 M HCl solution. The layers were separated and the pH of aq layer was adjusted to 3. The aq layer was then extracted with EtOAc and the organic layers were combined, washed with brine, dried over Na₂SO₄, and concentrated under reduced pressure. Column chromatography gave the desired compounds 22-25 (34-71%).

General Procedure D for Synthesis of Compounds 25-33.

2-Fluoro-4-chlorobenzoic acid or 2-fluoro-4-methoxybenzoic acid (1 eq.) was dissolved in DMSO (0.2 M) under argon. The corresponding amine (1.5 eq.) and N,N-diisopropylethylamine (3 eq.) were added to the reaction mixture. The reaction was heated to 120° C. and stirred for 16 h. The reaction was allowed to cool to rt before diluting with EtOAc and water. The aqueous layer was acidified with 1M HCl and extracted with EtOAc (×3). The combined organic layers were washed with water, dried over Na₂SO₄, filtered, the solvent removed under reduced pressure and purified through column chromatography (hexanes-EtOAc-AcOH 80:19:1) to obtain the desired products (5-36%).

General Procedure E for Synthesis of Compounds 34a-34d.

The appropriate benzaldehyde (1 eq.) was dissolved in anhydrous THF (0.3 M) and cooled to −20° C. To this, TMSCF₃ (1.2 eq.) and TBAF (0.2 eq.) were added, slowly. The reaction was allowed to attain room temperature and stirred for 3 h followed by the addition of 1 mL of 1M HCl solution. The mixture was then diluted with EtOAc/H₂O and the layers were separated. The organic layer was dried over Na₂SO₄, filtered, solvent removed under reduced pressure. Column chromatography gave the desired compounds 34a-34b (53-88%).

General Procedure F for Synthesis of Compounds 35a-35d.

Method I (Synthesis of 35a and 35d): Intermediates 35a and 35d (1 eq.) were dissolved in MeOH (0.3 M) and Pd—C (1 mmol starting material with 20-30 mg Pb—C) was added. To this, 15 psi H₂ was applied to the reaction mixture with oscillation shaking for 4 h. The reaction was monitored by TLC. After TLC indicated consumption of starting material, the reaction mixture was filtered through celite and quenched by satd ammonium chloride. The layers were separated and the aqueous phase was extracted with EtOAc. The combined organic extracts were washed with brine, dried over Na₂SO₄ and concentrated under reduced pressure to afford intermediate compounds 35a and 35d (77-86%) as white solids. The crude products were used directly in the next step without further purification.

Method II (Synthesis of 35b and 35c): Fe powder (100 eq.), and NH₄Cl powder (50 eq.) were dissolved in EtOH (0.05 M) and placed in a flask with reflux condenser. The mixture was then heated to reflux. In a separate flask, intermediates 35b and 35c (1 eq.), was dissolved in EtOH (0.1 M). The EtOH solution was then added to the reaction mixture and stirred. The reaction was monitored by TLC. After TLC indicated consumption of starting material, the reaction mixture was filtered and quenched by satd ammonium chloride. The layers were separated and the aq phase was extracted with EtOAc. The combined organic extracts were washed with brine, dried over Na₂SO₄ and concentrated under reduced pressure to afford intermediate compounds 35b and 35c (49-71%) as white solids. The crude products were used directly in the next step without further purification.

General Procedure G for Synthesis of Compounds 36-42.

Anilines 35a-35d (1 eq.) were dissolved in anhydrous 1,2-dichloroethane (0.3 M), under Ar. Following the addition of appropriate aldehyde (2 eq.) and AcOH (0.5 eq.), the reaction was stirred at 60° C. for 1 h. Na(OAc)₃BH (1.5 eq.) was then added to the reaction and stirred for 16 h. The reaction solvent was removed under reduced pressure and the crude residue was resuspended in EtOH. NaBH₄ (4 eq.) was added and stirred at 60° C. for 1 h. The reaction was quenched with EtOAc/H₂O and the layers were separated. The aq phase was extracted with EtOAc. The combined organic extracts were washed with brine, dried over Na₂SO₄, and concentrated under reduced pressure. Column chromatography gave the compounds 36-42 (29-67%).

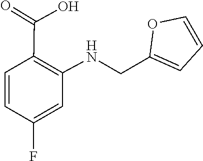 [SMILES: O=C(O)c1ccc(F)cc1NCc1ccco1]

### 4-Fluoro-2-((furan-2-ylmethyl) amino)benzoic Acid (22, WW071)

According to the General Procedure C, the title compound (431 mg, 71%) was obtained as a white solid.

¹H NMR (300 MHz, DMSO-d6) δ (ppm) 12.79 (s, 1H), 8.34 (s, 1H), 7.84 (dd, J₁=8.8 Hz, J₂=7.1 Hz, 1H), 7.66-7.54 (m, 1H), 6.65 (dd, J₁=12.7 Hz, J₂=2.4 Hz, 1H), 6.45-6.32 (m, 3H), 4.45 (d, J=5.1 Hz, 2H); HRMS (ESI, m/z) Calculated for C₁₂H₉FNO₃ [M−H]⁻: 234.0566, found: 234.0567.

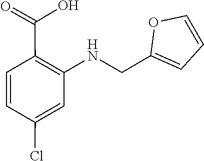 [SMILES: O=C(O)c1ccc(Cl)cc1NCc1ccco1]

### 4-Chloro-2-((furan-2-ylmethyl) amino) Benzoic Acid (23, Compound WW001)

According to the General Procedure C, the title compound (151 mg, 34%) was obtained as a white solid.

¹H NMR (300 MHz, CDCl₃) δ (ppm) 8.05 (s, 1H), 7.92 (d, J=8.6 Hz, 1H), 7.41 (dd, J₁=1.9 Hz, J₂=0.8 Hz, 1H), 6.78 (d, J=2.0 Hz, 1H), 6.64 (dd, J₁=8.6 Hz, J₂=2.0 Hz, 1H), 6.40-6.25 (ddd, J₁=24.3 Hz, J₂=3.2 Hz, J₃=1.4 Hz, 1H), 4.44 (d, J=4.1 Hz, 2H); HRMS (ESI, m/z) Calculated for C₁₂H₉ClNO₃ [M−H]⁻: 250.0271, found 250.0271.

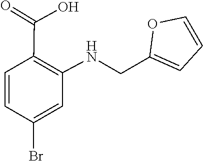 [SMILES: O=C(O)c1ccc(Br)cc1NCc1ccco1]

### 4-Bromo-2-((furan-2-ylmethyl) amino) Benzoic Acid (24, WW073)

According to the General Procedure C, the title compound (188 mg, 46%) was obtained as a white solid.

¹H NMR (300 MHz, DMSO-d6) δ (ppm) 12.96 (s, 1H), 8.25 (s, 1H), 7.69 (d, J=8.5 Hz, 1H), 7.64-7.54 (m, 1H), 7.03 (d, J=1.6 Hz, 1H), 6.74 (dd, J₁=8.5 Hz, J₂=1.6 Hz, 1H), 6.45-6.29 (m, 2H), 4.48 (s, 2H); ESI-HRMS Calculated for Cl₂H₉BrNO₃ [M−H]⁻: 293.9766, found: 293.9766.

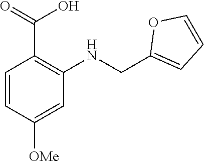 [SMILES: COc1ccc(C(=O)O)c(NCc2ccco2)c1]

### 2-((Furan-2-ylmethyl)amino)-4-methoxybenzoic Acid (25)

According to the General Procedure D, using 2-fluoro-4-methoxybenzoic acid (0.57 g, 3.3 mmol) and 2-furan-2-yl-ethylamine (0.46 mL, mmol), the title compound (91 mg, 5%) was obtained as a white solid.

¹H NMR (300 MHz, Acetone-d₆) δ 8.35 (s, 1H), 7.86 (dd, J=9.0, 0.8 Hz, 1H), 7.50 (dd, J=1.9, 0.9 Hz, 1H), 6.37 (ddd, J=9.0, 3.6, 2.1 Hz, 3H), 6.22 (ddd, J=8.9, 2.5, 0.8 Hz, 1H), 4.48 (d, J=5.4 Hz, 2H), 3.82 (d, J=0.8 Hz, 3H); ¹³C NMR (75 MHz, Acetone-d₆) δ 169.9, 165.6, 142.7, 134.4, 110.9, 107.6, 104.1, 102.9, 95.9, 55.2, 40.1. ESI-HRMS Calculated for C₁₃H₁₄NO₄ [M−H]⁺: 248.0923 found: 248.0928. Purity by HPLC: 99.0% (t_{R}=10.9 min).

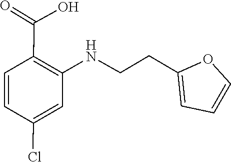 [SMILES: O=C(O)c1ccc(Cl)cc1NCCc1ccco1]

### 4-Chloro-2-((2-(furan-2-yl)ethyl)amino)benzoic Acid (26)

According to the General Procedure D, using 2-fluoro-4-chlorobenzoic acid (0.1 g, 0.57 mmol) and 2-furan-2-yl-ethylamine (0.096 g, 0.86 mmol), the title compound (54 mg, 36%) was obtained as a white solid.

R_{f} 0.29 (hexanes-EtOAc 50:50); ¹H NMR (300 MHz, Acetone-d₆) δ 8.11 (s, 1H), 7.88 (d, J=8.5 Hz, 1H), 7.44 (d, J=1.9 Hz, 1H), 6.79 (d, J=2.0 Hz, 1H), 6.58 (dd, J=8.5, 2.0 Hz, 1H), 6.33 (dd, J=3.2, 1.9 Hz, 1H), 6.20 (d, J=3.2 Hz, 1H), 3.53 (d, J=7.2 Hz, 2H), 3.00 (t, J=6.8 Hz, 2H); ¹³C NMR (75 MHz, Acetone) a 169.8, 153.9, 152.9, 142.4, 141.2, 134.4, 115.2, 111.4, 111.1, 109.4, 107.2, 42.0, 28.3; ESI-HRMS calcd for C₁₃H₁₁ClNO₃ 264.0427, found m/z 264.0431 [M−H]⁻; Purity by HPLC: 98.93% (t_{R}=12.05 min).

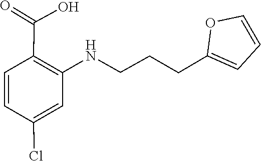 [SMILES: O=C(O)c1ccc(Cl)cc1NCCCc1ccco1]

### 4-Chloro-2-((3-(furan-2-yl)propyl)amino)benzoic Acid (27)

According to the General Procedure D, using 2-fluoro-4-chlorobenzoic acid (0.1 g, 0.57 mmol) and 2-(2-furyl)-1-propanamine (0.11 g, 0.86 mmol), the title compound (48 mg, 30%) was obtained as an off-white solid.

R_{f} 0.38 (hexanes-EtOAc 50:50); ¹H NMR (300 MHz, Acetone-d₆) δ 8.09 (s, 1H), 7.89 (d, J=8.5 Hz, 1H), 7.42 (d, J=1.9 Hz, 1H), 6.74 (d, J=2.1 Hz, 1H), 6.57 (dd, J=8.5, 2.0 Hz, 1H), 6.32 (dd, J=3.2, 1.9 Hz, 1H), 6.11 (d, J=3.2 Hz, 1H), 3.29 (t, J=7.0 Hz, 2H), 2.77 (t, J=7.5 Hz, 2H), 2.03-1.94 (m, 2H); ¹³C NMR (75 MHz, Acetone) δ 170.0, 155.9, 153.1, 142.0, 141.2, 134.5, 115.0, 111.4, 111.0, 109.3, 106.1, 42.5, 28.1, 25.9; ESI-HRMS calcd for C₁₄H₁₃ClNO₃ 278.0584, found m/z 278.0586 [M−H]⁻; Purity by HPLC: 99.74% (t_{R}=12.48 min).

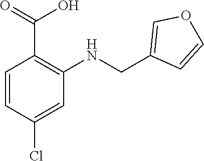 [SMILES: O=C(O)c1ccc(Cl)cc1NCc1ccoc1]

### 4-Chloro-2-((furan-3-ylmethyl)amino)benzoic Acid (28)

According to the General Procedure D, using 2-fluoro-4-chlorobenzoic acid (0.1 g, 0.57 mmol) and 3-furylmethylamine (0.08 mL, 0.86 mmol), the title compound (46 mg, 32%) was obtained as a white solid.

R_{f} 0.33 (hexanes-EtOAc-AcOH 50:49:1); ¹H NMR (300 MHz, Acetone-d₆) δ 8.24 (s, 1H), 7.90 (d, J=8.5 Hz, 1H), 7.61 (dd, J=1.7, 0.9 Hz, 1H), 7.55 (t, J=1.7 Hz, 1H), 6.85 (d, J=2.0 Hz, 1H), 6.61 (dd, J=8.5, 2.0 Hz, 1H), 6.52 (dd, J=1.9, 0.9 Hz, 1H), 4.36 (d, J=4.5 Hz, 2H); ¹³C NMR (75 MHz, Acetone-d₆) δ 169.9, 152.8, 144.6, 141.1, 141.1, 134.4, 123.7, 115.5, 111.8, 111.0, 109.6, 38.5; ESI-HRMS calcd for C₁₂H₁₁ClNO₃ 252.0427, found m/z 252.0422 [M+H]⁺; Purity by HPLC: 100% (t_{R}=11.65 min).

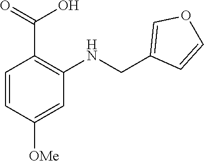 [SMILES: COc1ccc(C(=O)O)c(NCc2ccoc2)c1]

### 2-((Furan-3-ylmethyl)amino)-4-methoxybenzoic Acid (29)

According to the General Procedure D, using 2-fluoro-4-methoxybenzoic acid (0.4 g, 2.16 mmol) and 3-furylmethylamine (0.3 mL, 3.25 mmol), the title compound (60 mg, 12%) was obtained as a white solid.

¹H NMR (300 MHz, Acetone-d₆) δ 8.22 (s, 1H), 7.87 (d, J=8.9 Hz, 1H), 7.60 (t, J=1.1 Hz, 1H), 7.54 (t, J=1.8 Hz, 1H), 6.52 (dd, J=1.8, 0.9 Hz, 1H), 6.30 (d, J=2.4 Hz, 1H), 6.22 (dd, J=8.9, 2.4 Hz, 1H), 4.32 (s, 2H), 3.82 (s, 3H); ¹³C NMR (75 MHz, Acetone) δ 170.2, 165.8, 153.9, 144.3, 140.9, 134.5, 123.9, 110.9, 104.1, 102.8, 95.9, 55.4, 38.3; ESI-HRMS: Calculated for C₁₃H₁₄NO₄ [M−H]⁺: 248.0923; found: 248.0928. Purity by HPLC: 99.5% (t_{R}=10.8 min).

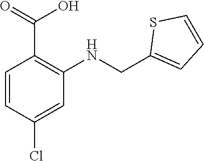 [SMILES: O=C(O)c1ccc(Cl)cc1NCc1cccs1]

### 4-Chloro-2-((thiophen-2-ylmethyl)amino)benzoic Acid (30)

According to the General Procedure D, using 2-fluoro-4-chlorobenzoic acid (0.2 g, 1.15 mmol) and 2-thiophenemethylamine (0.18 mL, 1.72 mmol), the title compound (75 mg, 24%) was obtained as an off-white solid.

R_{f} 0.4 (hexanes-EtOAc-AcOH 50:49:1); ¹H NMR (300 MHz, Acetone-d₆) δ 8.48 (s, 1H), 7.92 (d, J=8.5 Hz, 1H), 7.36 (dd, J=5.1, 1.2 Hz, 1H), 7.12 (dt, J=3.4, 1.1 Hz, 1H), 7.00 (dd, J=5.1, 3.5 Hz, 1H), 6.87 (d, J=2.0 Hz, 1H), 6.63 (dd, J=8.5, 2.0 Hz, 1H), 4.74 (d, J=4.7 Hz, 2H); ¹³C NMR (75 MHz, Acetone) δ 169.8, 152.5, 143.1, 141.0, 134.4, 127.8, 126.2, 125.7, 115.9, 112.1, 109.9, 42.4; ESI-HRMS calcd for C₁₂H₉ClNO₂S 266.0043, found m/z 266.0049 [M−H]⁻; Purity by HPLC: 99.9% (t_{R}=12.01 min).

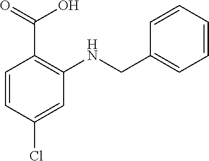 [SMILES: O=C(O)c1ccc(Cl)cc1NCc1ccccc1]

### 2-(Benzylamino)-4-chlorobenzoic Acid (31)

According to the General Procedure D, using 2-fluoro-4-chlorobenzoic acid (0.2 g, 1.15 mmol) and benzylamine (0.19 mL, 1.72 mmol), the title compound (94 mg, 31%) was obtained as a white solid.

R_{f} 0.4 (hexanes-EtOAc-AcOH 50:19:1); 1H NMR (300 MHz, Acetone-d₆) δ 11.17 (s, 1H), 8.47 (s, 1H), 7.92 (d, J=8.5 Hz, 1H), 7.46-7.33 (m, 4H), 7.30-7.23 (m, 1H), 6.74 (d, J=2.0 Hz, 1H), 6.60 (dd, J=8.5, 2.0 Hz, 1H), 4.58-4.45 (m, 2H);

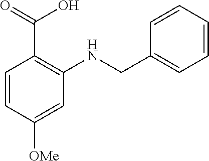 [SMILES: COc1ccc(C(=O)O)c(NCc2ccccc2)c1]

### 2-(Benzylamino)-4-methoxybenzoic Acid (32)

According to the General Procedure D, using 2-fluoro-4-methoxybenzoic acid (0.4 g, 2.16 mmol) and benzylamine (0.54 mL, 5 mmol), the title compound (40 mg, 5%) was obtained as a white solid.

¹H NMR (300 MHz, CDCl₃) δ 8.21 (s, 1H), 7.94 (dd, J=8.9, 1.4 Hz, 1H), 7.40-7.27 (m, 5H), 6.21 (dt, J=8.9, 1.9 Hz, 1H), 6.06 (d, J=2.3 Hz, 1H), 4.47 (s, 2H), 3.73 (d, J=1.4 Hz, 3H); ¹³C NMR (75 MHz, CDCl₃) δ 173.6 (C═O), 165.6 (Cₐᵣ—O), 153.6 (Cₐᵣ—N), 138.7 (Cₐᵣ—CH₂), 134.7 (Cₐᵣ—H), 128.8 (Cₐᵣ—H), 127.3 (Cₐᵣ—H), 127.1 (Cₐᵣ—H), 102.8 (Cₐᵣ—H), 95.7 (Cₐᵣ—COOH), 55.2 (OCH₃), 47.1 (CH₂); ESI-HRMS Calculated for C₁₅H₁₆NO₃ [M+H]⁺ 258.1085, found 258.1151; Purity by HPLC: 98.9% (t_{R}=11.4 min).

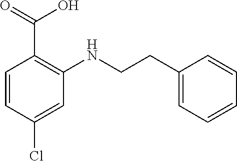 [SMILES: O=C(O)c1ccc(Cl)cc1NCCc1ccccc1]

### 4-Chloro-2-(phenethylamino)benzoic Acid (33)

According to the General Procedure D, using 2-fluoro-4-chlorobenzoic acid (0.2 g, 1.15 mmol) and phenylethylamine (0.22 mL, 1.72 mmol), the title compound (94 mg, 31%) was obtained as a white solid.

R_{f} 0.32 (hexanes-EtOAc-AcOH 50:49:1); ¹H NMR (300 MHz, Acetone-d₆) δ 8.08 (s, 1H), 7.88 (d, J=8.5 Hz, 1H), 7.40-7.27 (m, 4H), 7.21 (td, J=6.4, 5.9, 2.5 Hz, 1H), 6.80 (d, J=2.0 Hz, 1H), 6.58 (dd, J=8.6, 2.0 Hz, 1H), 3.50 (t, J=7.2 Hz, 2H), 2.98 (t, J=7.1 Hz, 2H); ¹³C NMR (75 MHz, Acetone) δ 169.8, 152.9, 141.2, 140.2, 134.4, 129.7, 129.3, 127.2, 115.1, 111.5, 109.3, 44.9, 35.8; ESI-HRMS calcd for C₁₅H₁₅ClNO₂ 276.0791, found m/z 276.0808 [M+H]⁺; Purity by HPLC: 98.3% (t_{R}=12.64 min).

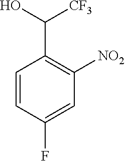 [SMILES: O=[N+]([O-])c1cc(F)ccc1C(O)C(F)(F)F]

### 2,2,2-Trifluoro-1-(4-fluoro-2-nitrophenyl)ethan-1-ol (Intermediate 34a)

According to the General Procedure E, the title compound (945 mg, 88%) was obtained as a yellow solid.

¹H NMR (300 MHz, CDCl₃) δ (ppm) 7.99 (dd, J=8.9, 5.5 Hz, 1H), 7.77 (dd, J=8.2, 2.7 Hz, 1H), 7.46 (ddd, J=9.2, 7.2, 2.7 Hz, 1H), 6.17 (p, J=5.9 Hz, 1H), 2.92 (dt, J=5.4, 1.0 Hz, 1H).

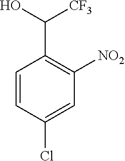 [SMILES: O=[N+]([O-])c1cc(Cl)ccc1C(O)C(F)(F)F]

### 1-(4-Chloro-2-nitrophenyl)-2,2,2-trifluoroethan-1-ol (Intermediate 34b)

According to the General Procedure E, the title compound (145 mg, 53%) was obtained as a yellow solid.

¹H NMR (300 MHz, CDCl₃) δ (ppm) 8.05 (d, J=2.2 Hz, 1H), 7.94 (d, J=8.5 Hz, 1H), 7.72 (dd, J=8.5, 2.2 Hz, 1H), 6.18 (p, J=5.9 Hz, 1H), 2.93 (d, J=5.4 Hz, 1H).

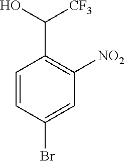 [SMILES: O=[N+]([O-])c1cc(Br)ccc1C(O)C(F)(F)F]

### 1-(4-Bromo-2-nitrophenyl)-2,2,2-trifluoroethan-1-ol (Intermediate 34c)

According to the General Procedure E, the title compound (870 mg, 61%) was obtained as a yellow solid.

¹H NMR (300 MHz, DMSO-d6) δ (ppm) 8.19 (t, J=1.2 Hz, 1H), 7.87 (d, J=1.3 Hz, 2H), 6.16 (p, J=5.9 Hz, 1H), 2.92 (d, J=5.4 Hz, 1H).

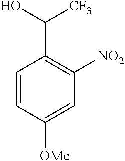 [SMILES: COc1ccc(C(O)C(F)(F)F)c([N+](=O)[O-])c1]

### 2,2,2-Trifluoro-1-(4-methoxy-2-nitrophenyl)ethan-1-ol (Intermediate 34d)

According to the General Procedure E, the title compound (305 mg, 84%) was obtained as a yellow solid.

¹H NMR (300 MHz, CDCl₃) δ (ppm) 7.84 (d, J=8.8 Hz, 1H), 7.52 (d, J=2.7 Hz, 1H), 7.23 (dd, J=8.8, 2.7 Hz, 1H), 6.05 (p, J=6.1 Hz, 1H), 3.90 (s, 3H).

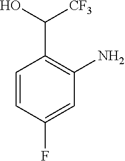 [SMILES: Nc1cc(F)ccc1C(O)C(F)(F)F]

### 1-(2-Amino-4-fluorophenyl)-2,2,2-trifluoroethan-1-ol (Intermediate 35a)

According to the General Procedure F, Method I, the title compound (360 mg, 86%) was obtained as a white solid. R_{f} 0.25 (hexanes-EtOAc 85:15).

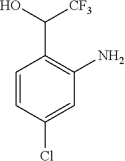 [SMILES: Nc1cc(Cl)ccc1C(O)C(F)(F)F]

### 1-(2-Amino-4-chlorophenyl)-2,2,2-trifluoroethan-1-ol (Intermediate 35b)

According to the General Procedure F, Method II, the title compound (149 mg, 49%) was obtained as a white solid. R_{f} 0.3 (hexane-EtOAc 85:15).

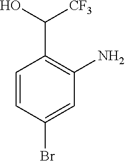 [SMILES: Nc1cc(Br)ccc1C(O)C(F)(F)F]

### 1-(2-Amino-4-bromophenyl)-2,2,2-trifluoroethan-1-ol (Intermediate 35c)

According to the General Procedure F, Method II, the title compound (329 mg, 71%) was obtained as a white solid. R_{f} 0.25 (hexanes-EtOAc 85:15).

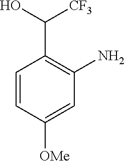 [SMILES: COc1ccc(C(O)C(F)(F)F)c(N)c1]

### 1-(2-Amino-4-methoxyphenyl)-2,2,2-trifluoroethan-1-ol (Intermediate 35d)

According to the General Procedure F, Method I, the title compound (177 mg, 77%) was obtained as a white solid. R_{f} 0.1 (hexane-EtOAc 4:1).

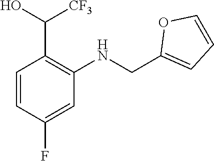 [SMILES: OC(c1ccc(F)cc1NCc1ccco1)C(F)(F)F]

### 2,2,2-Trifluoro-1-(4-fluoro-2-((furan-2-ylmethyl) amino)phenyl) ethan-1-ol (36, WW011)

According to the General Procedure G, the title compound (103 mg, 67%) was obtained as a white solid.

¹H NMR (300 MHz, CDCl₃) δ (ppm) 7.38 (dd, J₁=1.9 Hz, J₂=0.8 Hz, 1H), 7.14 (dd, J₁=8.3 Hz, J₂=6.4 Hz, 1H), 6.53-6.39 (m, 2H), 6.34 (dd, J₁=3.2 Hz, J₂=1.9 Hz, 1H), 6.25 (dd, J₁=3.2 Hz, J₂=0.9 Hz, 1H), 5.22 (s, 1H), 5.06 (q, J=7.4 Hz, 1H), 4.31 (s, 2H), 2.89 (s, 1H); HRMS (ESI, m/z) Calculated for C₁₃H₁₂F₄NO₂ [M+H]⁺: 290.0804, found: 290.0997.

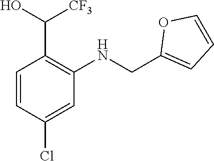 [SMILES: OC(c1ccc(Cl)cc1NCc1ccco1)C(F)(F)F]

### 1-(4-Chloro-2-((furan-2-ylmethyl) amino) phenyl)-2,2,2-trifluoroethan-1-ol (37, WW013)

According to the General Procedure G, the title compound (55 mg, 58%) was obtained as a white solid.

¹H NMR (300 MHz, CDCl₃) δ (ppm) 7.39 (m, 1H), 7.10 (d, J=8.1 Hz, 1H), 6.83-6.68 (m, 2H), 6.34 (dd, J₁=3.3 Hz, J₂=1.9 Hz, 1H), 6.25 (d, J=3.2 Hz, 1H), 5.22-4.92 (m, 2H), 4.32 (s, 2H), 2.95 (s, 1H); HRMS (ESI, m/z) Calculated for C₁₃H₁₂ClF₃NO₂ [M+H]⁺: 306.0509, found: 306.0504.

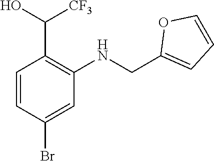 [SMILES: OC(c1ccc(Br)cc1NCc1ccco1)C(F)(F)F]

### 1-(4-Bromo-2-((furan-2-ylmethyl) amino) phenyl)-2,2,2-trifluoroethan-1-ol (38, WW016)

According to the General Procedure G, the title compound (84 mg, 50%) was obtained as a white solid.

¹H NMR (300 MHz, CDCl₃) δ (ppm) 7.39 (s, 1H), 7.04 (d, J=8.1 Hz, 1H), 6.96-6.85 (m, 2H), 6.38-6.21 (m, 2H), 5.21-4.96 (m, 2H), 4.32 (d, J=5.2 Hz, 2H), 2.98 (s, 1H).

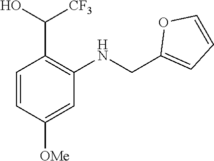 [SMILES: COc1ccc(C(O)C(F)(F)F)c(NCc2ccco2)c1]

### 2,2,2-trifluoro-1-(2-((furan-2-ylmethyl) amino)-4-methoxyphenyl) ethan-1-ol (39, WW003)

According to the General Procedure G, the title compound (32 mg, 29%) was obtained as a white solid.

¹H NMR (300 MHz, DMSO-d6) δ (ppm) 7.56 (dd, Ji'₂ 1 Hz, J₂=1.77 Hz, 1H), 7.16 (d, J=8.2 Hz, 1H), 6.72 (d, J=5.4 Hz, 1H), 6.37 (dd, J₁=3.2 Hz, J₂=1.8 Hz, 1H), 6.24 (d, J=3.1 Hz, 1H), 6.22-6.15 (m, 2H), 6.11 (t, 1H), 5.34-5.16 (m, 1H), 4.29 (d, J=5.8 Hz, 2H), 3.66 (s, 3H); HRMS (ESI, m/z) Calculated for C₁₄H₁₅F₃NO₃ [M+H]⁺: 302.1004, found 302.0989.

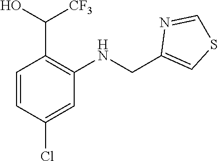 [SMILES: OC(c1ccc(Cl)cc1NCc1cscn1)C(F)(F)F]

### 1-(4-Chloro-2-((thiazol-4-ylmethyl) amino) phenyl)2,2,2-trifluoroethan-1-ol (40, WW021)

According to the General Procedure G, the title compound (65 mg, 65%) was obtained as a white solid.

¹H NMR (300 MHz, CDCl₃) δ (ppm) 8.73 (s, 1H), 7.26 (s, 1H), 7.03 (d, J=8.0 Hz, 1H), 6.68 (d, J=8.9 Hz, 2H), 5.73 (s, 1H), 5.05 (q, J=7.4 Hz, 2H), 4.47 (s, 2H); HRMS (ESI, m/z): Calculated for C₁₂H₉ClF₃N₂OS [M−H]⁻: 321.0076, found: 321.0077.

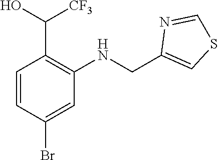 [SMILES: OC(c1ccc(Br)cc1NCc1cscn1)C(F)(F)F]

### 1-(4-Bromo-2-((thiazol-4-ylmethyl) amino) phenyl)-2,2,2-trofluoroethan-1-ol (41, WW017)

According to the General Procedure G, the title compound (41 mg, 50%) was obtained as a white solid.

¹HNMR (300 MHz, DMSO-d6) δ (ppm) 9.08 (d, J=1.9 Hz, 1H), 7.35 (d, J=1.9 Hz, 1H), 7.21 (d, J=8.2 Hz, 1H), 6.95 (d, J=5.3 Hz, 1H), 6.81-6.65 (m, 2H), 6.57 (t, J=5.9 Hz, 1H), 5.38 (p, J=6.9 Hz, 1H), 4.47 (d, J=5.8 Hz, 2H); HRMS (ESI, m/z): Calculated for C₁₂H₉BrF₃N₂OS [M−H]⁻: 364.9571, found: 364.9569.

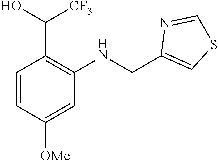 [SMILES: COc1ccc(C(O)C(F)(F)F)c(NCc2cscn2)c1]

### 2,2,2-Trifluoro-1-(4-methoxy-2-(((thiazol-4-ylmethyl) amino) phenyl) ethan-1-ol (42, WW005)

According to the General Procedure G, the title compound (75 mg, 47%) was obtained as a white solid.

¹H NMR (300 MHz, DMSO-d6) δ (ppm) 9.06 (d, J=1.9 Hz, 1H), 7.34 (d, J=1.9 Hz, 1H), 7.15 (d, J=8.5 Hz, 1H), 6.72 (d, J=5.4 Hz, 1H), 6.26 (s, 1H), 6.17 (dd, J₁=8.5 Hz, J₂=2.4 Hz, 1H), 6.07 (d, J=2.4 Hz, 1H), 5.34-5.20 (m, 1H), 4.56-4.29 (m, 1H), 3.61 (s, 3H); HRMS (ESI, m/z) Calculated for C₁₃H₁₄F₃N₂O₂S [M+H]⁺: 319.0728, found 319.0729.

Synthesis and Characterisation of Compounds of Formula IV

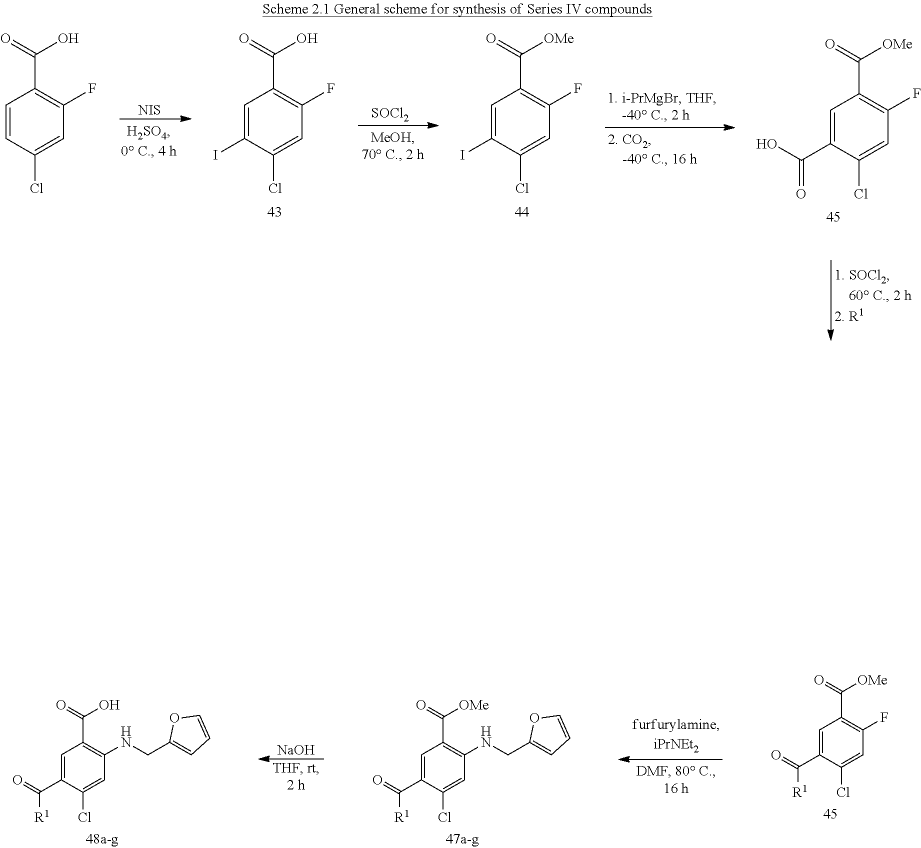

General Procedure H for Preparation of Compounds 46a-46g

Intermediate 45 (1 eq.) was dissolved SOCl₂ (5 eq.) and heated to 60° C. for 3 h. The thionyl chloride was then removed in vacuo and got a yellow solid. The solid was then resuspended in anhydrous THF (0.3 M) and cooled to 0° C. To this, the appropriate amine (1.2 eq.) was added to reaction and stirred at 0° C. for 2 h before quenched by satd NaHCO₃ solution. The layers were then extracted with EtOAc. The organic extracts were combined and washed with brine, dried over Na₂SO₄ and concentrated under reduced pressure. Flash chromatography gave the desired compounds 9a-9g (81-91%) as white solids.

General Procedure I for Preparation of Compounds 47a-47g

Intermediates 46a-46g (1 eq.) were dissolved in anhydrous DMF (0.3 M). To this, furfurylamine (3 eq.) and triethylamine (3 eq.) were added and stirred at 80° C. for 16 h before being diluted with EtOAc/H₂O. The layers were separated and the organic layers were combined. The organic layer was washed with brine, dried over Na₂SO₄ and concentrated under reduced pressure. Flash chromatography gave intermediates 47a-47g (20-73%) as white solids.

General Procedure J for Preparation of Compounds 48a-48g

Intermediates 47a-47g (1 eq.) were dissolved in anhydrous THF (0.3 M). To this, 2 M NaOH was added until the pH was adjusted to 9. The reaction mixture was then stirred at room temperature for 2 h before being diluted with EtOAc. The layers were separated and the pH of aqueous phase was adjusted to 3. The organic layers were combined and washed with brine, dried over Na₂SO₄ and concentrated under reduced pressure. Flash chromatography give compounds 48a-48g (36-93%) as white solids.

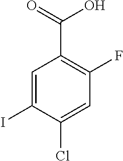 [SMILES: O=C(O)c1cc(I)c(Cl)cc1F]

### 4-Chloro-2-fluoro-5-iodobenzoic Acid (Intermediate 43)

4-Chloro-2-fluorobenzoic acid (5 g, 28.6 mmol, 1 eq.) was dissolved in concentrated sulfuric acid (50 mL) at 0° C. To this, N-iodosuccinimide (7 g, 31.5 mmol, 1.1 eq.) was added, portion-wise. The reaction mixture was stirred at 0° C. at 4 h. The reaction was then warmed up to room temperature and poured onto water (120 mL). 1 N sodium thiosulfate (40 mL) was added. The precipitate was washed with water and dried. The crude residue was resuspended in hot 1:1 ethanol:water. The yellow precipitate was discarded. Additional water was added, and the white precipitate was collected and dried to afford the title product (7.09 g, 83%) as a white solid.

¹H NMR (300 MHz, CDCl₃) δ (ppm) 13.62 (s, 1H), 8.30 (dd, J=7.9, 3.7 Hz, 1H), 7.75 (dd, J=10.6, 3.7 Hz, 1H); HRMS (ESI m/z) Calculated for C₇H₂ClFIO₂ [M−H]⁻: 298.8722, found: 298.8773.

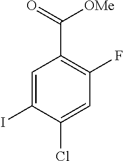 [SMILES: COC(=O)c1cc(I)c(Cl)cc1F]

### Methyl 4-chloro-2-fluoro-5-iodobenzoate (Intermediate 44)

Intermediate 43 (3.9 g, 12.9534 mmol, 1 eq.) was dissolved in MeOH (40 mL). To this, SOCl₂ (5 mL, 5 eq.) was slowly added at 0° C. The reaction mixture was refluxed at 70° C. for 2 h before cooling to room temperature. MeOH was removed in vacuo and the resulting residue was poured onto an ice-water mixture and extracted with EtOAc. The combined organic extracts were washed with satd NaHCO₃ solution, dried over Na₂SO₄, and concentrated under reduced pressure to afford the title product (3.55 g, 87%) as a white solid. The product was directly applied to next steps without purification.

¹H NMR (300 MHz, CDCl₃) δ (ppm) 8.41 (d, J=7.5 Hz, 1H), 7.30 (d, J=10.1 Hz, 1H), 3.94 (s, 3H).

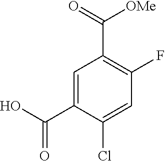 [SMILES: COC(=O)c1cc(C(=O)O)c(Cl)cc1F]

### 2-Chloro-4-fluoro-5-(methoxycarbonyl)benzoic Acid (Intermediate 45)

Intermediate 44 (3.6 g, 1 eq.) was dissolved in anhydrous THF (35 mL) under Ar and cooled to −40° C. i-PrMgBr (18 mL, 0.75 M, 1.2 eq.) was added and stirred at −40° C. for 2 h. The reaction system was then evacuated and purged with dry CO₂ and stirred for another 16 h before being diluted with EtOAc/H₂O. The pH of the aqueous phase was adjusted to 3 and extracted with EtOAc. The organic layers were washed with brine, dried over Na₂SO₄ and concentrated under reduced pressure. Column chromatography (hexanes-EtOAc-AcOH 4:1:0.05) gave the title compound 45 (2.61 g, 82%) as a white solid.

¹H NMR (300 MHz, DMSO-d6) δ (ppm) 13.77 (s, 1H), 8.34 (dd, J=7.9, 1.0 Hz, 1H), 7.76 (d, J=10.7 Hz, 1H), 3.87 (s, 3H); HRMS (ESI, m/z) Calculated for C₉H₅ClFO₄ [M+H]⁺: 230.9860, found: 230.9861.

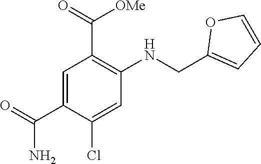 [SMILES: COC(=O)c1cc(C(N)=O)c(Cl)cc1NCc1ccco1]

### Methyl 5-carbamoyl-4-chloro-2-((furan-2-ylmethyl)amino)benzoate (47a)

According to the General Procedures H and I, the title compound (387 mg, 73% over 2 steps) was obtained as a white solid.

¹H NMR (300 MHz, DMSO-d6 δ (ppm) 8.20 (t, J=5.8 Hz, 1H), 7.96 (d, J=1.8 Hz, 1H), 7.69 (s, 1H), 7.61 (d, J=2.1 Hz, 1H), 7.40 (s, 1H), 6.95 (d, J=1.8 Hz, 1H), 6.46-6.31 (m, 2H), 4.54 (d, J=5.8 Hz, 2H), 3.82 (d, J=1.8 Hz, 3H); HRMS (ESI, m/z) Calculated for C₁₄H₁₄O₄N₂Cl [M+H]⁺: 309.0642, found: 309.0640.

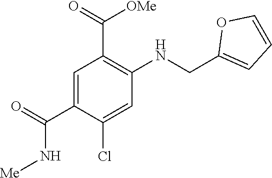 [SMILES: CNC(=O)c1cc(C(=O)OC)c(NCc2ccco2)cc1Cl]

### Methyl 4-chloro-2-((furan-2-ylmethyl)amino)-5-(methylcarbamoyl)benzoate (47b)

According to the General Procedures H and I, the title compound (80 mg, 41% over 2 steps) was obtained as a white solid.

¹H NMR (300 MHz, DMSO-d6 δ (ppm) 8.18 (d, J=5.8 Hz, 2H), 7.89 (s, 1H), 7.61 (d, J=1.8 Hz, 1H), 6.96 (s, 1H), 6.45-6.31 (m, 2H), 4.54 (d, J=5.9 Hz, 2H), 3.81 (s, 3H), 2.70 (d, J=4.6 Hz, 3H); HRMS (ESI, m/z) Calculated for C₁₅H₁₆ClN₂O₄ [M+H]⁺: 323.0799, found: 309.0782.

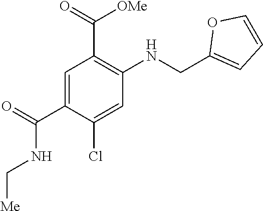 [SMILES: CCNC(=O)c1cc(C(=O)OC)c(NCc2ccco2)cc1Cl]

### Methyl 4-chloro-5-(ethylcarbamoyl)-2-((furan-2-ylmethyl)amino)benzoate (47c)

According to the General Procedures H and I, the title compound (131 mg, 47% over 2 steps) was obtained as a white solid.

¹H NMR (300 MHz, DMSO-d6 δ (ppm) 8.22 (dt, J=17.7, 5.7 Hz, 2H), 7.86 (d, J=0.8 Hz, 1H), 7.68-7.55 (m, 1H), 6.96 (s, 1H), 6.47-6.22 (m, 2H), 4.54 (d, J=5.8 Hz, 2H), 3.82 (s, 3H), 3.28-3.10 (m, 2H), 1.16-1.00 (m, 3H).

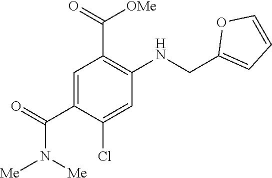 [SMILES: COC(=O)c1cc(C(=O)N(C)C)c(Cl)cc1NCc1ccco1]

### Methyl 4-chloro-5-(dimethylcarbamoyl)-2-((furan-2-ylmethyl)amino)benzoate (47d)

According to the General Procedures H and I, the title compound (86 mg, 20% over 2 steps) was obtained as a white solid.

¹H NMR (300 MHz, DMSO-d6 δ (ppm) 7.63 (dd, J=1.9, 0.9 Hz, 1H), 7.00 (s, 1H), 6.37 (dd, J=3.2, 1.0 Hz, 1H), 4.53 (d, J=5.8 Hz, 2H), 3.80 (s, 3H), 2.96 (s, 3H), 2.79 (s, 3H).

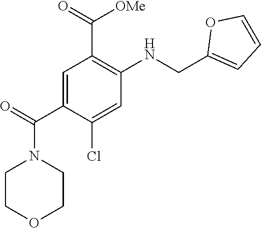 [SMILES: COC(=O)c1cc(C(=O)N2CCOCC2)c(Cl)cc1NCc1ccco1]

### Methyl 4-chloro-2-((furan-2-ylmethyl)amino)-5-(morpholine-4-carbonyl)benzoate (47e)

According to the General Procedures H and I, the title compound (166 mg, 51% over 2 steps) was obtained as a white solid.

¹H NMR (300 MHz, DMSO-d6 δ (ppm) 8.18 (t, J=5.8 Hz, 1H), 7.73 (s, 1H), 7.62 (d, J=2.2 Hz, 1H), 7.00 (s, 1H), 6.46-6.33 (m, 2H), 4.53 (d, J=5.8 Hz, 2H), 3.81 (s, 3H), 3.56 (d, J=28.1 Hz, 6H), 3.18 (s, 2H); HRMS (ESI, m/z) Calculated for C₁₈H₂₀O₅N₂Cl [M+H]⁺: 379.1061, found: 379.1054.

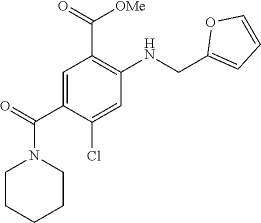 [SMILES: COC(=O)c1cc(C(=O)N2CCCCC2)c(Cl)cc1NCc1ccco1]

### Methyl 4-chloro-2-((furan-2-ylmethyl)amino)-5-(piperidine-1-carbonyl)benzoate (47f)

According to the General Procedures H and I, the title compound (399 mg, 67% over 2 steps) was obtained as a white solid.

¹H NMR (300 MHz, DMSO-d6 δ (ppm) 8.15 (t, J=5.8 Hz, 1H), 7.74-7.57 (m, 2H), 6.99 (s, 1H), 6.49-6.30 (m, 2H), 4.53 (d, J=5.8 Hz, 2H), 3.80 (s, 3H), 3.68-3.43 (m, 2H), 3.13 (d, J=6.2 Hz, 2H), 1.72-1.28 (m, 6H).

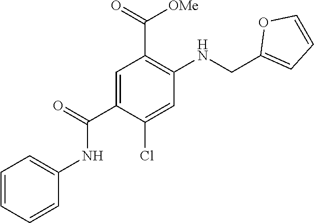 [SMILES: COC(=O)c1cc(C(=O)Nc2ccccc2)c(Cl)cc1NCc1ccco1]

### Methyl 4-chloro-2-((furan-2-ylmethyl)amino)-5-(phenylcarbamoyl)benzoate (47g)

According to the General Procedures H and I, the title compound (438 mg, 66% over 2 steps) was obtained as a white solid.

¹H NMR (300 MHz, DMSO-d6 δ (ppm) 10.32 (s, 1H), 8.29 (t, J=5.9 Hz, 1H), 7.99 (s, 1H), 7.78-7.55 (m, 3H), 7.32 (t, J=7.8 Hz, 2H), 7.17-6.96 (m, 2H), 6.49-6.31 (m, 2H), 4.59 (d, J=5.9 Hz, 2H), 3.83 (s, 3H). HRMS (ESI, m/z) Calculated for C₂₀H₁₈O₄N₂Cl [M+H]⁺: 385.0955, found: 385.0949.

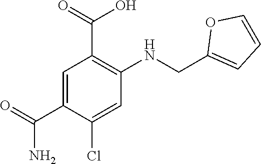 [SMILES: NC(=O)c1cc(C(=O)O)c(NCc2ccco2)cc1Cl]

### 5-Carbamoyl-4-chloro-2((furan-2-ylmethyl)amino) Benzoic Acid (48a)

According to the General Procedure J, the title compound (35 mg, 93%) was obtained as a white solid.

¹H NMR (300 MHz, DMSO-d6 δ (ppm) 13.16 (s, 1H), 8.40 (s, 1H), 7.95 (s, 1H), 7.70-7.57 (m, 2H), 7.35 (s, 1H), 6.90 (s, 1H), 6.45-6.30 (m, 2H), 4.52 (d, J=5.3 Hz, 2H). HRMS (ESI, m/z) Calculated for C₁₃H₁₁ClN₂O₄ [M+H]⁺: 295.0486, found: 295.0477.

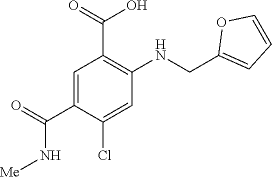 [SMILES: CNC(=O)c1cc(C(=O)O)c(NCc2ccco2)cc1Cl]

### 4-Chloro-2-((furan-2-ylmethyl)amino)-5-(methylcarbamoyl)benzoic Acid (48b)

According to the General Procedure J, the title compound (108 mg, 70%) was obtained as a white solid.

¹H NMR (300 MHz, DMSO-d6 δ (ppm) 13.17 (s, 1H), 8.40 (s, 1H), 8.15 (d, J=4.8 Hz, 1H), 7.88 (s, 1H), 7.65-7.54 (m, 1H), 6.91 (s, 1H), 6.41 (dd, J=3.2 Hz, 1.9 Hz, 1H), 6.34 (d, J=3.3 Hz, 1H), 4.52 (d, J=5.1 Hz, 2H), 2.70 (d, J=4.6 Hz, 3H); HRMS (ESI, m/z) Calculated for C₁₄H₁₂ClN₂O₄ [M−H]⁻: 307.0486, found: 307.0482.

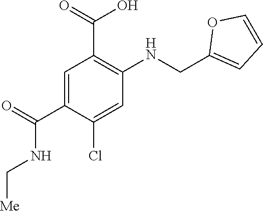 [SMILES: CCNC(=O)c1cc(C(=O)O)c(NCc2ccco2)cc1Cl]

### 4-Chloro-5-(ethylcarbamoyl)-2((furan-2-ylmethyl)amino)benzoic Acid (48c)

According to the General Procedure J, the title compound (40 mg, 36%) was obtained as a white solid.

¹H NMR (300 MHz, DMSO-d6 δ (ppm) 13.17 (s, 1H), 8.41 (s, 1H), 8.21 (d, J=6.0 Hz, 1H), 7.85 (s, 1H), 7.61 (d, J=1.8 Hz, 1H), 6.90 (s, 1H), 6.41 (dd, J=3.2, 1.8 Hz, 1H), 6.34 (d, J=3.3 Hz, 1H), 4.52 (d, J=5.1 Hz, 2H), 3.27-3.11 (m, 2H), 1.07 (t, J=7.2 Hz, 3H); HRMS (ESI, m/z) Calculated for C₁₅H₁₄ClN₂O₄ [M−H]⁻: 321.0642, found: 321.0641.

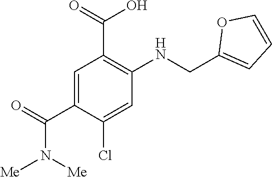 [SMILES: CN(C)C(=O)c1cc(C(=O)O)c(NCc2ccco2)cc1Cl]

### 4-Chloro-5-(dimethylcarbamoyl)-2((furan-2-ylmethyl)amino)benzoic Acid (48d)

According to the General Procedure J, the title compound (25 mg, 37%) was obtained as a white solid.

¹H NMR (300 MHz, DMSO-d6 δ (ppm) 13.17 (s, 1H), 7.67 (s, 1H), 7.62 (d, J=17.7 Hz, 1H), 6.95 (s, 1H), 6.45-6.33 (m, 2H), 4.50 (s, 2H), 2.95 (s, 3H), 2.79 (s, 3H); HRMS (ESI, m/z) Calculated for C₁₅H₁₄ClN₂O₄ [M−H]⁻: 321.0642, found: 321.0650.

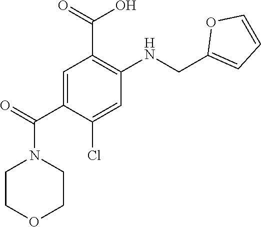 [SMILES: O=C(O)c1cc(C(=O)N2CCOCC2)c(Cl)cc1NCc1ccco1]

### 4-Chloro-2-((furan-2-ylmethyl)amino)-5-(morpholine-4-carbonyl)benzoic acid (48e)

According to the General Procedure J, the title compound (80 mg, 62%) was obtained as a white solid.

¹H NMR (300 MHz, DMSO-d6 δ (ppm) 13.20 (s, 1H), 8.36 (s, 1H), 7.71 (d, J=1.4 Hz, 1H), 7.62 (d, J=2.3 Hz, 1H), 6.96 (d, J=1.4 Hz, 1H), 6.46-6.32 (m, 2H), 4.51 (d, J=5.1 Hz, 2H), 3.33 (s, 1H), 3.18 (s, 2H); HRMS (ESI, m/z) Calculated for C₁₇H₁₈ClN₂O₅[M+H]⁺: 365.0904, found: 365.0911.

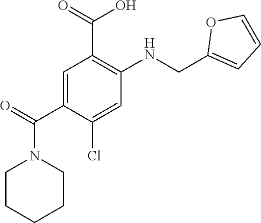 [SMILES: O=C(O)c1cc(C(=O)N2CCCCC2)c(Cl)cc1NCc1ccco1]

### 4-Chloro-2-((furan-2-ylmethyl)amino)-5-(piperidine-1-carbonyl)benzoic Acid (48f)

According to the General Procedure J, the title compound (15 mg, 44%) was obtained as a white solid.

¹H NMR (300 MHz, DMSO-d6 δ (ppm) 13.18 (s, 1H), 8.32 (s, 1H), 7.69-7.58 (m, 2H), 6.95 (d, J=1.3 Hz, 1H), 6.46-6.32 (m, 2H), 4.51 (d, J=4.9 Hz, 2H), 3.59 (s, 1H), 3.52 (s, 1H), 3.13 (d, J=5.5 Hz, 2H), 1.55 (m, 7H). HRMS (ESI, m/z) Calculated for C₁₈H₁₈ClN₂O₄ [M−H]⁻: 361.0955, found: 361.0954.

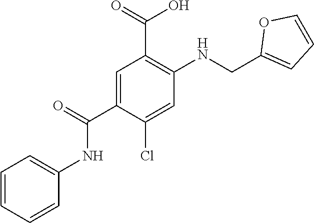 [SMILES: O=C(Nc1ccccc1)c1cc(C(=O)O)c(NCc2ccco2)cc1Cl]

### 4-Chloro-2((furan-2-ylmethyl)amino)-5-(phenylcarbamoyl)benzoic Acid (48g)

According to the General Procedure J, the title compound (153 mg, 40%) was obtained as a white solid.

¹H NMR (300 MHz, DMSO-d6 δ (ppm) 13.24 (s, 1H), 10.29 (s, 1H), 8.49 (s, 1H), 7.98 (s, 1H), 7.72-7.59 (m, 3H), 7.32 (t, J=7.8 Hz, 2H), 6.99 (s, 1H), 6.37 (s, 1H), 4.56 (d, J=5.5 Hz, 2H); HRMS (ESI, m/z) Calculated for C₁₉H₁₆ClN₂O₄ [M+H]⁺: 369.0642, found: 369.0656.

### Example 2: Aβ Oligomerization Studies

Aβ Oligomer Formation Assay with Single-Site biotin-Aβ(1-42)

Purpose:

The purpose of this assay was to determine the effect that compounds have on inhibiting the oligomerization of biotinylated amyloid-beta(1-42) (bioAbeta42).

As bioAbeta42 oligomerizes in the test plate, compounds with inhibitory activity will keep the bioAbeta42 in a monomeric state an, consequently, reduce the amount of oligomers. Once the oligomerization step is complete, the amount of oligomeric bioAbeta42 is determined using ELISA method with NeutrAvidin™-coated plate. NeutrAvidin™-bound oligomers are then detected by addition of Horseradish peroxidase (HRP) labeled streptavidin. Monomeric bioAbeta42 will not have extra biotin site, so that only oligomeric bioAbeta42 will be able to bind the streptavidin. HRP substrate is added to the wells and detected by absorbance in the plate reader giving an end point, total oligomer reading. A positive control (DMSO only) will give 100% oligomerization. Incubation with a negative control (0.1% Tween-20) will give 100% inhibition of oligomerization for a given run. Compounds, including a known standard compound (TRV1471, see U.S. Pat. No. 9,938,249), were analyzed to determine if they were effective at inhibiting oligomerization versus the control. The assay can detect oligomer species. This method was adapted from the method described in LeVine, Anal Biochem, 2006.

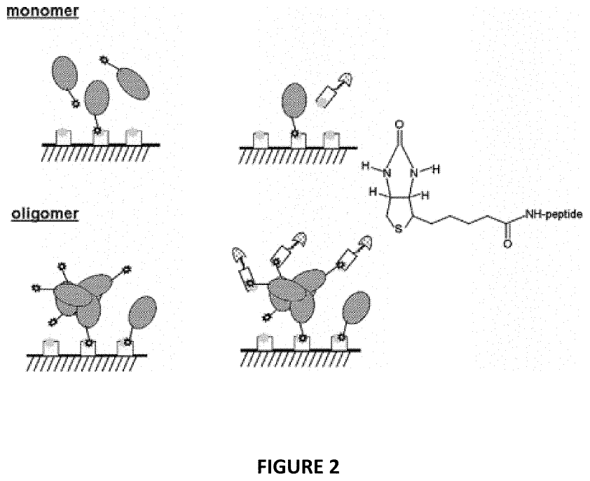

A schematic of this assay is provided in FIG. 2.

Procedure

A NeutrAvidin™-coated plate is prepared by coating an ELISA plate (Costar 9018) with 50 μl of 1 μg/ml NeutrAvidin™ (NA) in 10 mM sodium phosphate buffer, pH 7.5. NeutrAvidin™ can be prepared as 1 mg/ml (1000×) in DDW/10% glycerol and stored at −80° C. until use. The plate is sealed with adhesive film and stored at 4° C. overnight. The plate wells are then blocked for 2 hours at room temperature (RT) with 250 μl/well of OFB-T buffer. Plates may be unsealed during the 2 h blocking step.

The bio-Aβ(1-42) peptide is prepared by dissolving bio-Abeta peptide in HFIP at a concentration of 0.1 mg/ml. The peptide solution can be stored at −80° C. until use and thawed immediately before use.

For the assay 30 μl* bio-Abeta (for 6 plates, 20 cpds) is mixed with 50 μl of HFIP in a tube, vortexed, and then dried to a thin film under N₂ stream. (*Use 15 μl for 3 plates, 10 cpds) 50 μl of trifluoroacetic acid (TFA) is added to the tube, vortexed and incubated 10 min at RT in hood to disaggregate seeds, vortexed again, and then dried to a thin film under N₂ stream. 50 μl of HFIP is added, mixed, and dried under N₂ stream to remove residual TFA.

DMSO (1304 μl; 2×652 μl) is then added to the tube to provide a final peptide concentration of 2.3 μg/ml to make 50× bioAbeta42. Use within an hour or so. (* Use 652 μl for 3 plates, 10 cpds)

The assay is performed according to the following steps:
1. Prepare solutions of a test compound in a range of concentrations (e.g., 2 mM, 1 mM, 500 uM, 250 uM, 125 uM, 62.5 uM, and 31 uM of test compound) in 100% DMSO (Costar 3365).
2. Aliquot 3.4 μl of each concentration of the test compounds into the bottom of each well of a 96-well polypropylene plate.
3. Pipet 2 μl/well of the 50× bioAbeta42 peptide into the bottom of each well of three 96-well polypropylene plate (Costar 3365). Make sure every well has similar amount of bioAbeta drop.
4. Add 340 ul (2×170 ul) of Oligomer Formation Buffer to compounds:OFB-diluted test compounds. Precede to step 5 immediately.
5. Add 100 μl of OFB-diluted test compound to 50× bioAbeta42 and start oligomer r×n
6. Incubate plate for 1 hr at RT without shaking
7. Stop the reaction by addition of 50 μl of 0.3% Tween 20 in dH₂O

Oligomer formation is then measured as follows:
1. Let a NeutrAvidin™-coated plate come to room temperature.
2. Remove blocking solution from NeutrAvidin™-coated plate.
3. Add 50 μl of oligomer preparation from the wells in the assay plate to a corresponding well of the NeutrAvidin™-coated plate. Seal and incubate for 2 hr at RT with shaking.
4. Wash on plate washer (3×10 sec) with 250 μl/well with TBST (20 mM Tris-HCl, pH 7.5/150 mM NaCl/0.1% Tween 20).
5. Add 50 μl of Streptavidin-HRP (1:20,000) in OFBT, seal and incubate for 1 h with shaking at 150 rpm.
6. Wash as in Step 3 and add 100 μl of TMB/H₂O₂ substrate solution to each well. Stop reaction after 5-10 min with 100 μl of 2% v/v sulfuric acid.
7. Read the absorbance at 450 nm in a plate reader.

Buffers

TBST [20 mM Tris-HCl, pH 7.5-150 mM NaCl—0.1% Tween 20]

| | | |
|---|---|---|
| 1M Tris-HCl, pH 7.5 | 80 | ml |
| NaCl | 33.88 | g |
| Tween 20 (Research Organics) | 4 | ml |

| | |
|---|---|
| Distilled H₂O | 4 liters |

Oligomer Formation Buffer [20 mM sodium phosphate—150 mM NaCl, pH 7.5]

| | |
|---|---|
| Na₂HPO₄•7H₂O | 4.66 g |
| NaH₂PO₄•H₂O | 0.36 g |
| NaCl | 8.47 g |
| Distilled H₂O | 1 liter |

Blocking buffer (OFB-T buffer): Oligomer Formation Buffer+Tween 20 [20 mM sodium phosphate—150 mM NaCl, pH 7.5, 0.1% v/v Tween 20]

| Na₂HPO₄•7H₂O | 4.66 | g |
|---|---|---|
| NaH₂PO₄•H₂O | 0.36 | g |
| NaCl | 8.47 | g |
| Distilled H₂O | 1 liter | |
| Tween 20 | 1 | ml |

TMB/H₂O₂ Substrate Solution

Leave the reagents at RT for a while to warm up to RT. Add 4 μl of 35% H₂O₂ to 20 ml of Citrate/Acetate buffer and then add 200 μl of 10 mg/ml TMB solution to 20 ml of buffer.

Results

The results of testing exemplary compounds of the present application are provided in the table below.

| Compound No. | Anti-Aβ oligomerization IC50 (μM) |
|---|---|
| WW032 | 55.1 |
| WW048 | 135 |
| WW049 | 38.5 |
| WW052 | 21.7 |
| WW053 | 38.5 |
| WW064 | 46.1 |
| WW103 | 6.5 |

These results demonstrate the ability of compounds of the present application to act as anti-Aβ aggregation agents by inhibiting Aβ oligomerization.

### Example 3: Aβ Fibrillization Studies

Aβ ThT Aggregation Assay

Purpose

The purpose of this assay is to determine the effect that compounds have on inhibiting the aggregation of amyloid-beta. As amyloid-beta aggregates in the plate reader, it binds thioflavin T (ThT) and fluoresces. The fluorescence value is measured in the plate reader, giving a kinetic aggregation curve over time. A control sample (lacking compound) will give a 100% aggregation for a given run. Compounds then added to subsequent rows will show if they are effective at inhibiting aggregation versus the control. This is a 72 hour kinetic assay.

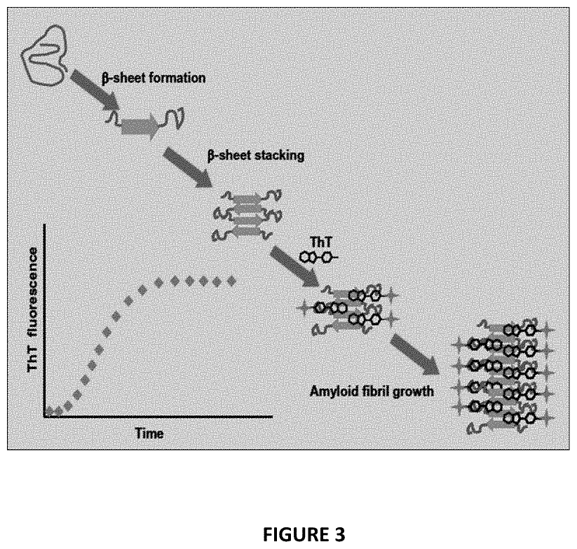

A schematic overview of this assay is provided in FIG. 3.

Procedure:
1. In the vial provided by Anaspec, dissolve the 1 mg of synthetic Aβ 1-40 in 1 mL HFIP. Vortex (10 s) and then sonicate it for approximately 5 min or until it becomes clear.
2. Remove the HFIP using a stream of argon in the chemistry fume hood. Make sure the sample is dry and only a waxy film remains.
3. Dissolve the waxy film in 1 mL of 20 mM tris base pH 10 (approx 230 μM Aβ), vortex and sonicate for approximately 5-10 minutes again until it becomes clear (might not always go clear—typically slightly cloudy).
4. Transfer this volume from the provided vial to a 15 mL glass vial.
5. Dilute the sample to 5.7 mL with tris base (gives 40 μM Aβ), which is additional 4.7 mL of tris base.
6. Drop the pH of the sample to 7.4 by adding small amounts of concentrated HCl, being sure not to overshoot. This takes approximately 10 μL. If pH is dropped too far use NaOH to bring it back up to 7.4.
7. Once the pH is at 7.4, filter the sample using a 0.2 μm syringe filter with 21 gauge needle.
8. Dilute the Aβ 1:1 (5.7 mL) with Tris HCl ThT Buffer (20 mM Tris, 8 uM ThT, 0.3M NaCl, pH 7.4) to give a total volume of 11.4 mL. This yields a final concentration of 20 μM Aβ.
9. This is then plated at 200 μL per well on a 96 well black plate, typically avoiding the outermost layer of wells as these tend to experience noticeable evaporation over the 3 day experiment.
10. Cover plate with clear plastic lid, put in plate reader and start appropriate pre-defined method (typically Kinetic ThT, version 4).

Buffers

| 20 mM Tris Base pH 10 | 20 mM Tris HCl ThT Buffer |
|---|---|
| MW = 121 g/mol | Same as tris base, plus |
| V(H₂O) = 400 mL | 0.3M NaCl (7.01 g in 400 mL) |
| m = 0.969 g | Drop pH to 7.4 with conc. HCl |
| | 8 mM ThT = Dissolve 5.1 mg in 2 mL tris |
| base. | Then dilute this 1000 times with tris |
| base. | |

Results

Compounds of the present application tested using the above assay were found to inhibit fibrillization of A3 oligomers. The results of testing exemplary compounds of the present application are provided in the table below.

| Compound No. | % inhibition of Aβ fibrillization (at 100 μ M) |
|---|---|
| WW016 | 81.32 |
| WW017 | 33.03 |
| WW064 | 39.24 |
| WW103 | 25.17 |

### Example 4: Anti-Neuroinflammation Studies

Neurotoxicity Assay

This study used differentiated (using phorbol myristate acetate, PMA) human acute monocytic leukemia cells, THP-1, cultured in RPMI 1640 medium supplemented with additives. Earlier studies with THP-1 cell line indicate that upon LPS/IFNγ activation, these cells release neurotoxic factors [Cheung Y T, et al. Neurotoxicology. 2009; 30(1):127-35]. These are remarkably plastic cells, which can potentially polarize to multiple cell lineages on the application of specific stimuli. On treatment with PMA, the suspension (floating cells) mature to a highly adherent phenotype with increased phagocytic ability (macrophage/microglia like). Typically, after PMA differentiation THP-1 monocytes, display increased cell surface expression of CD11b and CD14. PMA differentiated macrophage/microglia with a combination of LPS/IFNγ/amyloid beta peptide were used to activate the cells; and small synthetic molecules were tested. The cell-free supernatant generated from microglia were then transferred to previously mature neuronal cells lines.

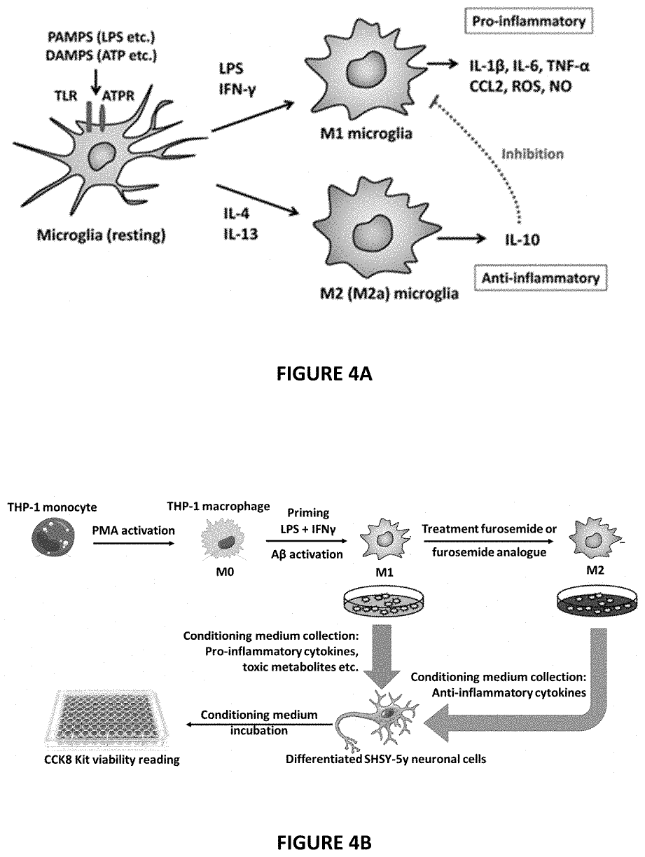

FIG. 4A shows the role of microglia involved in neuroinflammation and FIG. 4B schematically depicts the neurotoxicity (Ntox) assay, which is described in detail below.

Seeding, Activation, Priming, and Treatment of THP-1 Cells for the Ntox Assay

Day 1: Seeding

Seed (0.5×10⁶) THP-1 cells in each well of a 12-well tissue culture plate in warm complete growth medium up to 500 μl to 1 ml per well. The composition of the complete growth medium includes the following additives to 50 ml of RPMI 1640:
Tetracycline free heat-inactivated FBS 5 ml (Final concentration 10%)
Sodium pyruvate 500 μl (Final concentration one mM)
beta-mercaptoethanol five μl of 0.5 M prepared from a 14.3 M stock in RPMI 1640 (Final concentration 50 μM)
Antibiotic-Antimycotic (Anti-Anti) 500 μl of 100× (10,000 units penicillin, 10 mg streptomycin, and 25 μg amphotericin B per ml).

Use two T75 flasks with cells growing for at least three days in complete growth medium.

Day 2: Differentiation

Collect suspension cells from each well and centrifuge at 1000 r.p.m in a swinging bucket centrifuge for 5 minutes and suspend (500 μl) in warm Differentiating medium RPMI 1640 with the following additives (50 ml):
Tetracycline free heat-inactivated FBS 3.25 ml (Final concentration 6.25%)
Sodium pyruvate 500 μl (Final concentration one mM)
PMA 50 μl (Final concentration 150 nM) 1000× aliquots prepared in the −20 freezer

Day 3: Activation and Treatment

Warm RPMI 1640 medium and prepare Experimental medium RPMI 1640 with the following additives (50 ml):
Tetracycline free heat-inactivated FBS 2.5 ml (Final concentration 5%)
Sodium pyruvate 500 μl (Final concentration one mM)

The THP-1 monocytes should have adhered to the plate by now; these are defined as M0 macrophages. Remove the differentiating medium containing PMA and add the Experimental medium with stimulants at a final concentration of IFNγ 400 ng/ml and LPS 1 μg/ml. These stimulants are the first stimulus for the conversion into M1 macrophages. Keep the first stimulus on for three hours and add 25 μM of compounds in the required wells (This is aimed at converting M1→M2 macrophages/microglia like cells).

Resuspend 12 μl of 0.5 mM of Aβ₁₋₄₂ in 1200 μl of DPBS without calcium and magnesium/sterile filtered oligomer forming buffer pH 7.2-7.4 vortex at 5 r.p.m. on the bench top vortex for 30 seconds. Place this on the shaker at 300 r.p.m. for 1.5 hours. After four hours of addition of LPS and IFNγ, most THP-1 monocytes adhere to the surface of the plate, now add 50 μl of resuspended Aβ to each well of THP-1 cells. It is important to add the peptide drop-by-drop evenly distributing it in each well of two 12 well plates containing 500 μl of media at the final concentration 500 nM Aβ₁₋₄₂. Place the cells in the culture incubator for 48 hours before extracting the cells/supernatant for further processing.

Day 5: Collection

Collect the supernatant from each well place it in a labeled 1.5 ml centrifuge tube, centrifuge at 3000 r.p.m. The collected supernatant may be used for end-point assays like the neuron cell viability, or determination of cytokine levels. Wash the adherent macrophages with ice-cold PBS and collecting any floating cells by centrifugation at 3000 r.p.m. Use the adhered cells lysates for immunoblotting with anti-pro-IL-1β (34 kDa) and anti-Tex264 antibodies. The intact cells may be used for functional assays like the migration assay, phagocytosis assay, determination of cell surface markers indicating M0, M1, M2 phenotypes, detection of reactive oxygen species and GSH (markers for oxidative stress), caspases and NLRP3 inflammasome markers.

Maturation of SH-SY5Y (ATCC: CRL-2266)

Day 1: Seed cells at a density of 5000 cells/well of 96 well plate in growth medium (Growth medium DMEM/F12 with 5% FBS and antibiotics).

Day 2: Replace growth medium with 2.5% FBS containing DMEM/F12 supplemented with Retinoic acid at a final concentration of 10 μM. Keep for 7-10 days replacing differentiating medium every other day. Mature cells express higher levels of NMDA receptors. Apply microglial supernatant on the mature-differentiated SH-SY5y cells, measure the neuronal viability, cell cycle analysis for apoptosis and other cell death markers.

Results

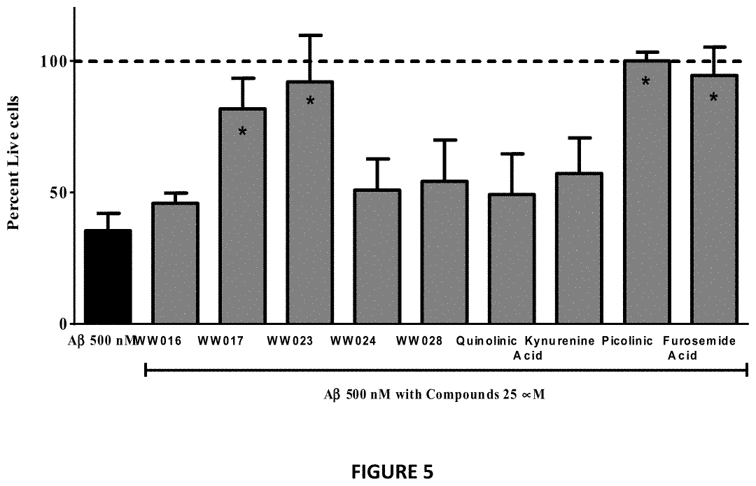

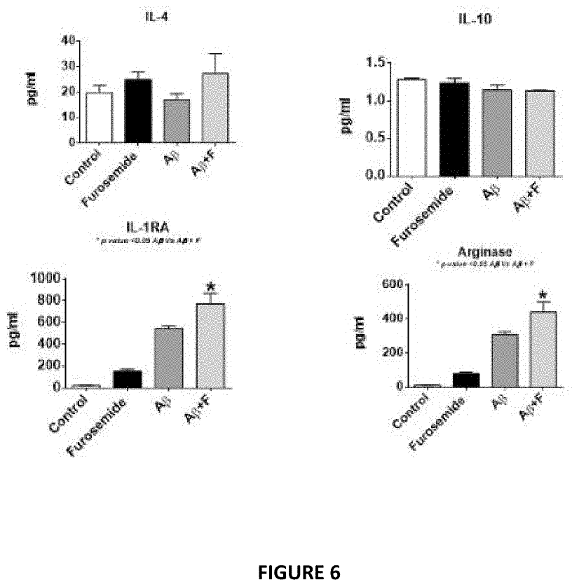

The results of these studies are shown in FIGS. 5 and 6. As shown in FIG. 5, compounds of this application are effective in rescuing neuronal cells under Aβ induced neuroinflammation, as indicated by the increase in percent live cells in comparison to the control with no compound added. Also, as shown in FIG. 6, the addition of furosemide increases the concentrations of anti-inflammatory cytokines. Furosemide also inhibits the production and release of cytokines including IL-6, IL-8, and TNF-α from peripheral mononuclear cells (data not shown).

### Example 5: Activity Studies

A series of furosemide analogues as defined herein were studied for activity as inhibitors of Aβ-misfolding and for activity as inhibitors of neuroinflammation.

Methods

The assay methods used in the present example are similar to those used in previous examples, with minor modifications.

β-Amyloid Oligomerization Assay

This assay was adapted from LeVine (2006). Biotinylated Aβ₁₋₄₂ was purchased from AnaSpec (Freemont, Calif., United States). An ELISA plate (Costar 9018) was coated with 50.0 μL of a stock solution containing 1.00 μg/mL NeutrAvidin in sodium phosphate buffer (10.0 mM, pH 7.50). The plate was sealed and stored at 4° C. overnight prior to blocking for 2 h at room temperature with 200 μL/well of OFB-T buffer [20.0 mM sodium phosphate, 150 mM NaCl, pH 7.50, 0.100% (v/v) Tween 20]. Then, 20.0 μL of Aβ₁₋₄₂ stock solution (0.100 mg/mL) was treated with HFIP and dried under a stream of argon. One hundred microliter of trifluoroacetic acid (TFA) was added to the tube and the sample was dissolved using a vortex mixer prior to drying under a stream of argon. HFIP was added and dried under a stream or argon to remove residual TFA. The biotinylated Aβ₁₋₄₂ was then dissolved in 870 μL of DMSO, and 2.00 μL of the solution was added to each well of a 96-well polypropylene plate (Costar 3365) followed by 100 μL of test compound diluted in OFB-T buffer (various concentrations). The plate was incubated for 1 h at room temperature without shaking, and then stopped by the addition of 50.0 μL of 0.300% (v/v) Tween 20 in MilliQ® water. 50.0 μL of the biotinylated A0142/compound solution was added to each well of the NeutrAvidin™-coated plate (after removing blocking solution) and the plate was sealed and incubated for 2 h with shaking at 150 rpm. The plate was washed three times with TBST solution [20.0 mM Tris-HCl, 34.0 mM sodium chloride, pH 7.50, 0.100% (v/v) Tween 20], then 50.0 μL of Streptavidin-HRP (1:20,000) in OFB-T buffer was added and the plate was sealed and incubated for 1 h with shaking at 150 rpm. The plate was again washed three times with TBST, followed by addition of 100 μL of tetramethylbenzidine/H₂O₂ substrate solution to each well. The reaction was stopped after 10-30 min by the addition of 100 μL of 2.00% (v/v) aqueous sulfuric acid prior to reading absorbance at 450 nm in a plate reader.

β-amyloid ThT Aggregation Assay

This assay was adapted from LeVine (1993). Aβ₁₋₄₀ (>95%) was purchased from AnaSpec (Freemont, Calif., United States) and stored at −80° C. All other reagents were of the highest available purity, purchased from Sigma-Aldrich (Oakville, ON, Canada), and used without further purification. All water used in the assays was micropore filtered and deionized (MilliQR). Aβ₁₋₄₀ (1.00 mg) was dissolved in hexafluoro-2 propanol (HFIP) and sonicated for 30 min to disassemble any pre-formed aggregates. HFIP was removed using a stream of argon gas prior to dissolution of Aβ₁₋₄₀ in 1.00 mL Tris base (20.0 mM, pH 10.0) using vortex and 10 min sonication. The solution was then further diluted with 4.70 mL of Tris base followed by adjusting to pH 7.40 using concentrated hydrochloric acid and then filtered using a 0.200 mm syringe filter. The pretreated Aβ₁₋₄₀ was diluted with an equal volume (5.70 mL) of 8.00 mM

ThT in Tris (20.0 mM, pH 7.40, 300 mM NaCl) and 200 mL aliquots of this solution [20.0 mM Aβ₁₋₄₀ and 4.00 mM ThT in Tris (20.0 mM, pH 7.40, 150 mM NaCl)] were added to wells of a black polystyrene 96-well plate. 4.00 mL of test compound solutions at various concentrations were added to each well. Each sample was performed in triplicate and MilliQR water alone served as a vehicle control. Plates were sealed and incubated in a microplate reader (Tecan Genios) at 37.0° C. with fluorescence measurements recorded (lex=450 nm, lem=480 nm) every 15 min after first being shaken at high intensity for 15 s and then allowed to settle for 10 s before each reading.

Assay for Testing Anti-Inflammation Activity Using SIM-A9 Cell Line

SIM-A9 cells are maintained in Dulbecco's modified eagle medium: nutrient mixture F-12 (DMEM-F12) with 10% fetal bovine serum, 5% horse-serum and antibiotic-antimycotic (Anti-anti). To passage the cells, transfer all medium and floating cells from flask to a centrifuge tube. The adherent cell layer is washed with Ca++/Mg++ free Dulbecco's phosphate-buffered saline (D-PBS). Collect rinse solution to the same centrifuge tube. Cells are detached by 1 mM EDTA, 1 mM EGTA and 1 mg/mL glucose solution. Observe cells under a microscope until cell layer is dispersed. All collected cells are centrifuged at 125×g for 5 to 10 minutes. Discard supernatant and resuspend the cell pellet in growth medium.

SIM-A9 cells were seeded 24 hr before experiment (˜90% confluency before activation). Culturing medium was replaced with reduced FBS DMEM-F12 medium (containing 5% FBS+2.5% horse serum) with required LPS concentration (final volume is 1 mL/well). The conditioning medium and lysate were harvested for cytokine and cell marker detection.

Enzyme-Inked Immunosorbent Assay (ELISA)

The cytokines secreted from SIM-A9 were quantified using ELISA kits following manufacture's instructions. Limits of detection for the ELISA kits were IL-6 (2.5-500 μg/mL) and TNF-α (5-1000 μg/mL). Briefly, the high-binding plates were coated with 100 μL/well with diluted capture anti-bodies (1:250) for overnight at 4° C. The coated plates were blocked with the diluent for 1 hour before the assay. Each sample was diluted accordingly and added to the plates for 2-hour incubation at room temperature. Plates were then washed by 250 μL/well PBS with 0.05% Tween-20 and incubated with detection anti-bodies (1:250 in assay diluent) for 1 hour at room temperature. After another washing step, 1:250 diluted avidin-HRP (horseradish peroxidase) was added and incubated for 30 mins. 100 μL TMB-substrate was added after the final washing and the plate was incubated in dark until the signal was sufficiently developed. The reaction was stop by 50 μL 2 N sulfuric acid. The absorbance was measured at 450 nm with a correction of 570 nm using plate reader.

Western Blotting

After 24 hr activation by LPS, cell grown in 6-well plates were washed twice with ice-cold PBS and harvested in RIPA buffer supplemented with protease inhibitor cocktail. The whole-cell extracts were then centrifuged at 1,5000 rpm for 20 mins at 4° C. in order to remove cell debris. Protein concentrations were quantified using Micro BCA protein assay kit. The absorbance was measured at 595 nm using microplate reader.

Equal amounts of cellular protein were separated by sodium dodecyl sulfate-polyacrylamide gel electrophoresis (SDS-PAGE) and transferred onto polyvinylidene difluoride (PVDF) membranes at 100 V for 90 mins. The membranes were blocked for 1 hour in Tris-buffered saline (TBS), pH 7.4, with 0.1% Tween-20 (TBS-T) containing 10% skim milk. The membrane blot was then incubated overnight at 4° C. with primary antibodies against iNOS (1:1000), COX-2 (1:1000) and Tubulin (1:5000) in TBS-T containing 5% skim milk. The membrane was washed with TBS-T 3×10 mins and incubated with goat anti-rabbit IgG-horseradish peroxidase (1:5,000) for 1 hour. After the washing step, the immunoblotting was visualized by chemiluminescence HRP-substrate.

Results

The results of the anti-Aβ oligomerization and inhibition of Aβ fibrillization studies are provided in the tables below.

TABLE
Synthetic molecules and their activity towards anti-Aβ oligomerization and fibrillization.
Compounds of Formula I

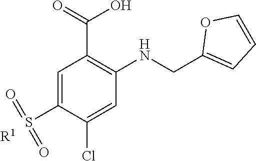

| | | | | Inhibition of Aβ | |
|---|---|---|---|---|---|
| | | Inhibition of Aβ | | fibrilllization at | |
| | | oligomerization, | | concentration 100 μM | |
| Compound | | μM | | Inhibition | |
| No. | R¹ | IC₅₀ | R² | % | Statistic^{b} |
| Furosemide | NH₂ | ND | N/A | 8.56 | NS |
| SI-W030 | 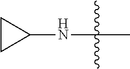 [SMILES: *NC1CC1] | ND | N/A | — | — |
| SI-W032 | 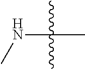 [SMILES: *NC] | 53.01 ± 3.37 | 0.886 | Pro- fibrilliza- tion | — |
| SI-W035 | 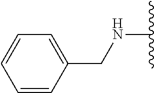 [SMILES: *NCc1ccccc1] | ND | N/A | — | — |
| SI-W036 | 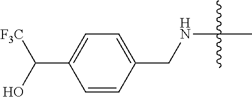 [SMILES: *NCc1ccc(C(O)C(F)(F)F)cc1] | 68.5 ± 18.75 | 0.927 | Pro- fibrilliza- tion | — |
| SI-W049 | 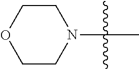 [SMILES: *N1CCOCC1] | 41.16 ± 12.78 | 0.87 | Pro- fibrilliza- tion | — |
| SI-W052 | 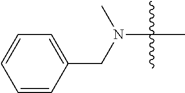 [SMILES: *N(C)Cc1ccccc1] | 21.92 ± 7.36 | 0.905 | — | — |
| SI-W053 | 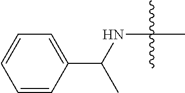 [SMILES: *NC(C)c1ccccc1] | 41.15 ± 20.41 | 0.883 | — | — |
| SI-W056 | 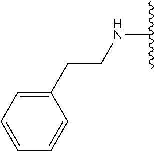 [SMILES: *NCCc1ccccc1] | ND | N/A | — | — |
| SI-W057 | 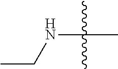 [SMILES: *NCC] | 137.39 ± 11.88 | 0.958 | — | — |
| SI-W062 | 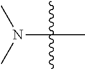 [SMILES: *N(C)C] | 113.13 ± 22.83 | 0.88 | Pro- fibrilliza- tion | — |
| SI-W063 | 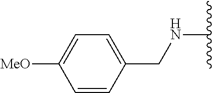 [SMILES: *NCc1ccc(OC)cc1] | ND | N/A | — | — |
| SI-W064 | 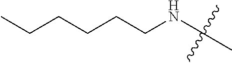 [SMILES: *NCCCCCC] | 47.81 ± 14.35 | 0.838 | 39.24 | **** |
| SI-W065 | 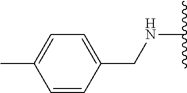 [SMILES: *NCc1ccc(C)cc1] | ND | N/A | — | — |
| SI-W080 | 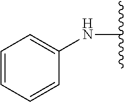 [SMILES: *Nc1ccccc1] | 167.53 ± 28.97 | 0.907 | 17.42 | * |
| SI-W082 | 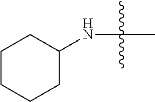 [SMILES: *NC1CCCCC1] | 115.52 ± 13.82 | 0.887 | — | — |
| SI-W096 | 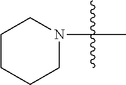 [SMILES: *N1CCCCC1] | ND | N/A | 19.08 | * |

Compounds of Formula IV

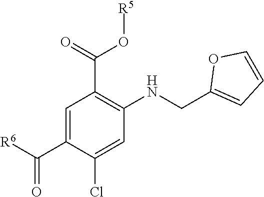

| | | | | | Inhibition of Aβ | |
|---|---|---|---|---|---|---|
| | | | | | fibrillization at | |
| | | | Inhibition of Aβ | | concentration 100 μM | |
| | | | oligomerization, μM | | Inhibition | |
| Compound No. | R⁶ | R⁵ | IC₅₀ | R² | % | Statistic |
| SIV-W163 | NH₂ | Me | | | | |
| SIV-W164 | NH₂ | H | ND | N/A | — | — |
| SIV-W155 | 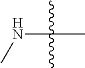 [SMILES: *NC] | Me | | | | |
| SIV-W168 | 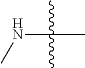 [SMILES: *NC] | H | ND | N/A | — | — |
| SIV-W172 | 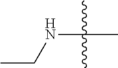 [SMILES: *NCC] | Me | | | | |
| SIV-W174 | 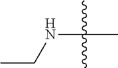 [SMILES: *NCC] | H | ND | N/A | — | — |
| SIV-W171 | 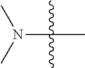 [SMILES: *N(C)C] | Me | | | | |
| SIV-W173 | 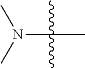 [SMILES: *N(C)C] | H | ND | N/A | — | — |
| SIV-W159 | 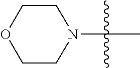 [SMILES: *N1CCOCC1] | Me | | | | |
| SIV-W166 | 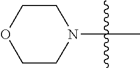 [SMILES: *N1CCOCC1] | H | ND | N/A | — | — |
| SIV-W160 | 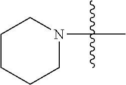 [SMILES: *N1CCCCC1] | Me | | | | |
| SIV-W167 | 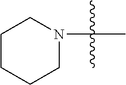 [SMILES: *N1CCCCC1] | H | ND | N/A | — | — |
| SIV-W178 | 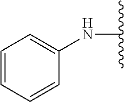 [SMILES: *Nc1ccccc1] | Me | | | | |
| SIV-W179 | 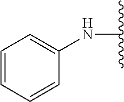 [SMILES: *Nc1ccccc1] | H | ND | N/A | — | — |

Compounds of Formula II

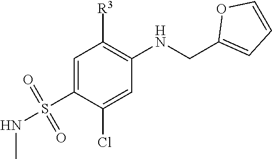

| | | | | Inhibition of Aβ | |
|---|---|---|---|---|---|
| | | | | fibrillization at | |
| | | Inhibition of Aβ oligomerization, | | concentration 100 μM | |
| | | μM | | Inhibition | |
| Compound No. | R³ | IC₅₀ | R² | % | Statistic |
| SII-W094 | 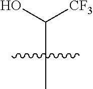 [SMILES: *C(O)C(F)(F)F] | | | — | — |
| SII-W100 | 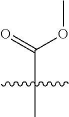 [SMILES: *C(=O)OC] | 55.95 ± 26.58 | 0.834 | 27.14 | *** |
| SII-W101 | 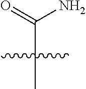 [SMILES: *C(N)=O] | 82.41 ± 28.54 | 0.843 | Pro- fibrillization | — |
| SII-W102 | 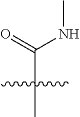 [SMILES: *C(=O)NC] | 102.59 ± 22.05 | 0.958 | Pro- fibrillization | — |
| SII-W103 | 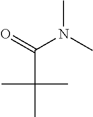 | 6.56 ± 2.47 | 0.947 | 25.17 | ** |
| SII-W109 | 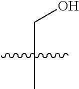 [SMILES: *CO] | 117.49 ± 3.95 | 0.971 | — | — |
| SII-W121 | 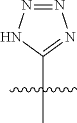 [SMILES: *c1nnn[nH]1] | 70.48 ± 15.7 | 0.927 | — | — |
| SII-W187 | 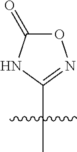 [SMILES: *c1noc(=O)[nH]1] | ND | N/A | — | — |

Compounds of Formula IIIb

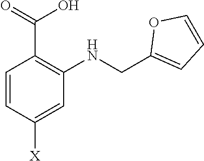

| | | Inhibition of Aβ | | Inhibition of Aβ fibrillization | |
|---|---|---|---|---|---|
| Compound | | oligomerization, μM | | at concentration 100 μM | |
| No. | X | IC₅₀ | R² | Inhibition % | Statistic |
| SIIIb-W001 | Cl | ND | N/A | 20.18 | * |
| SIIIb-W071 | F | 129.54 ± 25.82 | 0.958 | — | — |
| SIIIb-W073 | Br | 154.42 ± 33.79 | 0.846 | — | — |

Compounds of Formula IIIa

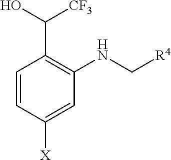

| | | | Inhibition of Aβ | | Inhibition of Aβ fibrillization | |
|---|---|---|---|---|---|---|
| Compound | | | oligomerization, μM | | at concentration 100 μM | |
| No. | X | R⁴ | IC₅₀ | R² | Inhibition % | Statistic |
| SIIIa-W013 | Cl | 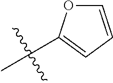 [SMILES: *c1ccco1] | ND | N/A | 60.72 | **** |
| SIIIa-W021 | Cl | 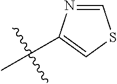 [SMILES: *c1cscn1] | ND | N/A | 27.88 | *** |
| SIIIa-W011 | F | 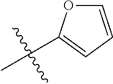 [SMILES: *c1ccco1] | 153.05 ± 48.01 | 0.840 | 5.14 | NS |
| SIIIa-W016 | Br | 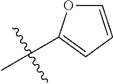 [SMILES: *c1ccco1] | ND | N/A | 81.32 | **** |
| SIIIa-W017 | Br | 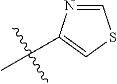 [SMILES: *c1cscn1] | ND | N/A | 33.03 | **** |
| SIIIa-W003 | OMe | 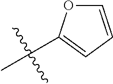 [SMILES: *c1ccco1] | ND | N/A | 36.80 | **** |
| SIIIa-W005 | OMe | 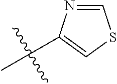 [SMILES: *c1cscn1] | 129.69 ± 28.37 | 0.865 | 6.49 | NS |

‘ND’ denotes the inhibition activity was not detected over 200 μM
‘S#” denotes a structure of Formula #
^{a}Compound 100 forms fibrillization in assay buffer at 200 μM
^{b}The statistical analysis was performed using one-student test comparing to positive control DMSO.
ns: not significant, p > 0.05; *: p ≤ 0.05; **: p ≤ 0.01; ***: p ≤ 0.001; ****: p ≤ 0.0001

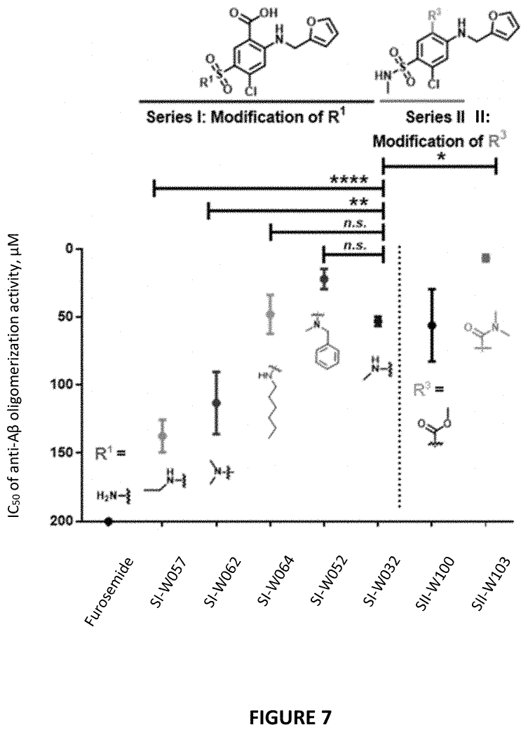

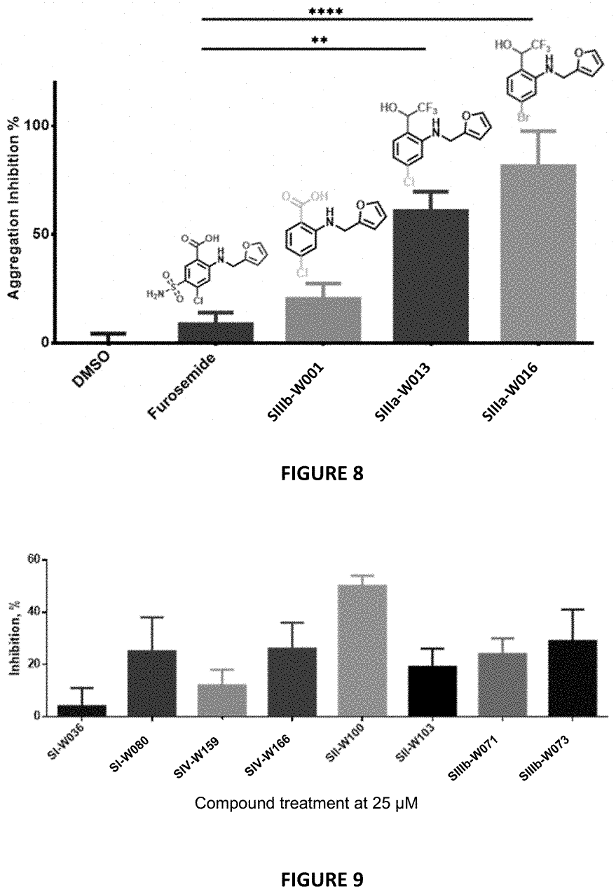

FIG. 7 illustrates how changing functional groups in Regions A and B can be used to alter anti-Aβ oligomerization activity of the compound of Formula Z. FIG. 8 illustrates how changing functional groups in Region B, as well as the nature of the halide, can be used to alter anti-Aβ fibrillization activity. Of the compounds studied in this Example, SII-W103 exhibited the lowest IC₅₀, of 6.5 μM, towards anti-Aβ oligomerization, and SIIIa-W016 exhibited the greatest inhibition, 81%, towards anti-Aβ fibrillization.

The results of the anti-inflammation studies are provided in the following tables.

| 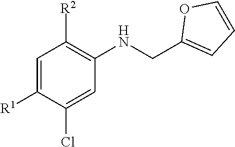 | | | |
|---|---|---|---|
| | | | TNF-α % |
| R¹ | R² | BBB score | Inhibition |
| SO₂NH₂ | COOH | 3.29 | 31.0 |
| SO₂NH₂ | H | 4.12 | Not active |
| SO₂NH₂ | 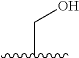 [SMILES: *CO] | 4.22 | Not active |
| SO₂NH₂ | 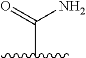 [SMILES: *C(N)=O] | 3.93 | Not active* |
| SO₂NH₂ | 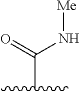 [SMILES: *C(=O)NC] | 4.14 | 38.0 |
| SO₂NH₂ | 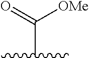 [SMILES: *C(=O)OC] | 4.41 | 50.0 |
| SO₂NH₂ | 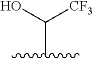 [SMILES: *C(O)C(F)(F)F] | 3.55 | Not active |
| H | 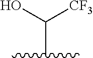 [SMILES: *C(O)C(F)(F)F] | 4.83 | Not active* |

| R¹ | R² | BBB score |
|---|---|---|
| SO₂NH₂ | 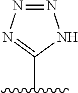 [SMILES: *c1nnn[nH]1] | 3.99 |
| H | 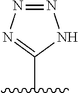 [SMILES: *c1nnn[nH]1] | 4.84 |
| SO₂NH₂ | 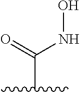 [SMILES: *C(=O)NO] | 3.45 |
| H | 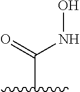 [SMILES: *C(=O)NO] | 4.68 |
| H | 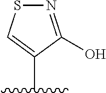 [SMILES: *c1csnc1O] | 4.75 |
| H | 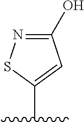 [SMILES: *c1cc(O)ns1] | 4.75 |

Compounds were tested at 25 μM on SIM-A9 cells.
BBB = blood-brain barrier
core (see Gupta et al, ACS Chemical Neuroscience 2020, 11, 2, 205-224).
*Indicates that compounds were active at lower concentrations.

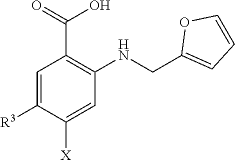

| | | TNF-α % |
|---|---|---|
| R³ | BBB score | Inhibition |
| SO₂NH₂ | 3.29 | 31 |
| H | 3.78 | 18 |
| 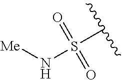 [SMILES: *S(=O)(=O)NC] | 4.31 | 19.2 |
| 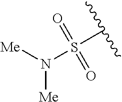 [SMILES: *S(=O)(=O)N(C)C] | 3.96 | 9.2 |
| 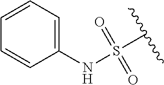 [SMILES: *S(=O)(=O)Nc1ccccc1] | 3.39 | 25 |
| 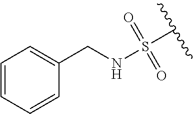 [SMILES: *S(=O)(=O)NCc1ccccc1] | 3.87 | 32 |
| 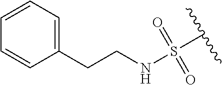 [SMILES: *S(=O)(=O)NCCc1ccccc1] | 3.82 | 27 |
| 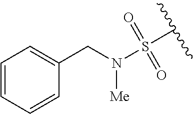 [SMILES: *S(=O)(=O)N(C)Cc1ccccc1] | 3.41 | Not active |
| 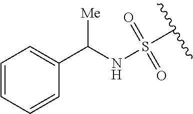 [SMILES: *S(=O)(=O)NC(C)c1ccccc1] | 3.82 | Not active |
| 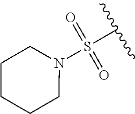 [SMILES: *S(=O)(=O)N1CCCCC1] | 3.86 | Not active |
| 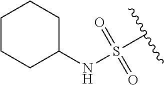 [SMILES: *S(=O)(=O)NC1CCCCC1] | 4.18 | 37.2 |
| 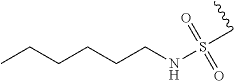 | 4.19 | Not active |

The results shown above indicate that the sulfonyl group is not necessary at Region A but can improve anti-inflammatory activity.

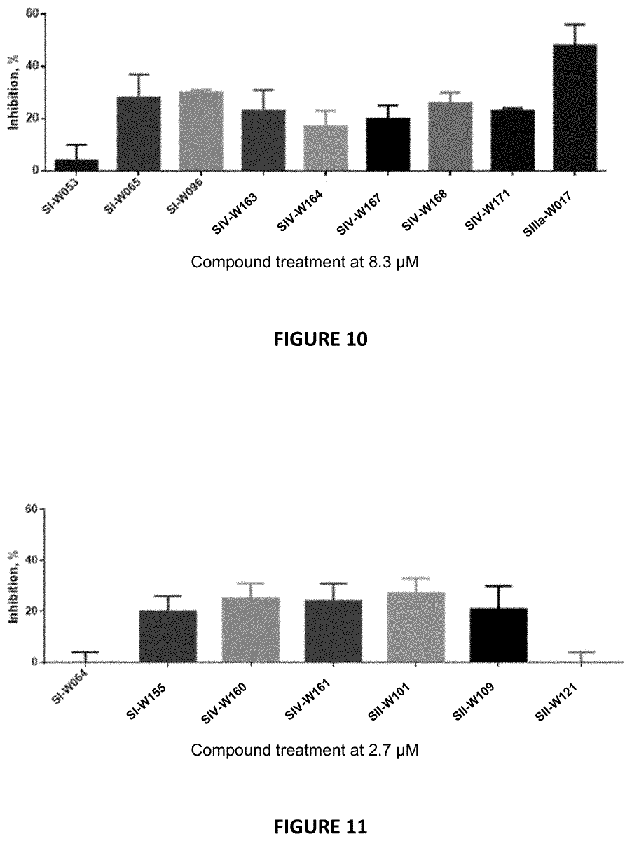

FIGS. 9-11 depict the results of compound activity on TNF-α production from 5 ng/mL LPS stimulated AIM-A9 cells, as an indication of anti-inflammatory activity.

The following table shows the results of compound activity on TNF-α production from LPS stimulated AIM-A9 cells using the compounds at a concentration of 2.7 μM.

| Compound | | TNF-α |
|---|---|---|
| identifier | Compound structure | Inhibition |
| WW-003 | 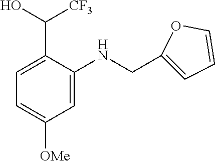 [SMILES: COc1ccc(C(O)C(F)(F)F)c(NCc2ccco2)c1] | 28% |
| | Chemical Formula: C₁₄H₁₄F₃NO₃ | |
| | Molecular Weight: 301.27 | |
| WW-005 | 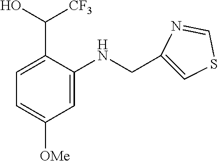 [SMILES: COc1ccc(C(O)C(F)(F)F)c(NCc2cscn2)c1] | 27% |
| | Chemical Formula: C13H13F3N2O2S | |
| | Molecular Weight: 318.31 | |
| WW-011 | 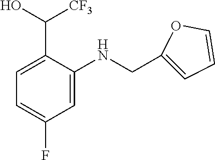 [SMILES: OC(c1ccc(F)cc1NCc1ccco1)C(F)(F)F] | 25.9% |
| | Chemical Formula: C13H11F4NO2 | |
| | Molecular Weight: 289.23 | |
| WW-013 | 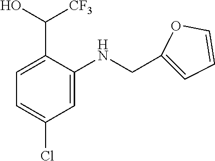 [SMILES: OC(c1ccc(Cl)cc1NCc1ccco1)C(F)(F)F] | 33% |
| | Chemical Formula: C13H11ClF3NO2 | |
| | Molecular Weight: 305.68 | |
| WW-016 | 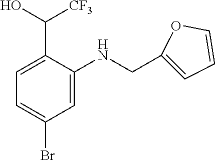 [SMILES: OC(c1ccc(Br)cc1NCc1ccco1)C(F)(F)F] | 34.8% |
| | Chemical Formula: C13H11BrF3NO2 | |
| | Molecular Weight: 350.14 | |
| WW-017 | 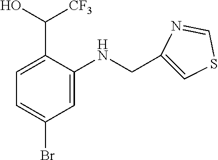 [SMILES: OC(c1ccc(Br)cc1NCc1cscn1)C(F)(F)F] | 31% |
| | Chemical Formula: C12H10BrF3N2OS | |
| | Molecular Weight: 367.18 | |
| WW-021 | 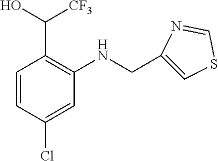 [SMILES: OC(c1ccc(Cl)cc1NCc1cscn1)C(F)(F)F] | 38% |
| | Chemical Formula: C12H10ClF3N2OS | |
| | Molecular Weight: 322.73 | |
| WW-024 | 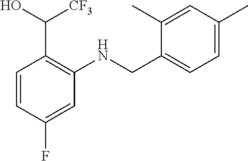 [SMILES: Cc1ccc(CNc2cc(F)ccc2C(O)C(F)(F)F)c(C)c1] | 11.2% |
| | Chemical Formula: C17H17F4NO | |
| | Molecular Weight: 327.32 | |
| WW-028 | 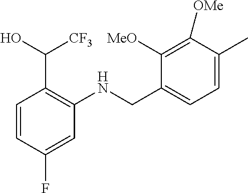 [SMILES: COc1c(C)ccc(CNc2cc(F)ccc2C(O)C(F)(F)F)c1OC] | 19.9% |
| | Chemical Formula: C17H17F4NO3 | |
| | Molecular Weight: 359.32 | |
| WW-052 | 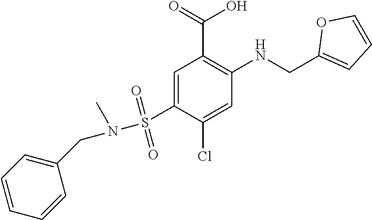 [SMILES: CN(Cc1ccccc1)S(=O)(=O)c1cc(C(=O)O)c(NCc2ccco2)cc1Cl] | 24.1% |
| | Chemical Formula: C₂₀H₁₉ClN₂O₅S | |
| | Molecular Weight: 434.89 | |
| WW-056 | 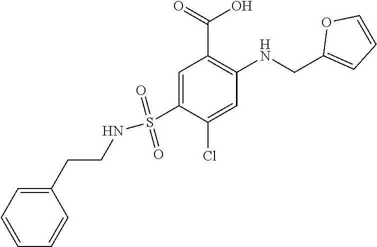 [SMILES: O=C(O)c1cc(S(=O)(=O)NCCc2ccccc2)c(Cl)cc1NCc1ccco1] | 13% |
| | Chemical Formula: C₂₀H₁₉ClN₂O₅S | |
| | Molecular Weight: 434.89 | |
| WW-065 | 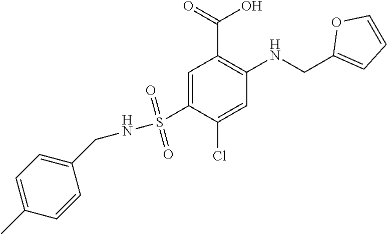 [SMILES: Cc1ccc(CNS(=O)(=O)c2cc(C(=O)O)c(NCc3ccco3)cc2Cl)cc1] | 20% |
| | Chemical Formula: C₂₀H₁₉ClN₂O₅S | |
| | Molecular Weight: 434.89 | |
| WW-073 | 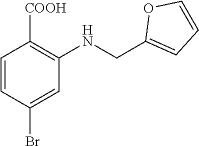 [SMILES: O=C(O)c1ccc(Br)cc1NCc1ccco1] | 27% |
| | Chemical Formula: C₁₂H₁₀BrNO₃ | |
| | Molecular Weight: 296.12 | |
| WW-082 | 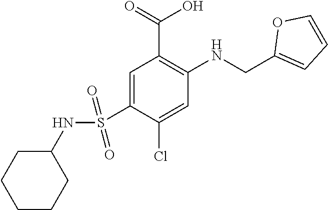 [SMILES: O=C(O)c1cc(S(=O)(=O)NC2CCCCC2)c(Cl)cc1NCc1ccco1] | 11% |
| | Chemical Formula: C₁₈H₂₁ClN₂O₅S | |
| | Molecular Weight: 412.89 | |
| WW-096 | 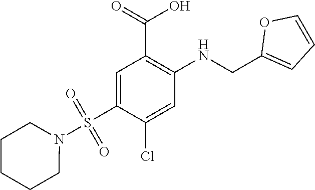 [SMILES: O=C(O)c1cc(S(=O)(=O)N2CCCCC2)c(Cl)cc1NCc1ccco1] | 11% |
| | Chemical Formula: C₁₇H₁₉ClN₂O₅S | |
| | Molecular Weight: 398.86 | |
| WW-100 | 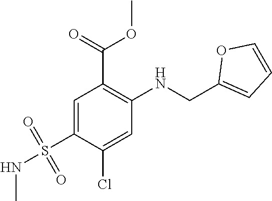 [SMILES: CNS(=O)(=O)c1cc(C(=O)OC)c(NCc2ccco2)cc1Cl] | 31% |
| | Chemical Formula: C₁₄H₁₅ClN₂O₅S | |
| | Molecular Weight: 358.79 | |
| WW-101 | 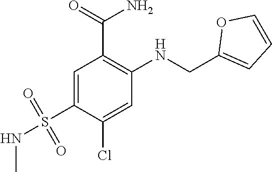 [SMILES: CNS(=O)(=O)c1cc(C(N)=O)c(NCc2ccco2)cc1Cl] | 27% |
| | Chemical Formula: C₁₃H₁₄ClN₃O₄S | |
| | Molecular Weight: 343.78 | |
| WW-102 | 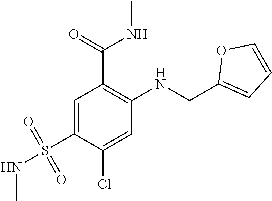 [SMILES: CNC(=O)c1cc(S(=O)(=O)NC)c(Cl)cc1NCc1ccco1] | 25% |
| | Chemical Formula: C₁₄H₁₆ClN₃O₄S | |
| | Molecular Weight: 357.81 | |
| WW-109 | 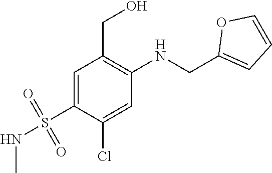 [SMILES: CNS(=O)(=O)c1cc(CO)c(NCc2ccco2)cc1Cl] | 21% |
| | Chemical Formula: C₁₃H₁₅ClN₂O₄S | |
| | Molecular Weight: 330.78 | |
| WW-121 | 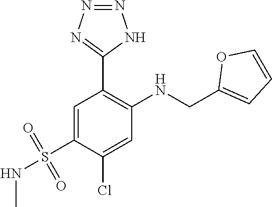 [SMILES: CNS(=O)(=O)c1cc(-c2nnn[nH]2)c(NCc2ccco2)cc1Cl] | 2% |
| | Chemical Formula: C₁₃H₁₃ClN₆O₃S | |
| | Molecular Weight: 368.80 | |
| WW-155 | 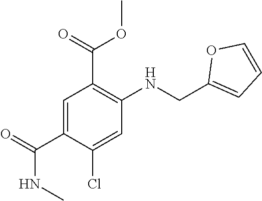 [SMILES: CNC(=O)c1cc(C(=O)OC)c(NCc2ccco2)cc1Cl] | 20% |
| | Chemical Formula: C₁₅H₁₅ClN₂O₄ | |
| | Molecular Weight: 322.75 | |
| WW-160 | 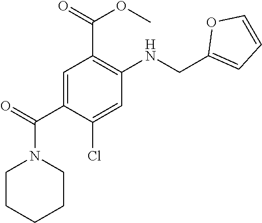 [SMILES: COC(=O)c1cc(C(=O)N2CCCCC2)c(Cl)cc1NCc1ccco1] | 25% |
| | Chemical Formula: C₁₉H₂₁ClN₂O₄ | |
| | Molecular Weight: 376.84 | |
| WW-161 | 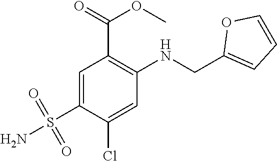 [SMILES: COC(=O)c1cc(S(N)(=O)=O)c(Cl)cc1NCc1ccco1] | 24% |
| | Chemical Formula: C₁₃H₁₃ClN₂O₅S | |
| | Molecular Weight: 344.77 | |
| WW-166 | 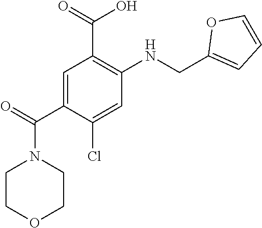 [SMILES: O=C(O)c1cc(C(=O)N2CCOCC2)c(Cl)cc1NCc1ccco1] | 25% |
| | Chemical Formula: C₁₇H₁₇ClN₂O₅ | |
| | Molecular Weight: 364.78 | |
| WW-168 | 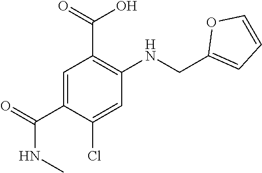 [SMILES: CNC(=O)c1cc(C(=O)O)c(NCc2ccco2)cc1Cl] | 27% |
| | Chemical Formula: C₁₄H₁₃ClN₂O₄ | |
| | Molecular Weight: 308.72 | |
| WW-172 | 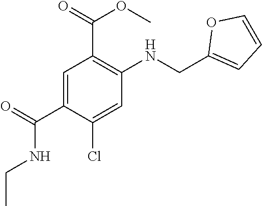 [SMILES: CCNC(=O)c1cc(C(=O)OC)c(NCc2ccco2)cc1Cl] | 18% |
| | Chemical Formula: C₁₆H₁₇ClN₂O₄ | |
| | Molecular Weight: 336.77 | |
| WW-173 | 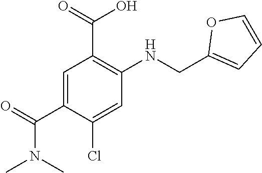 [SMILES: CN(C)C(=O)c1cc(C(=O)O)c(NCc2ccco2)cc1Cl] | 25% |
| | Chemical Formula: C₁₅H₁₅ClN₂O₄ | |
| | Molecular Weight: 322.75 | |
| WW-174 | 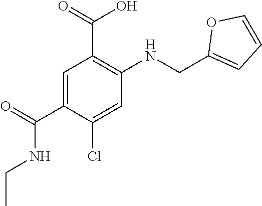 [SMILES: CCNC(=O)c1cc(C(=O)O)c(NCc2ccco2)cc1Cl] | 21% |
| | Chemical Formula: C₁₅H₁₅ClN₂O₄ | |
| | Molecular Weight: 322.75 | |
| WW-178 | 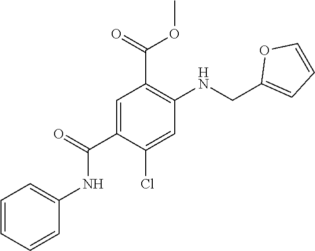 [SMILES: COC(=O)c1cc(C(=O)Nc2ccccc2)c(Cl)cc1NCc1ccco1] | 12% |
| | Chemical Formula: C₂₀H₁₇ClN₂O₄ | |
| | Molecular Weight: 384.82 | |
| WW-179 | 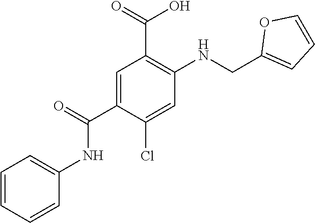 [SMILES: O=C(Nc1ccccc1)c1cc(C(=O)O)c(NCc2ccco2)cc1Cl] | 22% |
| | Chemical Formula: C₁₉H₁₅ClN₂O₄ | |
| | Molecular Weight: 370.79 | |
| WW-187 | 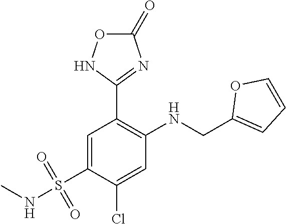 [SMILES: CNS(=O)(=O)c1cc(-c2nc(=O)o[nH]2)c(NCc2ccco2)cc1Cl] | 20% |
| | Chemical Formula: C₁₄H₁₃ClN₄O₅S | |
| | Molecular Weight: 384.79 | |
| 5-LV-54A | 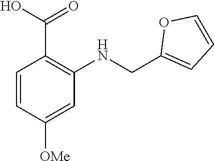 [SMILES: COc1ccc(C(=O)O)c(NCc2ccco2)c1] | 16% |
| | Chemical Formula: C₁₃H₁₃NO₄ | |
| | Molecular Weight: 247.2500 | |
| 5-LV-54B | 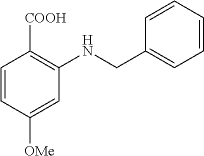 [SMILES: COc1ccc(C(=O)O)c(NCc2ccccc2)c1] | 30% |
| | Chemical Formula: C₁₅H₁₅NO₃ | |
| | Molecular Weight: 257.2890 | |
| LD3-305 | 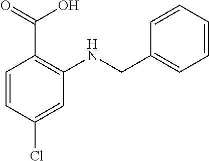 [SMILES: O=C(O)c1ccc(Cl)cc1NCc1ccccc1] | 30% |
| | Chemical Formula: C₁₄H₁₂ClNO₂ | |
| | Molecular Weight: 261.7050 | |
| LD3-306 | 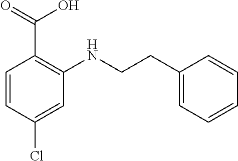 [SMILES: O=C(O)c1ccc(Cl)cc1NCCc1ccccc1] | 21% |
| | Chemical Formula: C₁₅H₁₄ClNO₂ | |
| | Molecular Weight: 275.7320 | |
| LD3-309 | 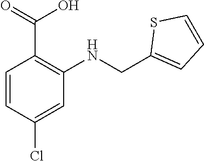 [SMILES: O=C(O)c1ccc(Cl)cc1NCc1cccs1] | 18% |
| | Chemical Formula: C₁₂H₁₀ClNO₂S | |
| | Molecular Weight: 267.7270 | |
| LD3-310 | 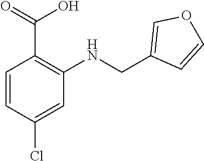 [SMILES: O=C(O)c1ccc(Cl)cc1NCc1ccoc1] | 24% |
| | Chemical Formula: C₁₂H₁₀ClNO₃ | |
| | Molecular Weight: 251.6660 | |
| 5-LV-56A | 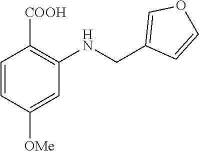 [SMILES: COc1ccc(C(=O)O)c(NCc2ccoc2)c1] | 32% |
| | Chemical Formula: C₁₃H₁₃NO₄ | |
| | Molecular Weight: 247.2500 | |

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A compound, which is a compound of Formula Z: 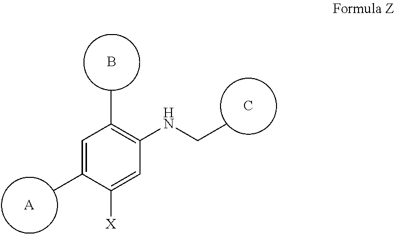
where:
A represents functional region 1;
B represents functional region 2;
C represents functional region 3; and
X represents an alkoxy group, such as methoxy group, or a halide, such as F, Br or Cl,
or a pharmaceutically acceptable salt, solvate or hydrate thereof,
wherein the compound of Formula Z is selected from the group consisting of compounds of Formula I, II, III and IV: 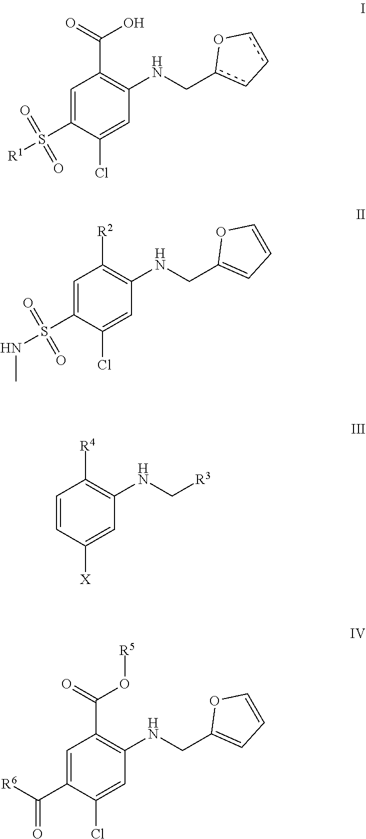
where
R¹ is 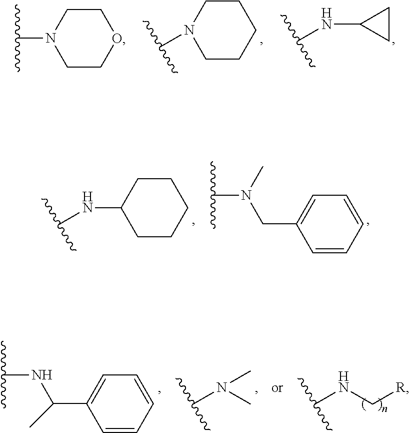
where n is an integer from to 5 and R is CH₃, phenyl or phenyl that is para-substituted with CH₃, OCH₃ or CH(CF₃)OH;
R² is 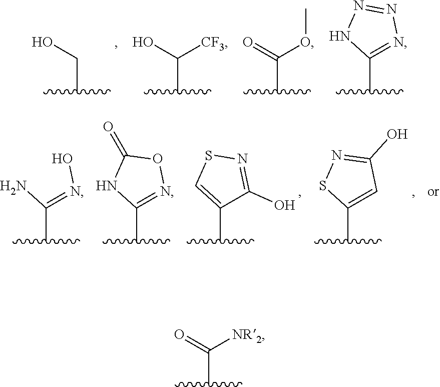
where each R′ is independently H or CH₃;
R³ is 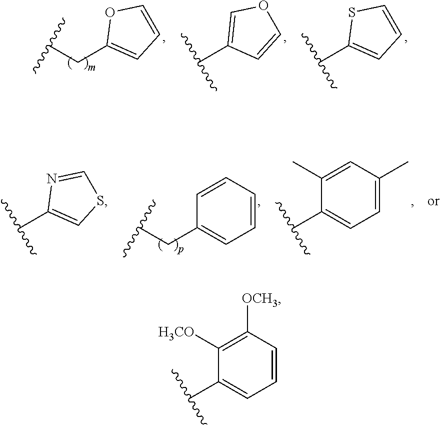
where m is an integer from 0 to 2 and p is an integer of 0 or 1;
R⁴ is —COOH or —CH(CF₃)OH;
R⁵ is H or C₁-C₄ alkyl, preferably H or methyl;
R⁶ is NR⁷R⁸, where each R⁷ and R⁸ is independently selected from H, C₁-C₄ alkyl (preferably methyl or ethyl) or phenyl, or where R⁷ and R⁸ together with the N to which they are bound form a six-membered heterocycle that optionally includes O; and
the dashed lines represent bonds that may be present or absent.

2. The compound of claim 1, or a pharmaceutically acceptable salt thereof, wherein the compound of Formula III is a compound of Formula IIIa:

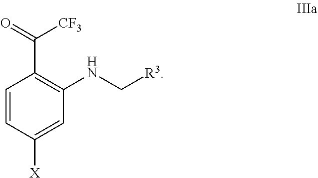

3. The compound of claim 1, or a pharmaceutically acceptable salt thereof, wherein the compound of Formula III is a compound of Formula IIIb:

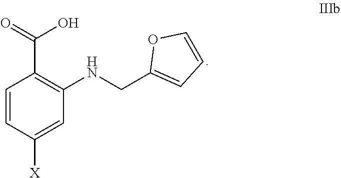

4. The compound of claim 1, or the pharmaceutically acceptable salt thereof, wherein the compound is a compound of formula I, II, or IIIa: 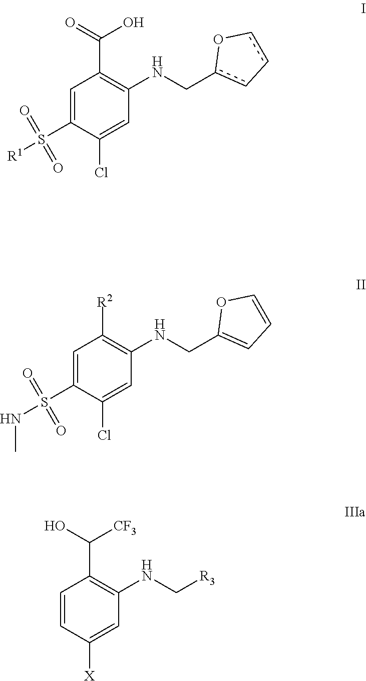
where
R¹ is NHCH₃, 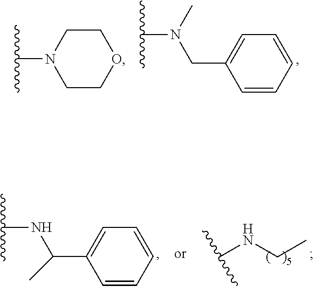
R² is 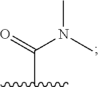 [SMILES: *C(=O)N(C)C]
R³ is 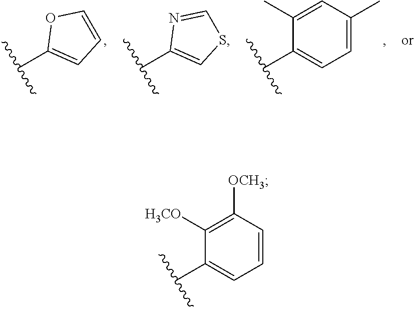
X is F, Cl or Br; and
the dashed lines represent bonds that may be present or absent.

5. The compound of claim 4, wherein the compound is selected from the group consisting of:

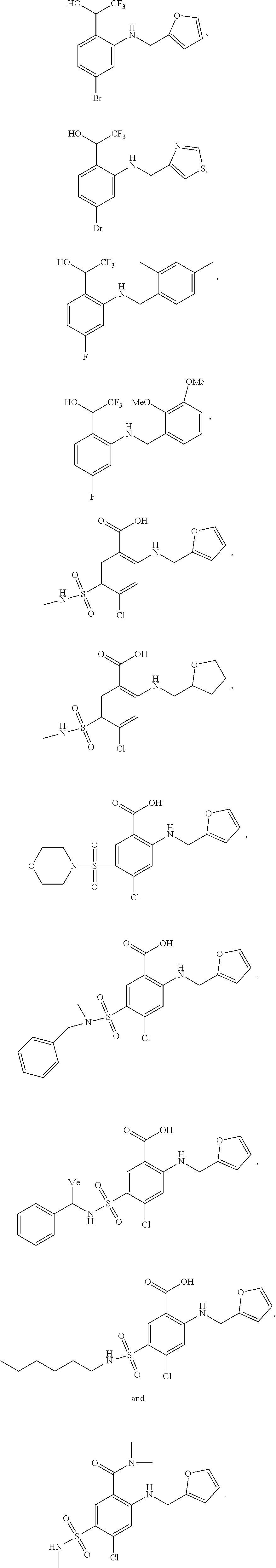

6. The compound of claim 1, wherein the compound of Formula Z is selected from the group consisting of:

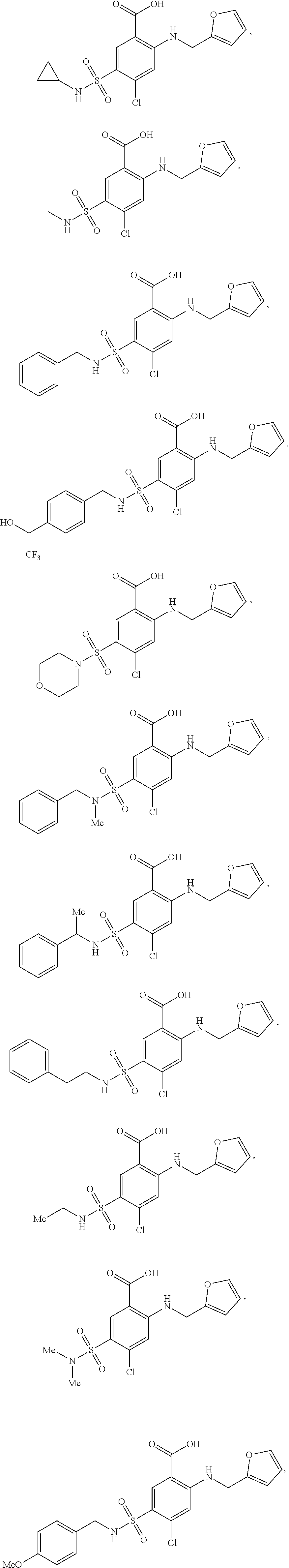

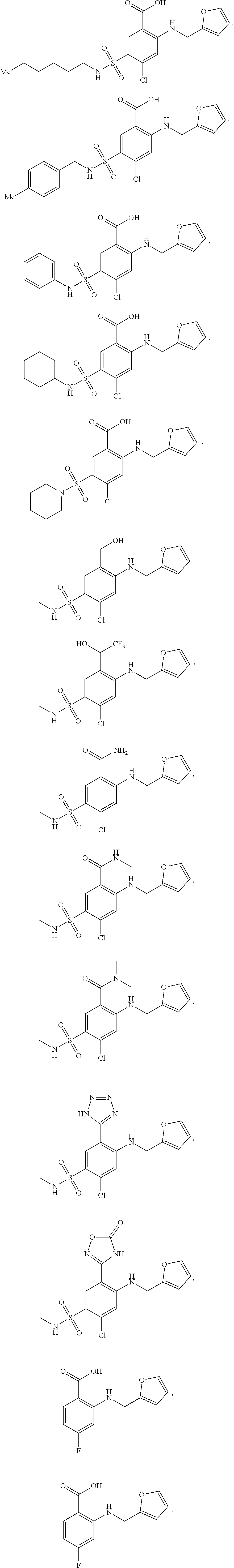

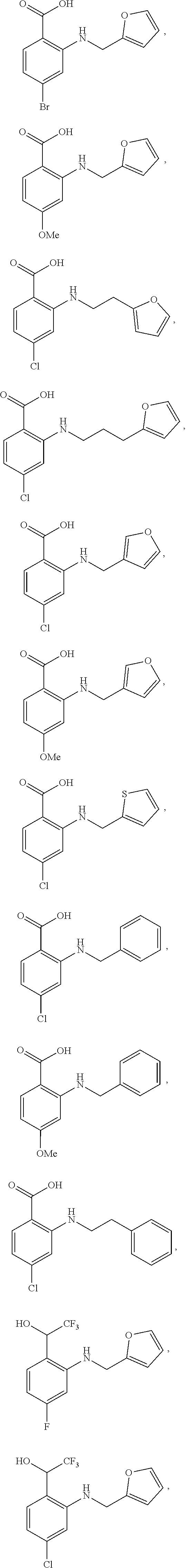

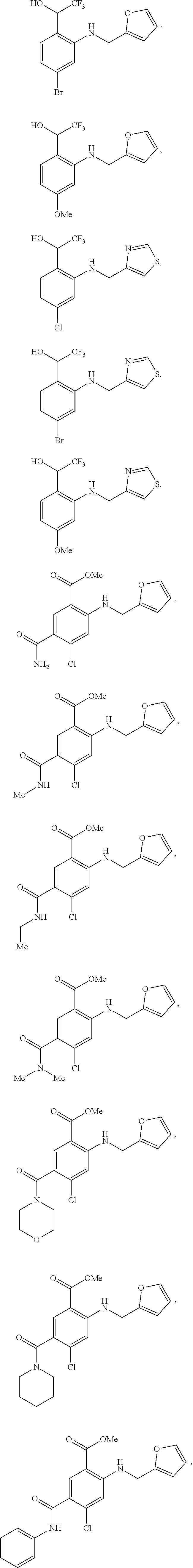

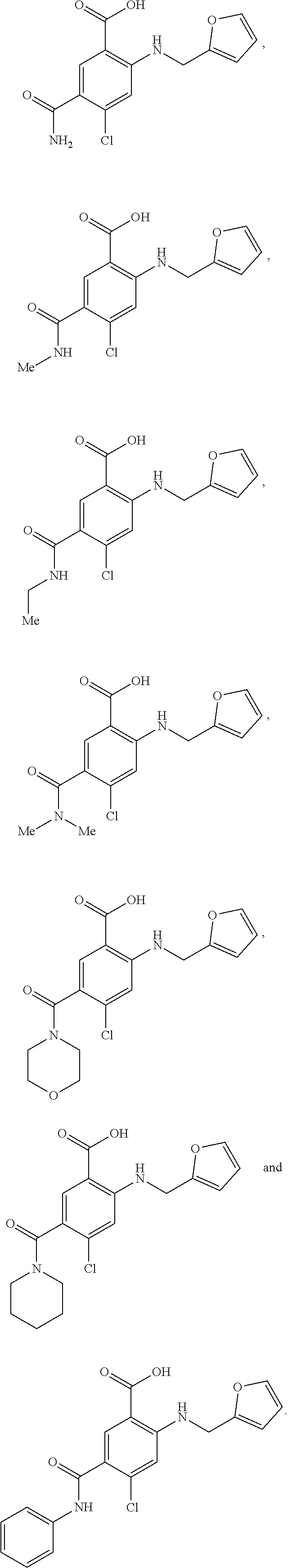

7. The compound of claim 1, for use as an anti-Aβ aggregation agent and/or as an inhibitor of Aβ induced neuroinflammation.

8. (canceled)

9. A method for preventing, delaying or treating Alzheimer's Disease comprising administering furosemide, 2-(benzylamino)benzoic acid, or the compound as defined in claim 1 to a subject in need thereof.

10. A composition comprising one or more compound as defined in claim 1, or a pharmaceutically acceptable salt, solvate or hydrate thereof, and a pharmaceutically acceptable diluent or excipient.

11. The composition of claim 10, wherein the composition further comprises another pharmaceutically active agent for preventing, delaying or treating Alzheimer's Disease.
